(19) United States
(12) Patent Application Publication
(10) Pub. No.: US 2026/0250750 A1
(43) Pub. Date: Aug. 27, 2026

(54) METHOD FOR HIGHLY MULTIPLEXED, THERMAL CONTROLLABLE DNA EXTENSION AND ITS APPLICATIONS

(71) Applicant: PRESIDENT AND FELLOWS OF HARVARD COLLEGE, Cambridge, MA (US)

(72) Inventors: Kuanwei SHENG, Cambridge, MA (US); Xiaokang LUN, Cambridge, MA (US); Hanquan SU, Cambridge, MA (US); Peng YIN, Cambridge, MA (US)

(73) Assignee: PRESIDENT AND FELLOWS OF HARVARD COLLEGE, Cambridge, MA (US)

(21) Appl. No.: 18/995,354

(22) PCT Filed: Jul. 20, 2023
(86) PCT No.: PCT/US2023/028205

§ 371 Date: Jan. 16, 2025

(51) Int. Cl. C12Q 1/6841; C12Q 1/682

(52) U.S. Cl. CPC C12Q 1/6841; C12Q 1/682

(57) ABSTRACT

The methods, compositions and kits described herein provide signal amplification approaches for the detection of target biomolecules that significantly increase the sensitivity of detection using target-binding ligand molecules. The methods include cyclic addition of nucleic acid repeats to, e.g., target-binding molecules in situ, thereby providing multiple landing pads for labeled probes. The methods are well-suited to performance in multiplex, thereby permitting the sensitive detection of multiple targets in a single assay. These methods and compositions can be applied to, among other things, imaging, flow cytometry, CyTOF, and immunoblotting, providing additional tools for research and diagnostic purposes.

### CROSS REFERENCE TO RELATED APPLICATIONS

This application is a 371 National Phase Entry of International Patent Application No. PCT/US23/28205, filed Jul. 20, 2023, which claims benefit under 35 U.S.C. § 119(e) of U.S. Provisional Application No. 63/390,948, filed Jul. 20, 2022, the contents of all of which are incorporated herein by reference in their entireties.

### GOVERNMENT SUPPORT

This invention was made with government support under MH128861 and MH124606 and CA235421 awarded by National Institutes of Health (NIH) and under N00014-18-1-2549 awarded by U.S. Office of Naval Research (NAVY/ONR). The government has certain rights in this invention.

### TECHNICAL FIELD

The technology described herein relates to methods for amplifying signal in detecting, quantifying, and imaging biomolecules.

### BACKGROUND

Methods for detecting, quantitating and/or imaging biological target molecules of interest are central to a wide range of research and diagnostic approaches. There is a need in the art for compositions and methods permitting more sensitive detection of target biomolecules, as well as, for example, detection of a plurality of such target biomolecule in the same assay—so called multiplex detection.

Amplification of nucleic acids allows for the study of many different pathways in biological systems. This amplification can be used in techniques such as imaging, flow cytometry, cytometry by time of flight (CyTOF), immunoblotting, etc. The ability to amplify multiple nucleic acid loci at the same time allows for the examination of different responses and pathways at the same time, applying to both research and diagnostic testing.

### SUMMARY

The technology described herein relates, in general, to the amplification of signal relating to the binding of a target-binding ligand to its target either in situ or ex situ. Such amplification of signal can provide a read-out with sensitivity and resolution to as much as single-molecule detection. In various embodiments, the technology relates to the in situ generation of concatemers of nucleic acid sequence on a target ligand-binding molecule in which the repeated sequences provide hybridization sites for a plurality of labeled nucleic acid probes per target-binding ligand molecule, thereby amplifying the signal related to the binding of target molecules in a cell or tissue sample by the target ligand-binding molecule. The methods, compositions and kits described herein find application in a number of scenarios, but have particular relevance to quantifying the abundance of target molecules and the detection of cellular or sub-cellular locations of target molecules in situ in cell and tissue samples.

In one aspect, provided herein, is a method of labeling a target-binding ligand, the method comprising: (a) providing a conjugate of the target-binding ligand and a first oligonucleotide primer; (b) in a reaction mixture, contacting the conjugated first oligonucleotide primer with a first single-stranded extender template and a nucleic acid polymerase enzyme under conditions permitting hybridization and extension of the first oligonucleotide primer using the first single-stranded extender template; (c) heating the reaction mixture of step (b) to separate the first single-stranded extender template from extended first oligonucleotide primer produced in step (b); (d) repeating steps (b) and (c) at least once, thereby generating a concatemer comprising repeats of oligonucleotide primer sequence, conjugated to the target-binding ligand.

In one embodiment of this and any other aspect described herein, the steps are performed in the order presented. It is noted that while in situ applications of the technology described herein are of particular interest, the signal amplification approach based on concatemers can also be applied in vitro, e. g, in a test tube or other container absent a cell or tissue sample. As non-limiting examples, the concatemer-generating approach can be used to label antibodies or other target-binding ligands (e.g., members of affinity pairs, such as biotin/streptavidin, among others) for use in, e.g., western blotting or other immunoassays. In such embodiments, the concatemers can be generated by extension of an oligonucleotide primer on the target-binding ligand essentially as described herein, except that the target-binding ligand need not be bound to its target. It is also contemplated that concatemers (linear or branched as described further herein below) can be pre-formed, e.g., via thermal cycled extension as described herein, and then associated with a target-binding ligand, e.g., via hybridization and optional cross-linking to an oligonucleotide on the target-binding ligand.

In another embodiment, the first single-stranded extender template comprises a concatemer of at least two head-to-tail copies of a sequence complementary to the first oligonucleotide primer, and a 3′ chain terminator, such that the extender template is not extended by the polymerase.

In another embodiment, the method further comprises, after at least one repeat of steps (b)-(d), the steps of: (i) adding a second oligonucleotide primer and a second single-stranded extender template oligonucleotide, wherein the second oligonucleotide primer comprises, in 5′ to 3′ order, sequence complementary to the first oligonucleotide primer, and sequence complementary to the second single-stranded extender template, wherein the second single-stranded extender template comprises a 3′ chain terminator; (ii) heating the reaction mixture to separate nucleic acid strands and cooling to permit hybridization of the second oligonucleotide primer to concatemer generated in steps (b)-(d) and hybridization of the second single-stranded extender template to the second oligonucleotide primer; (iii) extending the second oligonucleotide primer using the second single-stranded extended template oligonucleotide as template; and (iv) repeating steps (ii) and (iii) at least once, thereby generating a concatemer comprising repeats of the second oligonucleotide primer.

In another embodiment, the method further comprises, after step (d): (i) adding, under conditions permitting hybridization, a nucleic acid comprising: (1) a second concatemer comprised of repeats of a second oligonucleotide sequence; and (2), a sequence complementary to the first oligonucleotide primer, such that a plurality of molecules of the nucleic acid comprising the second concatemer hybridizes via the sequence complementary to the first oligonucleotide primer to the concatemer formed in step (d).

In another embodiment, the method is performed in situ on a cell or tissue sample comprising or being assayed for target ligand.

In another embodiment, the cell or tissue sample is fixed.

In another embodiment, the tissue sample is paraffin embedded.

In another embodiment, the method further comprises contacting a concatemer generated in prior steps with a labeled nucleic acid probe. In one embodiment, the nucleic acid probe comprises sequence complementary to a concatemer repeat. In another embodiment, the nucleic acid probe comprises sequence complementary to the first oligonucleotide primer.

In another embodiment, the method further comprises contacting a concatemer comprised of repeats of the second oligonucleotide primer with a labeled nucleic acid probe, wherein the nucleic acid probe comprises sequence complementary to the second oligonucleotide primer.

In another embodiment, the method further comprises contacting a concatemer comprised of repeats of the second oligonucleotide sequence with a labeled nucleic acid probe, wherein the nucleic acid probe comprises sequence complementary to the second oligonucleotide sequence.

In another embodiment, a plurality of labeled nucleic acid probe molecules hybridizes to a plurality of concatemeric repeats.

In another embodiment, the target-binding ligand comprises an antibody or antigen-binding fragment thereof.

In another embodiment, a plurality of different target-binding ligands are labeled in multiplex via different orthogonal oligonucleotide primer/single-stranded extender template pairs.

In another aspect, provided herein is a method of detecting a target molecule in situ in a preparation of cells or tissue, the method comprising: (a) contacting the preparation of cells or tissue with a target-binding ligand conjugated to a first oligonucleotide primer, under conditions permitting specific binding of the target-binding ligand to the target molecule; (b) adding to the preparation of cells or tissue a reaction mixture comprising a first single-stranded extender template and a nucleic acid polymerase enzyme under conditions permitting hybridization and extension of the first oligonucleotide primer using the first single-stranded extender template; (c) heating the reaction mixture of step (b) to separate the first single-stranded extender template from extended first oligonucleotide primer produced in step (b); (d) repeating steps (b) and (c) at least once, thereby generating a concatemer comprising repeats of the first oligonucleotide primer sequence, conjugated to the target-binding ligand; (e) contacting the concatemer generated in step (d) with a labeled nucleic acid probe; and (f) detecting labeled nucleic acid probe, wherein detection of labeled nucleic acid probe indicates the presence and location of the target molecule in the preparation of cells or tissue.

In one embodiment of this aspect, the first single-stranded extender template comprises a concatemer of at least two head-to-tail copies of a sequence complementary to the first oligonucleotide primer, and a 3′ chain terminator, such that the extender template is not extended by the polymerase.

In another embodiment, the steps are performed in the order presented.

In another embodiment, the target-binding ligand comprises an antibody or antigen-binding fragment thereof.

In another embodiment, the labeled nucleic acid probe comprises sequence complementary to the first oligonucleotide primer.

In another embodiment, the labeled nucleic acid probe comprises sequence complementary to a concatemer repeat.

In another embodiment, a plurality of labeled nucleic acid probe molecules hybridizes to a plurality of concatemer repeats conjugated to the target-binding ligand.

In another embodiment, the method further comprises the steps, after step (d), and before step (e), of: (i) adding a second oligonucleotide primer and a second single-stranded extender template oligonucleotide, wherein the second oligonucleotide primer comprises, in 5′ to 3′ order, sequence complementary to the first oligonucleotide primer, and sequence complementary to the second single-stranded extender template, wherein the second single-stranded extender template comprises a 3′ chain terminator; (ii) heating the reaction mixture to separate nucleic acid strands and cooling to permit hybridization of the second oligonucleotide primer to concatemer generated in step (d) and hybridization of the second single-stranded extender template to the second oligonucleotide primer; (iii) extending the second oligonucleotide primer using the second single-stranded extended template oligonucleotide as template; and (iv) repeating steps (ii) and (iii) at least once, thereby generating a concatemer comprising repeats of the second oligonucleotide primer.

In another embodiment, the labeled nucleic acid probe comprises sequence complementary to the second oligonucleotide primer.

In another embodiment, a plurality of labeled nucleic acid probe molecules hybridizes to a plurality of concatemer repeats conjugated to the target-binding ligand.

In another embodiment, the method further comprises, after step (d) and before step (e): (i) adding, under conditions permitting hybridization, a nucleic acid comprising: (1) a second concatemer comprised of repeats of a second oligonucleotide sequence; and (2), a sequence complementary to the first oligonucleotide primer, such that a plurality of molecules of the nucleic acid comprising the second concatemer hybridizes via the sequence complementary to the first oligonucleotide primer to the concatemer comprising repeats of the first oligonucleotide primer sequence formed in step (d).

In another embodiment, the labeled nucleic acid probe comprises sequence complementary to the second oligonucleotide sequence.

In another embodiment, a plurality of labeled nucleic acid probe molecules hybridizes to a plurality of second concatemer repeats.

In another embodiment, the preparation of cells or tissue is fixed.

In another embodiment, the preparation of tissue is paraffin embedded.

In another aspect, provided herein is a method of labeling a set of target-binding ligands, the method comprising: (a) providing a set of orthogonal first oligonucleotide primer and first single-stranded extender template oligonucleotide pairs, wherein the first oligonucleotide primer of each pair has a different sequence from other first oligonucleotide primers in the set, and is conjugated to a different member of a set of target-binding ligands; (b) in a reaction mixture, contacting the conjugated first oligonucleotide primers with the first single-stranded extender templates of the set of orthogonal pairs and a nucleic acid polymerase under conditions permitting hybridization and extension of the first oligonucleotide primers using the first single-stranded extender templates of the set of orthogonal pairs; (c) heating the reaction mixture of step (b) to separate the first single-stranded extender templates from extended first oligonucleotide primers produced in step (b); (d) repeating steps (b) and (c) at least once, thereby generating, on each member of the set of target ligands, a concatemer comprising repeats of oligonucleotide primer sequence, conjugated to the target-binding ligand.

In one embodiment, the first single-stranded extender template in each orthogonal first oligonucleotide primer/first single-stranded extender pair comprises a concatemer of at least two head-to-tail copies of a sequence complementary to the first oligonucleotide primer, and a 3′ chain terminator, such that the extender template is not extended by the polymerase.

In another embodiment, the steps are performed in the order presented.

In another embodiment, the method further comprises, after step (d), the steps of: (i) adding a second set of orthogonal second oligonucleotide primer, second single-stranded extender template oligonucleotide pairs, wherein the second oligonucleotide primer in each pair comprises, in 5′ to 3′ order, sequence complementary to a member of the set of first oligonucleotide primers, and sequence complementary to the second single-stranded extender template of the second set of orthogonal oligonucleotide pairs, wherein each second single-stranded extender template comprises a 3′ chain terminator; (ii) heating the reaction mixture to separate nucleic acid strands and cooling to permit hybridization of the second oligonucleotide primers to concatemers generated in steps (b)-(d) and hybridization of the second single-stranded extender templates to the second oligonucleotide primers; (iii) extending the second oligonucleotide primers using the second single-stranded extended template oligonucleotides as template; and (iv) repeating steps (ii) and (iii) at least once, thereby generating, on respective members of the set of target-binding ligands, a concatemer comprising repeats of a respective second oligonucleotide primer.

In another embodiment, the method further comprises contacting concatemers generated in prior steps with a plurality of labeled nucleic acid probes, wherein each different probe is labeled with a distinguishable detectable label moiety, and wherein members of the plurality of labeled nucleic acid probes comprise sequence complementary to respective members of the set of first oligonucleotide primers.

In another embodiment, the method further comprises contacting concatemers generated in step (iv) with a plurality of labeled nucleic acid probes, wherein each different probe is labeled with a distinguishable detectable label moiety, and wherein members of the plurality of labeled nucleic acid probes comprise sequence complementary to respective members of the set of second oligonucleotide primers.

In another embodiment, for respective members of the set of target-binding ligands, a plurality of labeled nucleic acid probe molecules hybridizes to a plurality of concatemeric repeats.

In another embodiment, the method is performed in situ on a cell or tissue sample comprising or being assayed for target ligand.

In another embodiment, the preparation of cells or tissue is fixed.

In another embodiment, the preparation of tissue is paraffin embedded.

In another embodiment, the target-binding ligand comprises an antibody or antigen-binding fragment thereof.

In another aspect, provided herein is a method of detecting a set of target molecules in situ in a preparation of cells or tissue, the method comprising: (a) providing a set of target-binding ligand molecules, wherein members of the set of target-binding ligand molecules are conjugated to respective first oligonucleotide primer members of a set of orthogonal first oligonucleotide primer and first single-stranded extender template oligonucleotide pairs, wherein the first oligonucleotide primer of each pair has a different sequence from other first oligonucleotide primers in the set, and is conjugated to a different member of the set of target-binding ligand molecules; (b) contacting the preparation of cells or tissue with the set of target-binding ligand molecules under conditions permitting specific binding of the target-binding ligand molecules to target molecules present in the preparation of cells or tissue; (c) adding to the preparation of cells or tissue a reaction mixture comprising first single-stranded extender templates of the set of orthogonal first oligonucleotide primer and first single-stranded extender template oligonucleotide pairs, and a nucleic acid polymerase, under conditions permitting hybridization and extension of the first oligonucleotide primers using the respective first single-stranded extender templates; (d) heating the reaction mixture of step (c) to separate the first single-stranded extender templates from extended first oligonucleotide primers produced in step (c); (e) repeating steps (c) and (d) at least once, thereby generating, on respective members of the set of target-binding ligand molecules, a concatemer comprising repeats of the respective first oligonucleotide primer sequence, conjugated to the target-binding ligand molecule; (f) contacting the concatemers generated in step (e) with a plurality of distinguishably-labeled nucleic acid probes; and (g) detecting labeled nucleic acid probes, wherein detection of labeled nucleic acid probes indicates the presence and location of the target molecules in the preparation of cells or tissue.

In one embodiment, the steps are performed in the order presented.

In another embodiment, the first single-stranded extender template comprises a concatemer of at least two head-to-tail copies of a sequence complementary to the first oligonucleotide primer, and a 3′ chain terminator, such that the extender template is not extended by the polymerase.

In another embodiment, the method further comprises, after step (e), and before step (f), the steps of: (i) adding a second set of orthogonal second oligonucleotide primer, second single-stranded extender template oligonucleotide pairs, wherein the second oligonucleotide primer in each pair comprises, in 5′ to 3′ order, sequence complementary to a member of the set of first oligonucleotide primers, and sequence complementary to the second single-stranded extender template of the second set of orthogonal oligonucleotide pairs, wherein each second single-stranded extender template comprises a 3′ chain terminator; (ii) heating the reaction mixture to separate nucleic acid strands and cooling to permit hybridization of the second oligonucleotide primers to concatemers generated in steps (c)-(e) and hybridization of the second single-stranded extender templates to the second oligonucleotide primers; (iii) extending the second oligonucleotide primers using the second single-stranded extended template oligonucleotides as template; and (iv) repeating steps (ii) and (iii) at least once, thereby generating, on respective members of the set of target-binding ligands, a concatemer comprising repeats of a respective second oligonucleotide primer.

In another embodiment, the plurality of distinguishably-labeled nucleic acid probes comprises sequences complementary to the respective first oligonucleotide primer members in the set of orthogonal first oligonucleotide primer and first single-stranded extender template oligonucleotide pairs.

In another embodiment, the plurality of distinguishably-labeled nucleic acid probes comprises sequences complementary to respective second oligonucleotide primer members of the second set of orthogonal second oligonucleotide primer, second single-stranded extender template oligonucleotide pairs.

In another embodiment, the target-binding ligand comprises an antibody or antigen-binding fragment thereof.

In another embodiment, a plurality of labeled nucleic acid probe molecules hybridizes to a plurality of concatemer repeats conjugated to the target-binding ligand.

In another aspect, provided herein is a kit comprising reagents for performing one or more of the methods described herein.

In another aspect, provided herein, is a target-binding ligand comprising a concatemer produced by a method as described herein. In one embodiment, the target-binding ligand comprises an antibody or antigen-binding fragment thereof.

In another aspect, provided herein is a method of generating a nucleic acid strand comprising concatemeric repeats of a given sequence, the method comprising: a) providing in a reaction mixture, a first oligonucleotide primer and a first extender template oligonucleotide comprising a concatemer of at least two head-to-tail copies of sequence complementary to the first oligonucleotide primer, wherein the first extender template comprises a chain terminator at its 3′ end; b) incubating the reaction mixture under conditions permitting hybridization of the first oligonucleotide primer to the first extender template oligonucleotide; c) extending the hybridized first primer with a nucleic acid polymerase enzyme, thereby generating an extended nucleic acid strand complementary to the first extender template; d) heating the reaction mixture to separate the extended nucleic acid strand complementary to the first extender template from the first extender template; e) cooling the heated products to permit hybridization of the first extender template oligonucleotide to the extended nucleic acid strand complementary to the first extender template generated in step (c); f) extending the extended nucleic acid strand complementary to the first extender template generated in step (c) with the polymerase, thereby generating an extended nucleic acid strand comprising concatemeric repeats of the sequence comprised by the first oligonucleotide primer.

In one embodiment, the method further comprises repeating steps (d) to (f) at least once.

In another embodiment, the steps are performed in the order presented.

In another embodiment, the method comprises repeating steps (d) to (f) at least n times, wherein each iteration of steps (d) to (f) increases the concatemeric nucleic acid length by one concatemeric repeat.

In another embodiment, step (a) comprises providing a set of orthogonal first oligonucleotide primer and first extender template oligonucleotide pairs, such that steps (b)-(f) generate a multiplex set of concatemers, each comprising concatemeric repeats of the respective orthogonal oligonucleotide primer sequences.

In another embodiment, the method further comprises, after one or more repetitions of steps (d) to (f): i) adding a second oligonucleotide primer and a second extender template oligonucleotide to the reaction, wherein the second oligonucleotide primer comprises a 5′ proximal sequence complementary to the first oligonucleotide primer sequence and a 3′ proximal sequence complementary to one of at least two repeat portions of the second extender template oligonucleotide, wherein the second extender template oligonucleotide comprises at least two head-to-tail repeats of sequence complementary to the second oligonucleotide primer and a chain terminator at its 3′ end; ii) hybridizing the second oligonucleotide primer to one or more concatemeric repeats on a concatemer formed after one or more repetitions of steps (d) to (f); iii) hybridizing the second extender template oligonucleotide to the second oligonucleotide primer; iv) extending the second oligonucleotide primer with the polymerase enzyme, thereby generating an extended nucleic acid strand complementary to the second extender template; v) heating the reaction mixture to separate the extended nucleic acid strand complementary to the second extender template from the second extender template; vi) cooling the heated products to permit hybridization of the second extender template oligonucleotide to the extended nucleic acid strand complementary to the second extender template generated in step (iv); vii) extending the extended nucleic acid strand complementary to the second extender template generated in step (iv) with the polymerase, thereby generating an extended nucleic acid strand comprising concatemeric repeats of the sequence comprised by the second oligonucleotide primer.

In another embodiment, the method further comprises repeating steps (v) to (vii) at least once.

In another embodiment, step (i) comprises providing a set of orthogonal second oligonucleotide primer and second extender template oligonucleotide pairs, such that steps (ii)-(vii) generate a multiplex set of concatemers, each comprising concatemeric repeats of the respective orthogonal oligonucleotide primer sequences.

In another embodiment, the first oligonucleotide primer is conjugated to a target-binding ligand via its 5′ end.

In another embodiment, the target-binding ligand is a polypeptide.

In another embodiment, the polypeptide comprises an antibody or antigen-binding fragment thereof.

In another embodiment, the method further comprises contacting the target-binding ligand with a cell or tissue preparation comprising or being assayed for the presence of the target.

In another embodiment, the contacting is performed before concatemer-generating steps (a)-(f) or (i)-(vii).

In another embodiment, the method further comprises contacting an extended nucleic acid strand comprising concatemeric repeats generated according to steps (a)-(f) and/or (i)-(vii) with a nucleic acid probe comprising the complement of a concatemeric repeat sequence, and a detectable label.

In another embodiment, the method generates linear concatemers comprising the first oligonucleotide primer sequence.

In another embodiment, the method generates branched concatemers comprising concatemers of the first oligonucleotide primer sequence, complexed with concatemers of the second oligonucleotide primer sequence.

In another embodiment, the method is performed in contact with a cell or tissue preparation.

In another embodiment, the cell or tissue preparation is fixed.

In another aspect, provided herein is a method of labeling a target-binding ligand, the method comprising: performing the method of any one of the preceding embodiments, wherein the first oligonucleotide primer is conjugated to the target-binding ligand.

In one embodiment, the target-binding ligand comprises an antibody or antigen-binding fragment thereof.

In another embodiment, the first oligonucleotide primer is conjugated to the target binding ligand in a manner that permits extension of the primer from its 3′ end.

In another embodiment, the method is performed in contact with a cell or tissue preparation.

In another embodiment, the cell or tissue preparation is fixed.

In another embodiment, the method further comprises contacting the concatemer generated in any of the preceding claims with a labeled nucleic acid probe, and detecting labeled nucleic acid probe associated with concatemer, wherein detection of labeled nucleic acid probe indicates the presence and location of the target molecule.

In another embodiment, the nucleic acid polymerase enzyme is a thermostable nucleic acid polymerase enzyme.

In one aspect, described herein is a method of generating a nucleic acid molecule, the method comprising: (a) providing a first oligonucleotide primer; (b) in a reaction mixture, contacting the first oligonucleotide primer with a first single-stranded extender template and a nucleic acid polymerase enzyme under conditions permitting hybridization and extension of the first oligonucleotide primer using the first single-stranded extender template, wherein the first single-stranded extender template comprises, in a 5′ to 3′ direction, a first extension template element, a sequence complementary to the first oligonucleotide primer and a 3′ chain terminator, such that the extender template is not extended by the polymerase; (c) heating the reaction mixture of step (b) to separate the first single-stranded extender template from extended first oligonucleotide primer produced in step (b); (d) contacting the extended first oligonucleotide primer with a second single-stranded extender template under conditions permitting hybridization and extension of the first extended oligonucleotide primer generated in step (b) using the second single-stranded extender template, wherein the second single-stranded extender template comprises, in a 5′ to 3′ direction, a second extension template element, a copy of the first extension template element, and a 3′ chain terminator; wherein steps (a)-(d) generate a nucleic acid molecule comprising, in 5′ to 3′ order, the first oligonucleotide primer sequence, the complement of the first extension template element, and the complement of the second extension template element.

In one embodiment, the method further comprises repeating steps (d) to (f) at least once.

In another embodiment, the steps are performed in the order presented.

In another embodiment, the method comprises repeating steps (d) to (f) at least n times, wherein each iteration of steps (d) to (f) increases the concatemeric nucleic acid length by one concatemeric repeat.

In another embodiment, step (a) comprises providing a set of orthogonal first oligonucleotide primer and first extender template oligonucleotide pairs, such that steps (b)-(f) generate a multiplex set of concatemers, each comprising concatemeric repeats of the respective orthogonal oligonucleotide primer sequences.

In another embodiment, the method further comprises, after one or more repetitions of steps (d) to (f): i) adding a second oligonucleotide primer and a second extender template oligonucleotide to the reaction, wherein the second oligonucleotide primer comprises a 5′ proximal sequence complementary to the first oligonucleotide primer sequence and a 3′ proximal sequence complementary to one of at least two repeat portions of the second extender template oligonucleotide, wherein the second extender template oligonucleotide comprises at least two head-to-tail repeats of sequence complementary to the second oligonucleotide primer and a chain terminator at its 3′ end; ii) hybridizing the second oligonucleotide primer to one or more concatemeric repeats on a concatemer formed after one or more repetitions of steps (d) to (f); iii) hybridizing the second extender template oligonucleotide to the second oligonucleotide primer; iv) extending the second oligonucleotide primer with the polymerase enzyme, thereby generating an extended nucleic acid strand complementary to the second extender template; v) heating the reaction mixture to separate the extended nucleic acid strand complementary to the second extender template from the second extender template; vi) cooling the heated products to permit hybridization of the second extender template oligonucleotide to the extended nucleic acid strand complementary to the second extender template generated in step (iv); vii) extending the extended nucleic acid strand complementary to the second extender template generated in step (iv) with the polymerase, thereby generating an extended nucleic acid strand comprising concatemeric repeats of the sequence comprised by the second oligonucleotide primer.

In another embodiment, the polymerase enzyme is a thermostable polymerase enzyme.

In another embodiment, the method further includes repeating steps (b) and (c) at least once before step (d), wherein each repeat of steps (b) and (c) extends the generated nucleic acid molecule by one copy of the complement of the first extension template element.

In another embodiment, the method further includes, at least one iteration of (e) heating the reaction mixture of step (d) to separate the second single-stranded extender template from extended first oligonucleotide primer produced in step (d); and (f) repeating step (d), wherein each iteration of steps (e) and (f) extends the generated nucleic acid molecule by one copy of the complement of the second extension element.

In another embodiment, the method further comprises heating the reaction mixture after step (d) and contacting the nucleic acid molecule generated in steps (a) to (d) with at least one additional single-stranded extender template under conditions permitting hybridization and extension of the generated nucleic acid molecule, wherein the additional single-stranded extender template comprises, in a 5′ to 3′ direction, an additional extension template element, a copy of the extension template element from the previous round of extension, and a 3′ chain terminator, wherein the generated nucleic acid molecule is extended to include the complement of the additional extension template element.

In another embodiment, the first oligonucleotide is conjugated to a target-binding ligand.

In another embodiment, the method is performed in contact with a cell or tissue sample.

In another embodiment, the method further comprises contacting the generated nucleic acid molecule with a labeled nucleic acid probe comprising sequence of one or more of the extension template elements.

In another aspect, provided herein, is a kit for performing one or more of the methods as described herein, the kit comprising: a) a first oligonucleotide primer or a set of first oligonucleotide primers; and b) a single-stranded extender template or a set of single-stranded extender templates or an ordered extension template nucleic acid molecule.

In one embodiment, the first oligonucleotide primer or the set of first oligonucleotide primers and single stranded extender template or the set of single stranded extender templates are orthogonal sets.

In another embodiment, the orthogonal sets are optimized to avoid primer/extender cross-talk, primer-dimer formation, and off-target hybridization.

In another embodiment, the single-stranded extender template or the set of single-stranded extender templates are suitable for performing a concatemer-generating method.

In another embodiment, the kit further comprises packaging materials for the various components, and optionally, instructions for use.

In another embodiment, the kit further comprises one or more target-binding ligand molecules or reagents for conjugating a first oligonucleotide to a target-binding ligand.

In another embodiment, the kit further comprises a thermostable polymerase, nucleotides, reaction buffer components, and reagents for labeling a nucleic acid probe molecule.

In another embodiment, the kit further comprises a probe molecule complementary to a concatemeric repeat element.

In another embodiment, the probe molecule can be complementary to one or more elements in an ordered extension product generated.

### DEFINITIONS

As used herein, the term “target-binding ligand” refers to a molecule or moiety that specifically binds a given target molecule. Target-binding ligands can include, for example, peptides, polypeptides, nucleic acids, aptamers, a receptor and/or its cognate ligand, members of an affinity binding pair (including, but not limited to biotin/streptavidin), and small molecule agents that specifically bind a target molecule as that term is defined herein. Antibodies and antigen-binding fragments or constructs thereof represent one class of target-binding ligands useful in the methods, compositions and kits described herein. Nucleic acids comprising sequence complementary to a given target DNA or RNA molecule (including, but not limited to an mRNA molecule) represent another class of target-binding ligands that can be useful in the methods, compositions and kits described herein.

As used herein, the term “specific binding” refers to a physical interaction between two molecules, compounds, cells and/or particles wherein the first entity binds to the second, target entity with greater specificity and affinity than it binds to a third entity which is a non-target. A reagent specific for a given target is one that exhibits specific binding for that target under the conditions of the assay being utilized. The specificity of an antibody or antibody fragment thereof can be determined based on affinity and/or avidity. The affinity, represented by the equilibrium constant for the dissociation (KD) of an antigen with an antigen-binding protein, is a measure of the binding strength between an antigenic determinant and an antigen-binding site on the antigen-binding protein, such as an antibody or antigen-binding fragment thereof: the less the value of the KD, the stronger the binding strength between an antigenic determinant and the antigen-binding molecule. Alternatively, the affinity can also be expressed as the affinity constant (KA), which is 1/KD). Accordingly, an antibody or antigen-binding fragment thereof as described herein is said to be “specific for” or to “specifically bind” or “selectively bind” a first target or antigen compared to a second target or antigen when it binds to the first antigen with an affinity (as described above, and suitably expressed, for example as a KD value) that is at least 1000 times, 10000 times or more better than the affinity with which said amino acid sequence or polypeptide binds to another given polypeptide. Generally, a molecule that “specifically binds,” “selectively binds” or “is specific for” a given target will bind with a KD of 10-5 M (10000 nM) or less, e.g., 10-6 M, 10 7 M, 10 8 M, 10 9 M, 10-10 M, 10-11 M, 10-12 M, or less. Specific binding can be influenced by, for example, the affinity and avidity of the polypeptide agent and the concentration of polypeptide agent. The person of ordinary skill in the art can determine appropriate conditions under which polypeptide agents as described herein selectively bind the target using any suitable methods, such as titration of a polypeptide agent in a suitable cell binding assay.

It should be understood in this context that where antibodies or antigen-binding fragments thereof are concerned, the specific binding is mediated by the CDRs of the antibody polypeptide, as opposed to any other portion of the antibody polypeptide. Antibody dissociation constants and affinities can be determined, for example, by a surface plasmon resonance based assay (such as the BIAcore assay described in PCT Application Publication No. WO2005/012359); Forte Bio Octet™ analysis, enzyme-linked immunosorbent assay (ELISA); and competition assays (e.g., RIA's), for example.

As used herein, the term “conjugated” refers to the linkage of, for example, an oligonucleotide to a target-binding ligand in a manner that is stable through steps of thermal cycling to generate concatemers or ordered nucleic acid extension products as described herein. Conjugates can include covalent linkages. In various embodiments, conjugates can include a linker molecule between the target-binding ligand and the conjugated oligonucleotide.

As used herein the term “concatemer” refers to a nucleic acid molecule comprising two or more repeats of a given sequence in a head-to-tail, 5′ to 3′ orientation. Concatemers can include two or more, three or more, four or more, five or more, six or more, seven or more, eight or more, nine or more, ten or more, 15 or more, 20 or more, 25 or more, 30 or more, 35 or more, 40 or more, 45 or more, or 50 or more repeats of the given sequence. The number of repeats for concatemers generated as described herein is determined by the number of cycles of strand separation and primer extension employed. Repeat unit lengths of concatemers as described herein can determine the degree of multiplexing achievable for orthogonal sets of repeats of a given length. The repeat units in a concatemer as described herein can be, for example, 8 or more, 9 or more, 10 or more, 11 or more, 12 or more, 13 or more, 14 or more, 15 or more, 16 or more, 17 or more, 18 or more, 19 or more, 20 or more, 25 or more, 30 or more, 35 or more, 40 or more, 45 or more, or 50 or more nucleotides in length.

As used herein, the term “conditions permitting hybridization and extension” of a given molecule, e.g., an oligonucleotide molecule, refers to conditions of temperature, salt, buffer and other reaction components sufficient for a template-directed polymerase-mediated primer extension reaction. Exact conditions for extension by a given polymerase vary with the enzyme chosen, but are known in the art and/or can be determined without undue experimentation. Conditions for hybridization, e.g., of a primer as described herein to a single-stranded extender template as described herein, will generally be those salt, buffer and other reaction component conditions appropriate for the chosen polymerase enzyme, with the annealing or hybridization temperature established by one of skill in the art primarily on the basis of the length of the concatemer repeat unit and degree of multiplexing in a given reaction. Thus, where performed in uniplex, with one given molecule being labeled with a single concatemeric sequence in the reaction, an annealing or hybridization temperature can be determined based on the Tm of the specific concatemer repeat sequence—generally the annealing temperature in a cycling reaction is about 5 oC below the Tm for the hybridization of the repeat sequence to its complement under the salt and reaction component concentrations optimal for the enzyme of choice. Where performed in multiplex, the length of the repeat unit takes on added importance, as the Tm for various repeat sequences will vary. Primer/single-stranded extended template design can take sequence variation into account to design primers and single-stranded extender templates that have Tm values that are relatively close to each other, generally on the order of within 5-7 oC for all members of a set of primer/extender template sequences. Under these circumstances, a single annealing or hybridization temperature in a cycling reaction can permit efficient hybridization and extension of members of the set in multiplex. In various embodiments of a method including a step of contacting a nucleic acid molecule with a single-stranded extender template under conditions permitting hybridization and extension of the nucleic acid molecule, the reaction can be incubated at an annealing temperature for a period of time (generally a matter of seconds to minutes) before raising the temperature to an optimum extension temperature for the polymerase enzyme. It should be understood that contacting a nucleic acid molecule with a single-stranded extender template “under conditions permitting hybridization and extension” of the nucleic acid molecule can include such an annealing/extending temperature shift. In some embodiments, annealing or hybridization occurs efficiently at a temperature at which the polymerase enzyme will be sufficiently active as not to require such a temperature shift; whether or not such a shift is needed in a given circumstance will be apparent to the one of ordinary skill in the art depending upon the enzyme used, and can also be determined empirically without undue experimentation.

### BRIEF DESCRIPTION OF THE DRAWINGS

FIG. 1A-1C. The schemes for thermal controllable DNA extension. FIG. 1A shows thermal cycling extension (TCE) can be used to generate concatemers with repeated sequences. FIG. 1B shows TCE to achieve multiplexed concatemer generation. FIG. 1C shows TCE can also be used for primer exchange reactions.

FIG. 2 depicts the number of maximum multiplexity for different primer length including 9 nt: 93; 10 nt: 170; 11 nt: 315; 12 nt: 585; 13 nt: 1092; 14 nt: 2048; and 15 nt: 3855.

FIG. 3 shows a highly controllable amplification. Two randomly selected sequences were used for TCE reactions. 30, 60, 90, 120 cycles of amplification were performed. The size of the concatemer correspond with cycle number of amplification.

FIG. 4 exhibits high amplification uniformity. 50 different 9 bp primers were tested for their amplification uniformity in test tubes. 5 nM of each primer was added to different tubes with 1 μM of corresponding single-stranded extender (“ssEXTer”). Each tube was amplified for 80 cycles. The two dashed lines are 350 bp and 500 bp respectively.

FIG. 5 shows highly multiplexed amplification. Here 50 primers were added and their ssEXTers in a tube. The sample was amplified for 120 cycles and sent it for next generation sequencing. The frequency was counted of all 50 sequences for each primer after each extension repeat. The results showed 45 ssEXTers have >99.5% specificity and 48 ssEXTers have >99% specificity.

FIG. 6 depicts a schematic showing the principle of multiplexing imaging via in-situ extension.

FIG. 7A-7B. FIG. 7A shows in situ extension (ISE) signal amplification on protein targets. FIG. 7B shows ISE signal amplification on RNA targets. The signal amplification is tuned by changing thermo-cycle numbers.

FIG. 8 demonstrates that branching ISE imaging makes >100-fold signal amplification. Contrast is adjusted to avoid over-saturating the images.

FIG. 9 identifies protein expression level via multiplexing ISE imaging on Py2T model cell.

FIG. 10A-10B. FIG. 10A shows the co-imaging of protein and RNA in Hela cell using ISE imaging. FIG. 10B shows the co-imaging of protein and RNA in mouse brain fresh frozen section using ISE imaging.

FIG. 11 examines an ISE protocol that enables labeling of neuronal marker in thick (0.5 mm) mouse brain tissue.

FIG. 12A-12B. FIG. 12A examines an ISE that shows high signal-to-noise with low non-specific fluorescence background and enables single molecule imaging, where the majority of the signal puncta in the cells are observed to colocalize with high Tubulin-GFP expression region. FIG. 12B examines an ISE that shows high signal-to-noise with low non-specific fluorescence background and enables single molecule imaging, where signal amplification by fluorophore-tagged secondary antibody leads to high non-specific background signal in the region lacking Tubulin-GFP expression.

FIG. 13A-13B. FIG. 13A is a schematic of epitope detection with conventional mass cytometry antibodies. FIG. 13B is a schematic of epitope detection with amplification by cyclic extension of DNA oligo (ACED) amplification method.

FIG. 14 depicts CNvK-based photocrosslinking which allows hybridized detection strands to stay intact during mass cytometry sample acquisition.

FIG. 15A-15C. FIG. 15A shows transient transfection was used to generate an expression gradient of GFP in a population of cells that were subsequently targeted by an anti-GFP antibody conjugated with ACED oligos. Signal amplification by ACED (y-axis) through 1-500 thermal cycles was compared with a counterstaining using anti-rat secondary antibodies to show the specificity of ACED. FIG. 15B examines data in FIG. 15A which was divided into 10 equal-width bins according to their GFP expression level showed by the secondary antibody. Bin 1 indicates the untransfected cells as internal control. Bin 10 shows the cells with the highest GFP expression level. FIG. 15C examines bin medians and signal-to-noise ratio (i.e., medians of bin 1-9 compared to medians in bin 1) across the 1-500 thermal cycles are demonstrated in the left two plots, indicating a 13-fold amplification strength and a six-fold signal-to-noise ratio enhancement. Fold change for each bin through the 1-500 cycles are plotted as a bar graph on the right.

FIG. 16A-16C. Signal amplification by one or two rounds of branching were compared to that from a linear amplification without branching. A 17-fold amplification for each branching round can be observed.

FIG. 17 determines how ACED showed high orthogonality by pairwise analysis of six extenders and metal strands.

FIG. 18 examines ACED mass cytometric analysis that was performed on Py2T cells that were undergoing EMT. Samples were analyzed without treatment or with 3, 5, or 7 days of TGFβ treatment. 30 EMT markers, including E-cadherin, vimentin, Zeb1, Snail/Slug, Smad2/3, and Smad4 were simultaneously analyzed to reveal the EMT states.

FIG. 19 shows how ACED enables detection of RNA targets in cells.

FIG. 20 depicts a scheme for multiplexed flow cytometry and flow sorting with TCE amplification.

FIG. 21A-21B. FIG. 21A depicts a scheme for GFP transfection and flow cytometry experiments. FIG. 21B examines the flow cytometry results of TCE-Flow vs secondary antibody staining. Signal to noise (GFP high/GFP low): 130 cycles of TCE: 333.35; 260 cycles TCE: 359.1; 2nd Ab: 97.80.

FIG. 22 shows a scheme for multiplexed immunoblotting with TCE amplification.

FIG. 23 depicts an immunoblot of GFP protein with TCE amplification. Left lane: protein ladder. Right lane: sample. Arrow indicates the target band. GFP is a 28 kD protein.

FIG. 24 shows a schematic depicting simultaneous amplification of RNA and protein signal using ISE in cells and tissue samples.

FIG. 25 is a broader schematic showing simultaneous amplification of Biomolecules using ISE in cells and tissue samples.

FIG. 26 depicts multiplexed protein and RNA imaging of cell line (Py2T cells) with or without TGFβ treatment. ISE validate the targets changes during epithelial to mesenchymal transition.

FIG. 27 depicts multiplexed protein and RNA imaging of PFA fixed tissue samples (mouse brain). 79-plex (43 RNA and 36 proteins) imaging of thin tissue (14 μm, mouse brain sagittal).

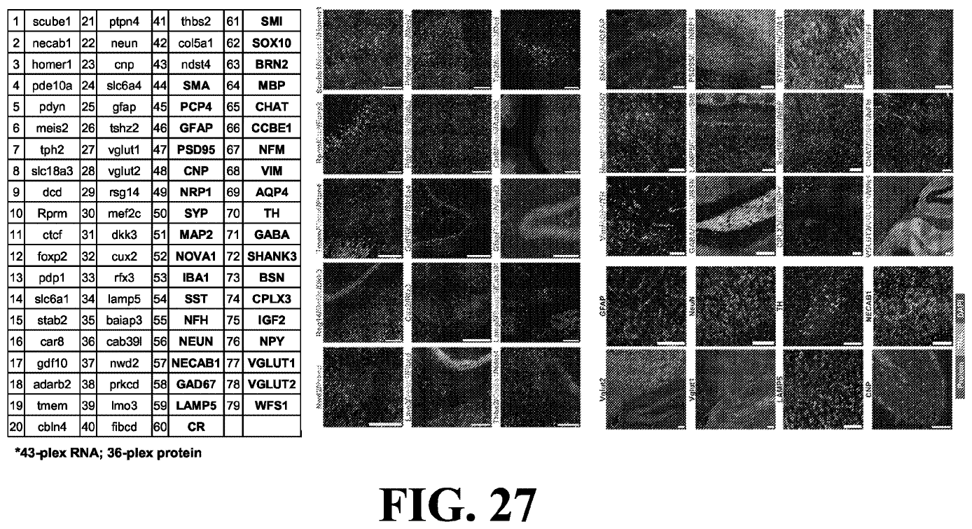

FIG. 28 shows 16 plex protein imaging in human cortical region fresh frozen samples. 16-plex imaging of thin tissue (12 μm, human cortical region).

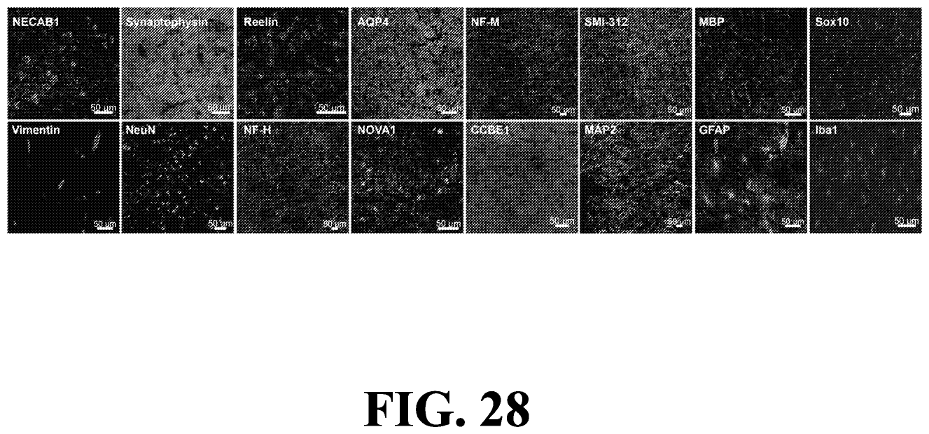

FIG. 29 shows 8-plex protein imaging of cleared thick mouse tissue (3×4×1 mm). ISE is compatible with various types of clearing protocols for thick tissue imaging.

FIG. 30 shows 4-plex protein imaging of cleared thick human tissue (3×3×1 mm, w/high-autofluorescence). Samples are photo-bleached by LED to reduce auto-fluorescence.

### DETAILED DESCRIPTION

The technology described herein relates to improvements in the ability to detect target molecules in situ in a manner that preserves information regarding the (often sub-cellular) location of such target molecules. More particularly, the technology described herein provides methods, compositions and kits for the labeling and detection of target ligand-binding molecules, wherein the labeling and detection are performed in situ on a cell or tissue sample contacted with the ligand-binding molecules. By way of example, the conjugation of oligonucleotides, and particularly oligonucleotides longer than about 40 nucleotides, to an antibody can influence the diffusion properties of the antibody and can contribute to non-specific binding. By generating the concatemeric oligonucleotides in situ, with the target-binding ligand already bound to its target, the methods described herein provide for significant signal amplification while avoiding such issues. The methods described herein further provide compositions, methods and kits that permit such labeling and/or detection of target-bound ligand-binding molecules in multiplex, and in a manner that permits signal from a plurality of different target-binding molecules to be distinguishably detected in multiplex.

In one embodiment, the technology described herein relates to the in situ generation of concatemers of nucleic acid sequence on a target ligand-binding molecule in which the repeated sequences provide hybridization sites for a plurality of labeled nucleic acid probes per target ligand binding molecule, thereby amplifying the signal related to the binding of target molecules in a cell or tissue sample by the target ligand-binding molecule. The plurality of hybridization sites provided by the concatemers can be thought of as “landing pads” for labeled probe molecules that hybridize, for example, to respective monomers in the concatemers. Where there are a plurality of such monomers per concatemer associated with the target ligand-binding molecule, the signal from each such target ligand-binding molecule is amplified, thereby facilitating the detection, quantitation and/or imaging of the target molecule as it occurs in situ.

In another embodiment, the in situ concatemer-generating approach lends itself well to multiplex labeling, detection and/or imaging in situ. As discussed in further detail below, one multiplex approach uses a set of different target ligand-binding molecules, each specific for a different target ligand of interest, and each conjugated to a different single-stranded oligonucleotide primer. When the respective primers are extended in multiplex to form concatemers of different repeat sequences by one or more of the methods described herein, the different repeats on the respective members of the set of target ligand-binding molecules provide different, highly selective binding sites or landing pads for a set of different, distinguishably-labeled probes that permit multiplex detection, quantitation and/or imaging of the members of a set of target molecules in situ.

The following describes various considerations involved in practicing the technology disclosed herein.

### Preparation of Cell or Tissue Samples

Samples, including cell and/or tissue samples, that are prepared for histology are fixed to avoid deterioration over subsequent staining and detection steps. The sample can be fixed as soon after collection as possible. There are many different types of fixatives known in the art. Exemplary fixatives include, but are not limited to, 4% paraformaldehyde, 4% formaldehyde, 10% neutral buffered formalin, Bouin's solution, methanol, acetone, glutaraldehyde, etc. The cell or tissue sample can be treated or processed so as to minimize nucleic acid degradation, where, for example, RNA is the target molecule. One of ordinary skill in the art will be able to determine the fixative best suited for the cell or tissue sample and technique to be performed. In some embodiments, the fixative that can be used is 4% paraformaldehyde.

In some embodiments, fixed cells can be in suspension, permitting, for example, the use of flow cytometry to sort or detect cells based on the presence or amount of one or more target ligands.

In some embodiments, fixed cells or tissue can be embedded in a medium facilitating, for example, sectioning for histology and/or imaging. Paraffin-embedding of fixed samples is well known in the art. Briefly, however, after a tissue sample has been fixed, it undergoes pre-embedding of paraffin to replace the water content of the sample with paraffin. Embedding in paraffin involves dehydration of tissues in increasing concentrations of alcohol, and then gradual replacement of alcohol by a paraffin solvent. Examples of a paraffin solvent include xylene. After pre-embedding, the sample is then embedded with melted paraffin using a mold, and hardened. Alternatives to paraffin wax include, but are not limited to epoxy, acrylic, agar, gelatin, and celloidin. One of ordinary skill in the art will be able to determine applicable embedding parameters.

After hardening, a paraffin-embedded tissue sample undergoes sectioning, wherein the sample is cut into thin slices, or sections, to be placed on a slide. These sections can generally be around 5 μm thick, though they can be thinner or thicker depending upon tissue type, target molecule, and label/label detection used, among other parameters. One of ordinary skill in the art will be able to determine how thick of a section is needed. Once the sections are cut, they are transferred to a warm water bath and placed on a charged slide. Slides are dried, allowing for the removal of excess wax.

In some embodiments, after the samples are placed onto a slide, the sample can undergo staining. Stains provide contrast to sections of tissue, making viewing structures of the sample easier. Exemplary stains include, but are not limited to alcian blue, aldehyde fuchsin, alkaline phosphatase, Bielshowsky stain, Congo red, crystal violet, eosin, Fontana-Masson, Giesmsa, Haematoxylin, Luna stain, Nissl, Period Acid Schiff (PAS), Red Oil 3, Reticulin stain, Sudan black, toluidine blue, and van Gieson. One of ordinary skill in the art will be able to determine what stain works best for the sample and technique to be performed. The term “staining” is also used in reference to the detection of particular target molecules, e.g., using target-binding ligands or molecules as described herein applied to cell or tissue samples. Additional details on histology can be found, for example, in Ross, M. H. et al. Histology: a text and atlas with correlated cell and molecular biology (7th ed.) Wolters Kluwer. (ISBN: 978-1451187427).

### Target-Binding Ligands

In some embodiments of any of the aspects, an oligonucleotide primer strand is attached to a target-binding ligand molecule.

As used herein, a “target-binding ligand” is a molecule or moiety that binds, e.g., specifically binds, to a target molecule of interest. The target-binding ligand can be a synthetic or natural molecule. A target-binding ligand can be a biomolecule, such as a polypeptide or a polynucleotide. In some embodiments, a target-binding ligand is a polypeptide. In some embodiments, a target-binding ligand is a protein or fragment thereof. Non-limiting examples of target-binding ligands include peptides, polypeptides, antibodies and antibody derivatives, oligonucleotides, aptamers and receptors.

In some embodiments of any of the aspects, the target-binding ligand binds to i.e., the target molecule is, a molecule selected from the non-limiting group of lipids, sugars, oligo- or poly-saccharides, amino acids, peptides or polypeptides, nucleosides, nucleotides, oligo- or poly-nucleotides, hormones, vitamins, small molecules, miRNAs, metabolites, and any combinations thereof.

In some embodiments of any of the aspects, the target-binding ligand binds a molecule that is DNA or RNA barcoded.

In some embodiments of any of the aspects, the target binding molecule is an antibody. The term “antibody” refers to a protein that includes at least one immunoglobulin variable domain or immunoglobulin variable domain sequence that binds a target molecule. For example, an antibody can include an immunoglobulin heavy (H) chain variable region (abbreviated herein as VH), and an immunoglobulin light (L) chain variable region (abbreviated herein as VL). In another example, an antibody includes two heavy (H) chain variable regions and two light (L) chain variable regions. An antibody can include the structural features of IgA, IgG, IgE, IgD, IgM (as well as subtypes thereof). The term antibody as used herein includes any of a number of different constructs using one or more antigen-binding domains or fragments of an antibody to mediate binding to a target molecule. Thus, in addition to a complete IgA, IgG, IgE, IgD or IgM antibody, an antibody includes, but is not limited to antigen-binding fragments of antibodies (e.g., single chain antibodies, Fab and sFab fragments, F(ab′)2, Fd fragments, Fv fragments, scFv, domain antibodies (dAb) (de Wildt et al., Eur J Immunol. 1996; 26(3):629-39) and nanobodies. An affibody, which uses a non-antibody scaffold to support a diverse target-binding domain, can also be used as a target-binding ligand in the methods, compositions and kits described herein.

As used herein, an “antigen-binding fragment” refers that portion of an antibody that is necessary and sufficient for binding to a given antigen. At a minimum, an antigen binding fragment of a conventional antibody will comprise six complementarity determining regions (CDRs) derived from the heavy and light chain polypeptides of an antibody arranged on a scaffold that permits them to selectively bind the antigen. A commonly used antigen-binding fragment includes the VH and VL domains of an antibody, which can be joined either via part of the constant domains of the heavy and light chains of an antibody, or, alternatively, by a linker, such as a peptide linker. Non-conventional antibodies, such as camelid and short antibodies have only heavy chain sequences, denoted, for example VHH. These can be used in a manner analogous to VH/VL-containing antigen-binding fragments. Non-limiting examples of antibody fragments encompassed by the term antigen-binding fragment include: (i) a Fab fragment, having VL, CL, VH and CH1 domains; (ii) a Fab′ fragment, which is a Fab fragment having one or more cysteine residues at the C-terminus of the CH1 domain; (iii) an Fd fragment having VH and CH1 domains; (iv) a Fd′ fragment having VH and CH1 domains and one or more cysteine residues at the C-terminus of the CH1 domain; (v) an Fv fragment having the VL and VH domains of a single arm of an antibody; (vi) a dAb fragment (Ward et al., Nature 341, 544-546 (1989)) which consists of a VH domain; (vii) F(ab′)2 fragments, a bivalent fragment including two Fab′ fragments linked by a disulphide bridge at the hinge region; (viii) single chain antibody molecules (e.g., single chain Fv; scFv) (Bird et al., Science 242:423-426 (1988); and Huston et al., PNAS (USA) 85:5879-5883 (1988)); (ix) “diabodies” with two antigen binding sites, comprising a heavy chain variable domain (VH) connected to a light chain variable domain (VL) in the same polypeptide chain (see, e.g., EP 404,097; WO 93/11161; and Hollinger et al., Proc. Natl. Acad. Sci. USA, 90:6444-6448 (1993)); and “linear antibodies” comprising a pair of tandem Fd segments (VH-CH1-VH-CH1) which, together with complementary light chain polypeptides, form a pair of antigen binding regions (Zapata et al. Protein Eng. 8(10):1057-1062 (1995); and U.S. Pat. No. 5,641,870). The molecules of Fv, scFv or diabody can be stabilized by incorporating disulfide bridges linking the VH and VL domains. Minibodies comprising a scFv fragment linked to a CH3 domain can also be obtained. Other examples of binding fragments are Fab′, which differs from Fab fragments by the addition of some residues at the carboxyl terminus of the CH1 domain of the heavy chain, including one or more cysteines of the hinge region of the antibody, and Fab′-SH, which is a Fab′ fragment in which the cysteine residue(s) of the constant domains carries a free thiol group.

### Oligonucleotides

Target-binding ligands as described herein include a single-stranded oligonucleotide primer that permits concatemer generation according to the methods described. Single-stranded oligonucleotide primers can be attached to target-binding ligands by any of various approaches known in the art. Note that while the single-stranded oligonucleotide can be DNA, RNA, or modified forms of either, for ease of reference and proof of principle, DNA oligonucleotides are used in the description and Examples that follow.

Oligonucleotides and oligonucleotide synthesis are well known in the art, and a number of different commercial sources provide custom oligonucleotide synthesis, such that an oligonucleotide with essentially any sequence can be readily obtained. Oligonucleotides are commercially available for custom design and use from Eurofins Genomics (Louisville, KY), ThermoFisher (Waltham, MA), Integrated DNA Technologies (Coralville, Iowa), TriLink Biotechnologies (San Diego, CA) and the like. Furthermore, it is known in the art how to generate oligonucleotides that include modified nucleosides and/or modified linkages, and oligonucleotides including any of a number of such modifications are also commercially available. To be clear, an oligonucleotide “primer” is an oligonucleotide that can be extended by a template-dependent nucleic acid polymerase when hybridized (e.g., via hydrogen-bonded base pairing) to a template nucleic acid molecule. A single-stranded extender template oligonucleotide as described herein has a 3′ modification or moiety (also referred to as a “stopper” moiety) that blocks or precludes template-dependent extension. Such a modification can also be referred to as a chain terminator, and includes, for example, a dideoxy nucleoside, 3′-CPR II CPG, 3′-phosphate CPG, 5′-Ome-dT-CE Phosphoramidite, 5′-amino-dT-CE Phosphoramidite, 2′3′-ddA-CE Phosphoramidite, 2′3′-ddC-CE Phosphoramidite, 2′3-ddG-CE Phosphoramidite, 2′3′-ddT-CE Phosphoramidite, 3′-dA-CPG, 3′-ddC-CPG, 3′-dC-CPG, 3′-dG-CPG, 3′-dT-CPG, 3′Spacer C3 CPG and the like. In some embodiments, oligonucleotides can also include, for example, nucleosides modified to include cross-linking moieties. The inclusion of a cross linking moiety, including but not limited to a photo cross-linking moiety, can permit, for example, the cross-linking of an oligonucleotide to a target sequence such that the oligonucleotide does not dissociate in subsequent processes, such as thermal cycling to generate concatemers. Thus, in some embodiments of any one of the aspects, an oligonucleotide strand comprises a photo-cross linking moiety, for example, a photo-cross linking moiety selected from the group consisting of 3-Cyanovinylcarbazole (CNVK) nucleotide; 5-bromo deoxycytosine; 5-iodo deoxycytosine; 5-bromo deoxyuridine (Bromo dU); 5-iodo deoxyuridine; and nucleotides comprising an aryl azide (AB-dUMP), benzophenone (BP-dUMP), perfluorinated aryl azide (FAB-dUMP) or diazirine (DB-dUMP), psoralen, 4-thio-dT (S4dT), and the like.

Oligonucleotides useful in the methods, compositions and kits described herein will generally be at least 9 nucleotides in length, but can vary from 5 to 100 or more nucleotides in length. Thus, an oligonucleotide useful in the methods, compositions or kits described herein can be, for example, between 5-100 nucleotides in length, between 5-95 nucleotides in length, between 5-90 nucleotides in length, between 5-85 nucleotides in length, between 5-80 nucleotides in length, between 5-75 nucleotides in length, between 5-70 nucleotides in length, between 5-65 nucleotides in length, between 5-60 nucleotides in length, between 5-55 nucleotides in length, between 5-50 nucleotides in length, between 5-45 nucleotides in length, between 5-40 nucleotides in length, between 5-35 nucleotides in length, between 5-30 nucleotides in length, between 5-25 nucleotides in length, between 5-20 nucleotides in length, between 5-15 nucleotides in length, between 5-10 nucleotides in length, between 5-9 nucleotides in length, between 9-100 nucleotides in length, between 9-95 nucleotides in length, between 9-90 nucleotides in length, between 9-85 nucleotides in length, between 9-80 nucleotides in length, between 9-75 nucleotides in length, between 9-70 nucleotides in length, between 9-65 nucleotides in length, between 9-60 nucleotides in length, between 9-55 nucleotides in length, between 9-50 nucleotides in length, between 9-45 nucleotides in length, between 9-40 nucleotides in length, between 9-35 nucleotides in length, between 9-30 nucleotides in length, between 9-25 nucleotides in length, between 9-20 nucleotides in length, between 9-15 nucleotides in length, between 10-100 nucleotides in length, between 10-90 nucleotides in length, between 10-80 nucleotides in length, between 10-70 nucleotides in length, between 10-60 nucleotides in length, between 10-50 nucleotides in length, between 10-40 nucleotides in length, between 10-30 nucleotides in length, between 10-20 nucleotides in length, between 11-100 nucleotides in length, between 11-90 nucleotides in length, between 11-80 nucleotides in length, between 11-70 nucleotides in length, between 11-60 nucleotides in length, between 11-50 nucleotides in length, between 11-40 nucleotides in length, between 11-30 nucleotides in length, between 11-20 nucleotides in length, between 12-100 nucleotides in length, between 12-90 nucleotides in length, between 12-80 nucleotides in length, between 12-70 nucleotides in length, between 12-60 nucleotides in length, between 12-50 nucleotides in length, between 12-40 nucleotides in length, between 12-30 nucleotides in length, between 12-20 nucleotides in length, between 13-100 nucleotides in length, between 13-90 nucleotides in length, between 13-80 nucleotides in length, between 13-80 nucleotides in length, between 13-70 nucleotides in length, between 13-60 nucleotides in length, between 13-50 nucleotides in length, between 13-40 nucleotides in length, between 13-30 nucleotides in length, between 13-20 nucleotides in length, between 14-100 nucleotides in length, between 14-90 nucleotides in length, between 14-80 nucleotides in length, between 14-70 nucleotides in length, between 14-60 nucleotides in length, between 14-50 nucleotides in length, between 14-40 nucleotides in length, between 14-30 nucleotides in length, between 14-20 nucleotides in length, between 15-100 nucleotides in length, between 15-90 nucleotides in length, between 15-80 nucleotides in length, between 15-70 nucleotides in length, between 15-60 nucleotides in length, between 15-50 nucleotides in length, between 15-40 nucleotides in length, between 15-30 nucleotides in length, or between 15-20 nucleotides in length.

Methods for the conjugation of oligonucleotides to antibodies are known in the art. See, e.g., Dugal-Tessier et al., J. Clin. Med. 10: 838 (2021) for a review discussing various methods. Kits are available for the conjugation of oligonucleotides to antibodies; see, e.g., Abcam Oligonucleotide Conjugation Kit (ab218260), which permits conjugation to the 5′ or 3′ end of oligos from 10 to 120 nucleotide long. Approaches appropriate for the conjugation of an oligonucleotide to a non-antibody target-binding ligand will vary depending up on the ligand, but are known to those of ordinary skill in the art. Single-stranded oligonucleotide primers are conjugated with target-binding ligand in a manner that permits template-directed nucleic acid polymerase extension from the 3′ end of the primer. While various approaches for primer conjugation can be used, it is therefore important that the method used keep the 3′ nucleotide open for extension. Examples include conjugation via linking moiety attached at or near the 5′ end of the oligonucleotide primer.

Included among oligonucleotides as described herein are nucleic acid probe molecules. In general, a nucleic acid probe molecule is an oligonucleotide that includes a label that is directly or indirectly detectable to provide a signal. Non-limiting examples of a label can include a fluorescent label or a metal, e.g. a lanthanide metal permitting detection in mass cytometry. Additional examples of detectable labels include, for example, isotopes, e.g., ³²P, ³⁵S, etc., quantum dots, organic dyes, polymer nanoparticles, metallic nanoparticles, and Raman dots, among others. Methods of the preparation of labeled oligonucleotide probes bearing any of a number of different detectable moieties are known to those of skill in the art. Multiple copies of a nucleic acid probe that binds to a sequence repeated in a concatemer produced as described herein can bind to such a concatemer such that each molecule of target bound by a target-binding ligand as described herein has multiple label moieties associated with it, greatly amplifying the signal, in situ, such that the presence, location and/or amount of a given target can be determined to as much as single-molecule sensitivity.

A wide variety of fluorescent reporter dyes are known in the art. Typically, the fluorophore is an aromatic or heteroaromatic compound and can be a pyrene, anthracene, naphthalene, acridine, stilbene, indole, benzindole, oxazole, thiazole, benzothiazole, cyanine, carbocyanine, salicylate, anthranilate, coumarin, fluorescein, rhodamine or other like compound.

Exemplary fluorophores include, but are not limited to, 1,5 IAEDANS; 1,8-ANS; 4-Methylumbelliferone; 5-carboxy-2,7-dichlorofluorescein; 5-Carboxyfluorescein (5-FAM); 5-Carboxynapthofluorescein (pH 10); 5-Carboxytetramethylrhodamine (5-TAMRA); 5-FAM (5-Carboxyfluorescein); 5-Hydroxy Tryptamine (HAT); 5-ROX (carboxy-X-rhodamine); 5-TAMRA (5-Carboxytetramethylrhodamine); 6-Carboxyrhodamine 6G; 6-CR 6G; 6-JOE; 7-Amino-4-methylcoumarin; 7-Aminoactinomycin D (7-AAD); 7-Hydroxy-4-methylcoumarin; 9-Amino-6-chloro-2-methoxyacridine; ABQ; Acid Fuchsin; ACMA (9-Amino-6-chloro-2-methoxyacridine); Acridine Orange; Acridine Red; Acridine Yellow; Acriflavin; Acriflavin Feulgen SITSA; Aequorin (Photoprotein); Alexa Fluor 350™; Alexa Fluor 430™M; Alexa Fluor 488™; Alexa Fluor 532™; Alexa Fluor 546™; Alexa Fluor 568™; Alexa Fluor 594™; Alexa Fluor 633™; Alexa Fluor 647™; Alexa Fluor 660™; Alexa Fluor 680™M; Alizarin Complexon; Alizarin Red; Allophycocyanin (APC); AMC, AMCA-S; AMCA (Aminomethylcoumarin); AMCA-X; Aminoactinomycin D; Aminocoumarin; Anilin Blue; Anthrocyl stearate; APC-Cy7; APTS; Astrazon Brilliant Red 4G; Astrazon Orange R; Astrazon Red 6B; Astrazon Yellow 7 GLL; Atabrine; ATTO-TAG™ CBQCA; ATTO-TAG™ FQ; Auramine; Aurophosphine G; Aurophosphine; BAO 9 (Bisaminophenyloxadiazole); BCECF (high pH); BCECF (low pH); Berberine Sulphate; Beta Lactamase; BFP blue shifted GFP (Y66H); BG-647; Bimane; Bisbenzamide; Blancophor FFG; Blancophor SV; BOBO™-1; BOBO™-3; Bodipy 492/515; Bodipy 493/503; Bodipy 500/510; Bodipy 505/515; Bodipy 530/550; Bodipy 542/563; Bodipy 558/568; Bodipy 564/570; Bodipy 576/589; Bodipy 581/591; Bodipy 630/650-X; Bodipy 650/665-X; Bodipy 665/676; Bodipy Fl; Bodipy FL ATP; Bodipy Fl-Ceramide; Bodipy R6G SE; Bodipy TMR; Bodipy TMR-X conjugate; Bodipy TMR-X, SE; Bodipy TR; Bodipy TR ATP; Bodipy TR-X SE; BO-PRO™-1; BO-PRO™-3; Brilliant Sulphoflavin FF; Calcein; Calcein Blue; Calcium Crimson™; Calcium Green; Calcium Green-1 Ca2+ Dye; Calcium Green-2 Ca2+; Calcium Green-5N Ca2+; Calcium Green-C18 Ca2+; Calcium Orange; Calcofluor White; Carboxy-X-rhodamine (5-ROX); Cascade Blue™; Cascade Yellow; Catecholamine; CFDA; CFP-Cyan Fluorescent Protein; Chlorophyll; Chromomycin A; Chromomycin A; CMFDA; Coelenterazine; Coelenterazine cp; Coelenterazine f; Coelenterazine fcp; Coelenterazine h; Coelenterazine hcp; Coelenterazine ip; Coelenterazine O; Coumarin Phalloidin; CPM Methylcoumarin; CTC; Cy2™; Cy3.1 8; Cy3.5™; Cy3™; Cy5.1 8; Cy5.5™; Cy5™; Cy7™; Cyan GFP; cyclic AMP Fluorosensor (FiCRhR); d2; Dabcyl; Dansyl; Dansyl Amine; Dansyl Cadaverine; Dansyl Chloride; Dansyl DHPE; Dansyl fluoride; DAPI; Dapoxyl; Dapoxyl 2; Dapoxyl 3; DCFDA; DCFH (Dichlorodihydrofluorescein Diacetate); DDAO; DHR (Dihydrorhodamine 123); Di-4-ANEPPS; Di-8-ANEPPS (non-ratio); DiA (4-Di-16-ASP); DIDS; Dihydrorhodamine 123 (DHR); DiO (DiOC18(3)); DiR; DiR (DiIC18(7)); Dopamine; DsRed; DTAF; DY-630-NHS; DY-635-NHS; EBFP; ECFP; EGFP; ELF 97; Eosin; Erythrosin; Erythrosin ITC; Ethidium homodimer-1 (EthD-1); Euchrysin; Europium (III) chloride; Europium; EYFP; Fast Blue; FDA; Feulgen (Pararosaniline); FITC; FL-645; Flazo Orange; Fluo-3; Fluo-4; Fluorescein Diacetate; Fluoro-Emerald; Fluoro-Gold (Hydroxystilbamidine); Fluor-Ruby; FluorX; FM 1-43™; FM 4-46; Fura Red™ (high pH); Fura-2, high calcium; Fura-2, low calcium; Genacryl Brilliant Red B; Genacryl Brilliant Yellow 10GF; Genacryl Pink 3G; Genacryl Yellow 5GF; GFP (S65T); GFP red shifted (rsGFP); GFP wild type, non-UV excitation (wtGFP); GFP wild type, UV excitation (wtGFP); GFPuv; Gloxalic Acid; Granular Blue; Haematoporphyrin; Hoechst 33258; Hoechst 33342; Hoechst 34580; HPTS; Hydroxycoumarin; Hydroxystilbamidine (FluoroGold); Hydroxytryptamine; Indodicarbocyanine (DiD); Indotricarbocyanine (DiR); Intrawhite Cf; JC-1; JO-JO-1; JO-PRO-1; LaserPro; Laurodan; LDS 751; Leucophor PAF; Leucophor SF; Leucophor WS; Lissamine Rhodamine; Lissamine Rhodamine B; LOLO-1; LO-PRO-1; Lucifer Yellow; Mag Green; Magdala Red (Phloxin B); Magnesium Green; Magnesium Orange; Malachite Green; Marina Blue; Maxilon Brilliant Flavin 10 GFF; Maxilon Brilliant Flavin 8 GFF; Merocyanin; Methoxycoumarin; Mitotracker Green FM; Mitotracker Orange; Mitotracker Red; Mitramycin; Monobromobimane; Monobromobimane (mBBr-GSH); Monochlorobimane; MPS (Methyl Green Pyronine Stilbene); NBD; NBD Amine; Nile Red; Nitrobenzoxadidole; Noradrenaline; Nuclear Fast Red; Nuclear Yellow; Nylosan Brilliant Iavin E8G; Oregon Green™; Oregon Green 488-X; Oregon Green™ 488; Oregon Green™ 500; Oregon Green™ 514; Pacific Blue; Pararosaniline (Feulgen); PE-Cy5; PE-Cy7; PerCP; PerCP-Cy5.5; PE-TexasRed (Red 613); Phloxin B (Magdala Red); Phorwite AR; Phorwite BKL; Phorwite Rev; Phorwite RPA; Phosphine 3R; PhotoResist; Phycoerythrin B [PE]; Phycoerythrin R [PE]; PKH26; PKH67; PMIA; Pontochrome Blue Black; POPO-1; POPO-3; PO-PRO-1; PO-PRO-3; Primuline; Procion Yellow; Propidium Iodid (PI); PyMPO; Pyrene; Pyronine; Pyronine B; Pyrozal Brilliant Flavin 7GF; QSY 7; Quinacrine Mustard; Resorufin; RH 414; Rhod-2; Rhodamine; Rhodamine 110; Rhodamine 123; Rhodamine 5 GLD; Rhodamine 6G; Rhodamine B 540; Rhodamine B 200; Rhodamine B extra; Rhodamine BB; Rhodamine BG; Rhodamine Green; Rhodamine Phallicidine; Rhodamine Phalloidine; Rhodamine Red; Rhodamine WT; Rose Bengal; R-phycoerythrin (PE); red shifted GFP (rsGFP, S65T); S65A; S65C; S65L; S65T; Sapphire GFP; Serotonin; Sevron Brilliant Red 2B; Sevron Brilliant Red 4G; Sevron Brilliant Red B; Sevron Orange; Sevron Yellow L; sgBFP™; sgBFP™ (super glow BFP); sgGFP™; sgGFP™ (super glow GFP); SITS; SITS (Primuline); SITS (Stilbene Isothiosulphonic Acid); SPQ (6-methoxy-N-(3-sulfopropyl)-quinolinium); Stilbene; Sulphorhodamine B can C; Sulphorhodamine G Extra; Tetracycline; Tetramethylrhodamine; Texas Red™; Texas Red-X™ conjugate; Thiadicarbocyanine (DiSC3); Thiazine Red R; Thiazole Orange; Thioflavin 5; Thioflavin S; Thioflavin TCN; Thiolyte; Thiozole Orange; Tinopol CBS (Calcofluor White); TMR; TO-PRO-1; TO-PRO-3; TO-PRO-5; TOTO-1; TOTO-3; TriColor (PE-Cy5); TRITC (TetramethylRhodamineIsoThioCyanate); True Blue; TruRed; Ultralite; Uranine B; Uvitex SFC; wt GFP; WW 781; XL665; X-Rhodamine; XRITC; Xylene Orange; Y66F; Y66H; Y66W; Yellow GFP; YFP; YO-PRO-1; YO-PRO-3; YOYO-1; and YOYO-3. Methods for fluorophore labeling of nucleic acids are known in the art. For multiplex approaches, probes labeled with fluorophores with distinguishable excitation and/or emission spectra can be used.

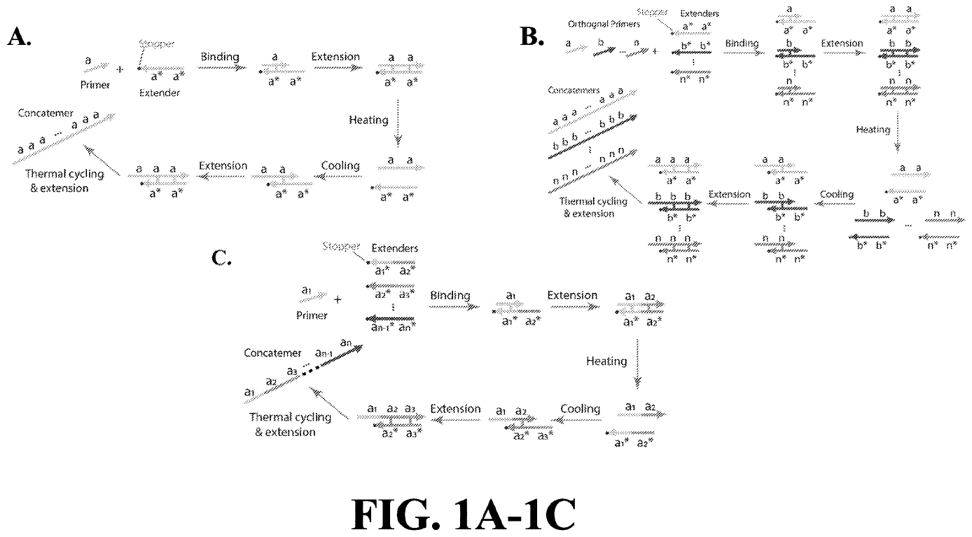

Several embodiments of concatemer-generating approaches described herein are illustrated in FIG. 1.

Single-stranded oligonucleotide primers will comprise, or consist of, a unit sequence that will be repeated in a concatemer generated using the methods described herein. The concatemer repeat element is represented in FIG. 1A as “a”. In effect, the actual concatemer repeat sequences will be determined by the sequence of the single-stranded extender template as described herein. That is, an oligonucleotide primer conjugated to a target-binding ligand can include 5′ sequence in addition to the sequence that becomes the concatemer repeat sequence, but the 3′ portion of the original oligonucleotide primer that is complementary to the sequence repeated in a head-to-tail manner in the single-stranded extender template will determine the repeat sequence. This may be illuminated by reference to, for example, FIG. 1A, which shows oligonucleotide Primer “a”, and Extender “a*a*,” in which a* is the complement of a. The optional inclusion of additional sequence (not shown) at the 5′ end of the Primer that is not necessarily complementary to a* would act as essentially a linker that does not get copied in the concatemer based on the extender template's copies of a*. Single-stranded extender templates are discussed in further detail herein below, and demonstrated in the Examples.

As discussed further herein below, an advantage of the methods, compositions and kits described herein is the ability to generate concatemer-labeled target-binding ligands (and ultimately to detect them) in multiplex. The length of the concatemer repeat sequence directly influences the degree of multiplex that can be achieved in the methods, compositions and kits described herein. Generally, the longer the repeat unit, the greater the degree of multiplexing one can achieve with orthogonal primers of that length. For example, for a repeat unit length of 9 nucleotides, a maximum of 93 orthogonal primers are possible. For a repeat unit length of 10 nt, the maximum increases to 170 primers. For a repeat unit length of 11 nt, the maximum is 315. For 12 nt, the maximum is 585. For 13 nt, the maximum is 1092. For 14 nt, the maximum is 2048, and for 15 nt, the maximum is 3855. For multiplex applications, it is preferred, but not absolutely required that the concatemer repeat unit on each target-binding ligand be the same length. Similar repeat unit lengths can provide, among other things, similar efficiencies for cycles of extender annealing and primer extension.

In one embodiment, the target molecule can itself be an RNA molecule, including but not limited to an mRNA molecule. The hybridization of one or more single-stranded oligonucleotides comprising a 5′ region complementary to a sequence on the target mRNA and a 3′ region comprising at least one sequence to be extended as a concatemer provides an initiation site for the generation of a concatemer using single-stranded extender template oligonucleotides as described herein. In this manner, a nucleic acid comprising this type of 5′ and 3′ regions can serve as a target-binding ligand molecule as that term is used herein. Where the target molecule is an RNA, the single-stranded target-binding ligand oligonucleotide can be hybridized to its RNA target in situ, and then cross-linked so as to maintain its association with the target RNA through subsequent steps of concatemer generation. Cross-linking can be achieved, e.g., via the incorporation of photo cross-linking agents as described herein or as known in the art into the single-stranded target-binding ligand oligonucleotide (and preferably only into the portion of the oligonucleotide that hybridizes to target RNA). After hybridization and removal of non-hybridized oligonucleotide, the sample is irradiated with UV light to cross-link the oligonucleotide to its RNA target.

In one embodiment, the target molecule can itself be a DNA molecule, or a sequence on such a molecule, including, but not limited to a chromosomal or episomal DNA sequence. The hybridization of one or more single-stranded oligonucleotides comprising a 5′ region complementary to a sequence on the target DNA and a 3′ region comprising at least one sequence to be extended as a concatemer provides an initiation site for the generation of a concatemer using extender oligonucleotides as described herein. In this manner, a nucleic acid comprising this type of 5′ and 3′ regions can serve as a target-binding ligand molecule as that term is used herein. Where the target molecule is a DNA molecule of a given sequence, the single-stranded target-binding ligand oligonucleotide can be hybridized to its DNA target in situ via target-complementary sequence located 5′ of the 3′ sequence to be extended as a concatemer repeat element, and then (after removal of non-hybridized oligonucleotide) cross-linked, e.g., as known in the art or as described herein, so as to maintain its association with the target DNA through subsequent steps of concatemer generation.

As noted, an important advantage of the methods, compositions and kits described herein is the ready ability to perform the concatemer-generating steps in multiplex, such that a plurality of different target-binding ligands have different concatemeric probe landing pads generated in situ, in multiplex. This is illustrated, for example, in FIG. 1B, wherein orthogonal primers with repeat sequences “a,” “b” . . . “n” are extended using single-stranded extender templates with at least two repeats or copies of complementary sequences “a*,” “b*,” . . . “n*.” Once generated, the different concatemers provide landing pads for orthogonal complementary probes that can detect the various target ligands, also in multiplex. Various approaches to detecting and distinguishing probes in multiplex are known in the art and/or discussed elsewhere herein.

Target-binding ligands bearing oligonucleotide primers as described herein can be contacted with a cell or tissue sample preparation according to methods known in the art. Where, for example, the target-binding ligand is an antibody or antigen-binding fragment thereof, methods widely applied in immunohistochemistry can be used to stain the sample for detection of the given target ligand. Thus, the methods for contacting the cell or tissue sample with the target-binding ligand can parallel those used with, e.g., fluorescently labeled antibodies or antibody fragments. Where performed in multiplex, the contacting or staining can comprise the addition of a set of target-binding ligands, each comprising a different oligonucleotide primer. In instances where the target-binding ligand is not an antibody or antigen-binding fragment thereof, methods for contacting a cell or tissue preparation with the target-binding ligand can be adapted from those known in the art for the given ligand. In particular, where the target molecule is a DNA or RNA molecule comprising a given sequence, conditions as used, e.g., for in situ hybridization can be used to permit binding of the target-binding ligand oligonucleotide(s) to the target sequence(s). In some embodiments, it can be beneficial to remove unbound target-binding ligand prior to subsequent concatemer-generating or detection steps. This can be achieved, for example, by removal of target-binding ligand-containing solution from the cell or tissue sample, followed by one or more rinsing steps in an appropriate solution lacking the target-binding ligand.

### In Situ Generation of Concatemers

In one embodiment, described herein is a method of labeling a target-binding ligand with a concatemer that provides sites for the binding of multiple copies of a labeled nucleic acid probe complementary to a concatemer repeat sequence. The following describes the generation of concatemers on target-binding ligands, and can be performed in situ, for example, with the target-binding ligand bound to its target molecule in a cell or tissue sample. The methods generally use oligonucleotides conjugated to target-binding ligands, and single-stranded extender templates, with repeated cycles of annealing, polymerase extension and thermal strand separation, wherein each cycle adds another concatemeric repeat to the oligonucleotide conjugated to the target-binding ligand. In one embodiment, such a method of labeling a target-binding ligand comprises (a) providing a conjugate of the target-binding ligand and a first oligonucleotide primer; (b) in a reaction mixture, contacting the conjugated first oligonucleotide primer with a first single-stranded extender template and a nucleic acid polymerase enzyme under conditions permitting hybridization and extension of the first oligonucleotide primer using the first single-stranded extender template; (c) heating the reaction mixture of step (b) to separate the first single-stranded extender template from extended first oligonucleotide primer produced in step (b); and (d) repeating steps (b) and (c) at least once, thereby generating a concatemer comprising repeats of oligonucleotide primer sequence, conjugated to the target-binding ligand.

Conjugates of target-binding ligand and an oligonucleotide primer are discussed above. The exact makeup of reaction mixtures effective for the template-mediated extension of a primer, in terms of extender template and nucleotide concentrations, buffer, salts, divalent cations and other co-factors and their concentrations will depend upon the particular polymerase enzyme used, as will, for example, temperatures for the extension reactions. One of ordinary skill in the art can adjust these parameters for a given polymerase enzyme.

The melting temperature, Tₘ, for a nucleic acid duplex is a measure of thermal stability of the duplex, and defined as the temperature at which half of the DNA strands are in the single-stranded or dissociated state. The Tₘ for any given nucleic acid duplex is determined by parameters including its length, nucleotide composition, nucleic acid concentration and salt conditions in a given reaction. A rule of thumb formula for calculating Tm for short oligonucleotides is Tₘ=2×(A+T)+4×(G+C), but other formulae can provide more accurate estimates, e.g., Tₘ=81.5+0.41(% G+C)−675/N, where N is the total number of bases. Optimal annealing temperatures for primer extension reactions can be determined empirically by one of ordinary skill in the art, but will generally be about 5° C. below the Tₘ for a given primer/template duplex. When performed in multiplex, it is preferred that the annealing temperature be about 5 oC below the Tₘ for the primer/template duplex with the lowest Tₘ, but that primers be designed to minimize Tₘ differences. Primer design for multiplex primer extension reactions is well established in the art, and takes into account additional parameters, including, but not limited to minimizing primer-dimer formation (wherein one primer has sufficient complementarity to another primer in the same reaction to permit extension using the other primer as template) and minimizing off-target extension or amplification. Software tools for multiplex primer design are known in the art and include, for example, OligoPerfect™ Primer Designer, available from ThermoFisher Scientific, among others.

Thus, in a given concatemer-generating regimen, the oligonucleotide primer conjugated with a target-binding ligand is contacted in situ, in a reaction mixture appropriate for the polymerase enzyme used, with a single-stranded extender template oligonucleotide at a temperature, determined, for example, on the basis of Tₘ for the primer-template hybridized duplex. This permits annealing of single-stranded extender template to the primer, and extension of the annealed primer by the chosen polymerase enzyme when incubated at the extension temperature for the polymerase. Cycles of heating to dissociate the extended strand from the single-stranded extender template, cooling to permit annealing of single-stranded extender template and incubation at the extension temperature for the polymerase generate a concatemer with repeats of the oligonucleotide primer sequence defined by the single-stranded extender template. When performed in multiplex, concatemers of different, orthogonal repeat sequences on different target-binding ligands bound to their respective targets in situ are generated. Subsequent detection based on the hybridization of distinguishably-labeled probes to the concatemers permits detection, quantitation and/or imaging of the target molecules in situ.

### Single-Stranded Extender Templates

The use of single-stranded extender templates is central to the methods, compositions and kits described herein. Single-stranded extender templates useful in the methods, compositions and kits described herein comprise a concatemer of at least two head-to-tail copies of a sequence complementary to the oligonucleotide primer conjugated to the target-binding ligand. The single-stranded extender template further comprises a 3′ blocking or chain-terminating modification such that the single-stranded extender template is not itself extendable by the polymerase used to generate concatemers. The design of the single-stranded extender template provides for a method in which one copy of the concatemer repeat sequence complement hybridizes to the oligonucleotide conjugated to the target-binding ligand, and the at least one additional copy of the concatemer repeat sequence complement provides the template for the extension of the oligonucleotide conjugated to the target ligand. As a representative illustration, see again, e.g., FIG. 1A (Extender a*a*, with a 3′ “stopper” or chain terminator). Because the single-stranded extender template must include sequence complementary to the desired repeat sequence of a concatemer to be generated, the sequence of the single-stranded extender template is dictated by that desired repeat sequence. As discussed above in regard to primer design, the sequence of the desired concatemeric repeats is dictated by parameters permitting multiplex target detection while minimizing primer:primer interactions and off-target hybridization.

Where performed in multiplex, a set of different single-stranded extender templates, orthogonal to a set of oligonucleotide primers conjugated to respective members of a set of different target-binding ligands can be used. As with single-stranded extender templates used singly, each single-stranded extender template in a set comprises a concatemer of at least two head-to-tail copies of a sequence complementary to a different oligonucleotide primer conjugated to a target-binding ligand, e.g., as described herein. Each single-stranded extender template in such a set also comprises the 3′ blocking or chain-terminating modification that precludes polymerase extension of the extender template. Such a multiplex approach, illustrated, for example, in FIG. 1B (Extenders comprising a*a*, b*b* and n*n*, each with 3′ stoppers), generates concatemers with different repeat unit sequences on the respective members of the set of target-binding ligands.

The polymerase used to extend oligonucleotide primers and generate concatemers can be any of a number of template-dependent nucleic acid polymerases. In one embodiment, the polymerase is thermostable, such that it can withstand heating to a temperature and for a time sufficient to denature or dissociate double-stranded nucleic acids, retaining template-dependent polymerization activity when the reaction mixture is cooled to a temperature permitting annealing of extender template(s) to oligonucleotide primer(s) and primer extension. Different thermostable polymerases have different reaction buffer and extension temperature optima; these parameters are known to those of ordinary skill in the art and/or described in product literature for given polymerases. Non-limiting examples of thermostable polymerases useful in the methods, compositions and kits described herein include the following. Examples of polymerases that can be used in the methods described herein include but are not limited to: Standard Taq DNA polymerase (Cat. No. 10342053, Invitrogen, Carlsbad, CA), Platinum II Taq Hot-Start DNA Polymerase (Cat. No. 14966001, Invitrogen, Carlsbad, CA), Platinum SuperFi II DNA Polymerase (Cat. No. 12361010, Invitrogen, Carlsbad, CA), USB™ CycleSeq™ Thermostable DNA Polymerase (Cat. No. 792001000UN, Applied Biosystems, Waltham, MA); Taq DNA Polymerase (Cat. No. EP0402, ThermoScientific, Waltham, MA); HoTaq DNA Polymerase (HT-200, McLab, San Francisco, CA); I-5 Hi-Fi DNA Polymerase (PDP-100, McLab, San Francisco, CA); I-5 Hotstart DNA Polymerase (I5HD-100, McLab, San Francisco, CA); DNA polymerase, Thermotoga neapolitana (DPTN-100, McLab, San Francisco, CA); Pfu DNA Polymerase (AD-200, McLab, San Franscisco, CA); Pfu DNA Polymerase (Cat. No. 600135, Agilent Technologies, Wood Dale, IL); PfuTurbo DNA Polymerase (Cat. No. 600252, Agilent Technologies, Wood Dale, IL) and the like. One of ordinary skill in the art can identify additional polymerases that would function in the methods, compositions and kits described herein and can adjust reaction conditions as may be needed for any given polymerase.

### Branched Concatemers

An option for further increasing the number of probe-binding sites on a target-binding ligand is to introduce branching such that initial, linear concatemers on a target-binding ligand provide sites for the generation of additional concatemers that branch off of the initial linear concatemers. It is important to note that in some embodiments, branching can be performed more than once, using earlier branches as sites for the generation of additional concatemers, with each round of branching multiplying the number of concatemer repeats and thus the number of potential probe-binding sites on a given target-binding ligand. In some embodiments, the branches are generated in situ on a linear concatemer generated as described herein. In other embodiments, pre-formed concatemers are added to a reaction mixture that hybridize to repeats in a concatemer on a target ligand. These approaches are discussed further herein below.

In one embodiment, for the generation of branched concatemers on a target-binding ligand in situ, after the in situ generation of a linear concatemer as described herein, a second oligonucleotide primer and a second single-stranded extender template are added to the reaction mixture. Considerations for design of the second oligonucleotide primer for uniplex and for multiplex branched concatemer generation parallel those for design of the first oligonucleotide primer in terms of avoiding cross-talk, primer dimers and off-target hybridization. As with the first oligonucleotide primer, any of a number of different software packages can assist in such design.

In this embodiment, the second oligonucleotide primer comprises, in 5′ to 3′ order, sequence complementary to the first oligonucleotide primer, and sequence complementary to the second single-stranded extender template, wherein the second single-stranded extender template comprises a 3′ chain terminator. Molecules of the second oligonucleotide primer are permitted to anneal, via its sequence complementary to the first oligonucleotide primer, to at least one, and preferably a plurality of concatemeric repeats in the first extended concatemeric oligonucleotide conjugated to the target-binding ligand.

The sequence complementary to the first oligonucleotide primer can include a (photo) cross-linking moiety, such that after annealing, irradiation or other appropriate treatment of the reaction mixture will cross-link the second oligonucleotide primer to the extended first oligonucleotide primer so the second oligonucleotide primer remains associated with the extended first oligonucleotide primer through subsequent steps of thermal strand separation.

For concatemer generation, the second single-stranded extender template anneals to its complementary sequence on the second oligonucleotide primer, and the second oligonucleotide primer is extended, thereby adding a new copy of the second concatemeric repeat to the new branch from the first concatemer. Repeated cycling of thermal strand separation and second single-stranded template-directed primer extension generate concatemers comprising repeats including sequence in the second oligonucleotide primer. Where a plurality of second oligonucleotide primers hybridize to a plurality of concatemer repeats on the first extended concatemeric oligonucleotide, the first extended oligonucleotide provides, in effect, a stem with many branches, each branch including multiple repeats of the second concatemer repeat sequence. Additional rounds of branching with third, fourth, fifth or more oligonucleotide primers and their orthogonal single-stranded extender templates analogous in design to the second oligonucleotide primer/second single-stranded extender template can provide further amplification of the number of probe-binding sites associated with a given target-binding ligand.

An alternative branching approach generates concatemers of a second repeat sequence that comprise, e.g., at their 5′ ends, a sequence complementary to the first concatemer repeat. These second concatemeric molecules are then permitted to hybridize to repeats in the first target-binding ligand associated concatemer via that complementary sequence to provide the branched concatemers. The sequence complementary to the first concatemeric repeat can include a (photo) cross-linking moiety permitting cross-linking of the second concatemers to the first concatemer. It is contemplated that the second concatemeric molecules could themselves include branches with third, fourth, fifth or more repeat elements to further amplify the number of probe-binding sites associated with a given target-binding ligand.

Either of the alternative branching approaches discussed above can be used in multiplex reactions as described herein.

### Ordered Extension and Nucleic Acid Generation

In another aspect, the single-stranded extender template approach can permit the generation of a nucleic acid having a desired sequence or ordered set of sequence elements. In this aspect, a set of different single-stranded extender elements can be used, each comprising sequence complementary to a prior extension element and a new extension element template. Repeated cycles of thermal strand separation and extension using successive single-stranded extension templates permits the generation of the nucleic acid having the desired sequence or ordered set of sequence elements.

In one embodiment, illustrated, for example, in FIG. 1C, an ordered extension approach to generating a nucleic acid molecule of a desired sequence comprises providing a first oligonucleotide primer, and, in a reaction mixture, contacting the first oligonucleotide primer with a first single-stranded extender template and a nucleic acid polymerase enzyme under conditions permitting hybridization and extension of the first oligonucleotide primer using the first single-stranded extender template. The polymerase can be a thermostable polymerase, e.g., as described herein or known in the art. The first single-stranded extender template comprises, in a 5′ to 3′ direction or orientation, a first extension template element, a sequence complementary to the first oligonucleotide primer, and a 3′ chain terminator. This step extends the first oligonucleotide primer by the complement of the first extension template element. It is noted that this ordered extension approach can be performed in which successive single-stranded extender templates are added at successive steps, or, in the alternative, a full set of single-stranded extender templates can be added at once, with successive thermal cycling building the ordered extension(s) in a single pot reaction based on specific hybridization of the appropriate single-stranded extender to each successively added extension.

Following the extension step, the mixture is heated to separate strands, and a second single-stranded extender template is contacted with the extended oligonucleotide primer generated in the first extension step, under conditions permitting hybridization and extension of the first extended oligonucleotide primer using the second single-stranded extender template. The second single-stranded extender template comprises, in a 5′ to 3′ direction or orientation, a second extension template element, a copy of the first extension template element, and a 3′ chain terminator. This step adds sequence complementary to the second extension template element to the first extended oligonucleotide primer. Successive rounds of strand separation and contacting with successive single-stranded extender templates, each including, in a 5′ to 3′ direction or orientation, a new extension template element, a copy of the previous extension template element, and a 3′ chain terminator can be performed. Design of the successive single-stranded extender templates permits the assembly of a nucleic acid molecule of essentially any desired sequence, comprised of sequence elements complementary to the successive extension template elements. Put differently, the ordered extension approach can be used to generate a nucleic acid molecule comprising, in order, first oligonucleotide primer sequence, then complement of each respective extension template element. If desired, among other uses, this approach can be used for barcoding a surface, a target-binding ligand, or other moiety to which the first oligonucleotide is conjugated.

The ordered extension approach can include, if desired, including a single-stranded extension template as described herein above for concatemer generation to the reaction such that one or more extension elements is repeated in concatemeric form in the generated nucleic acid molecule. This can be included before or after one or more rounds of single-stranded extender template switching that introduce new sequence elements to the growing extended oligonucleotide. Repeats added in this manner can thus provide for signal amplification as described herein.

The ordered extension approach can also be performed in multiplex. Primer design considerations are analogous to those for designing primers for multiplex concatemer-generating regimens described herein.

The ordered extension approach can be performed with the first oligonucleotide primer in solution, or on or conjugated to a surface upon which one wishes to build a nucleic acid molecule. The method can also be performed wherein the oligonucleotide primer is conjugated to a target-binding ligand as described herein. The method can also be performed in contact with a cell or tissue sample, such that the nucleic acid molecule is generated in situ in association with the cell or tissue sample, e.g., in association with a target molecule in the cell or tissue sample bound by a target-binding ligand.

The ordered extension approach can further comprise contacting the generated nucleic acid molecule with a labeled nucleic acid probe comprising sequence of one or more of the extension template elements. Detection of the label associated with the probe can provide information regarding the presence, amount and/or location of a target associated with the first oligonucleotide primer.

In an alternative approach, a method of adding a long DNA strand to extend only once, instead of short ones with multiple rounds of thermal cycling is also contemplated. The long DNA strand of one-step extension is a variation of the schematic of FIG. 1C—consider a2* to be repeats for imager binding. For the one-step extension scheme, instead of using heat to melt off the long DNA strand to allow imager binding and CNVK crosslinking, the alternatives would be to incorporate uracil bases in the long DNA strand and use uracyl DNA glycosylase enzyme (e.g., uracil DNA glycosylase; M0280S, New England Biolabs) digestion to fragment the long DNA strand after one-step extension to permit imager binding and CNVK crosslinking.

### Detection of Target Molecules In Situ

Among other uses, the compositions, methods and kits described herein can be applied to the detection of target molecules in situ. Once concatemeric sequences are generated on a target-binding ligand bound to its target in situ, a labeled nucleic acid probe (or set of probes when multiplex detection is performed) can be contacted with and permitted to hybridize with concatemeric repeats on the target-binding ligand. Label associated with the target-binding ligand in this manner can then be detected in a manner appropriate for the given label.

Where multiple targets are detected in a single assay, the label on individual probes should be distinguishable from others used in the same assay. Where the labels are, for example, fluorescent, they should have excitation and/or emission spectra that permit the user to distinguish one from the other in the same assay. It is contemplated that repeated rounds of detection using different wavelengths for excitation of different fluorophores with non-overlapping excitation spectra can be performed. Similarly, detection of non-overlapping emission spectra from different fluorophores can be used to detect a plurality of different fluorophores associated with a plurality of different targets. It is also contemplated that a first round of fluorescently-labeled probes can be applied and detected, followed by photo-bleaching of the fluorophore, before application of a second round of different probes labeled with one or more of the same fluorophores as the first round.

Where mass cytometry is used for detection, each of the lanthanide metals can be distinguished by time-of-flight mass spectrum, such that probes bearing different metal ion labels can permit multiplex detection of different target molecules in the same assay.

Given the high degree of signal amplification provided by the concatemeric repeats on the target-binding ligands, detection down to as much as single-molecule sensitivity is achievable, in multiplex, using the methods described herein.

### Mass Cytometry

Mass cytometry, also termed cytometry by Time-Of-Flight (CyTOF®), provides a tool for high-dimensional and high-throughput single-cell analyses. First introduced in 2009 (Bandura et al., Anal. Chem. 81: 6813-6822 (2009)), mass cytometry has become widely used in the analysis of immune cell function/activation and other processes due to its high-parameter capabilities. By using metal ion labels in place of fluorescent labels generally used in fluorescent-based flow cytometry, mass cytometry overcomes the problem of overlapping emission spectra that reduces the number of different targets or parameters that can be analyzed in a single assay.

Methods, applications and considerations for performing mass cytometry are reviewed, for example by Iyer et al., Front. Immunol. 13, Article 815828 (2022). Briefly, in mass cytometry, cells are generally incubated with a mixture of antibodies tagged with a unique non-radioactive heavy metal (most often, lanthanide) isotope. Single-cell suspensions are nebulized in a manner in which each droplet contains a single cell. Individual cells pass through argon (Ar) plasma, which atomizes and ionizes the sample. This process converts each cell into a cloud containing ions of the elements present in or on that cell. A high-pass optic (quadrupole) removes the low-mass—mainly biologic—ions from each cloud (i.e., those with mass below 75 Da), resulting in a cloud containing only those ions derived from the isotope-conjugated probes. In the Time of Flight (TOF) chamber, the ions are separated by mass-to-charge ratio. Upon encountering the detector, the ion counts are amplified and converted into electrical signals. Higher numbers of parameters corresponding to different targets are theoretically possible, but in current practice, about 60 different parameters are distinguishable in a mass cytometry panel. When adapted to use concatemer-based labeling/signal amplification methods as described herein, high multiplex, high sensitivity detection of a large number of even low-abundance targets can be performed.

As noted herein above, the concatemer-based signal amplification approaches described herein can be readily applied to other methods of biomolecule target detection or analysis. Non-limiting examples include, in addition to flow cytometry, mass cytometry and e.g., western blotting, methods including but not limited to imaging mass cytometry (see, e.g., Giesen et al., Nat. Methods 11: 417-422 (2014), see, also, the Hyperion+TM Imaging System (Fluidigm, Inc.; see the world wide web at fluidigm.com/products-services/technologies/imaging-mass-cytometry), multiplexed ion beam imaging (MIBI; see, e.g., Angelo et al. Nat. Med. 20: 436-442 (2014)), and multiplexed ion beam imaging by time of flight (MIBI TOF; see, e.g., Keren et al., Sci. Adv. 5: eaax5851 (2019)), among others.

It is noted that the concatemer-based methods and compositions described herein, and the cross-linking of probes with nucleic acids associated with target-binding ligands can be applied to or combined with other approaches for detecting target molecules. For example, the concatemer approaches described herein can be used to further amplify signal in RNAScope™ (see, e.g., Wang, H. et al. (2015). Multiplex Fluorescent RNA In Situ Hybridization Via RNAscope. In: Hauptmann, G. (eds) In Situ Hybridization Methods. Neuromethods, vol 99 (2015). Humana Press, New York, NY). Another alternative is applicable to branched-DNA FISH or to InSituPlex™ (Ultivue) imaging. It is also noted that the cross-linking approaches described herein, including, but not limited to CNVK-mediated cross-linking, can be applied to these and other approaches to cross-link labeled probes to single or repeated nucleic acid sequences associated with a target molecule or with a target-binding ligand molecule. In such approaches, cross-linking of probe as described herein (e.g., using CNVK or other cross-linking agent as described herein conjugated with the probe) to nucleic acid associated with a molecule that hybridizes or binds to a given target RNA species, e.g., in RNAScope™ or branched-DNA FISH can be used to maintain the association of the probe with the nucleic acid associated with that molecule.

In addition to the concatemer-generating methods described herein above and demonstrated in the Examples herein, other methods of repeat generation can also be used to generate a plurality of probe-binding sites on a target molecule. Rolling Circle Amplification is a non-limiting example of such an additional method for the generation of multiple probe-binding sites on a target-binding ligand (see, e.g., Mohsen, M. G. et al. The Discovery of Rolling Circle Amplification and Rolling Circle Transcription. Acc. Chem. Res. 49: 2540-2550 (2016)). Such an approach can also be combined with cross-lining as described herein, including, but not limited to CNVK-mediated cross-linking, to attach probe molecules to repeated sequences in a manner that is stable to further processing steps that might otherwise dissociate the nucleic acid probe. Where it provides an advantage, the CNVK-mediated cross-linking can also be reversed (e.g., irradiation with a separate wavelength).

Signal amplification by exchange reaction (SABER; see, e.g., Treangen T. J. et al. Repetitive DNA and next-generation sequencing: computational challenges and solutions. Nat Rev Genet. 13: 36-46 (2011)) can also be adapted in a similar manner through use of the CNVK crosslinking chemistry. For example, long repeated sequences generated per the SABER approach can be hybridized and CNVK cross-linked to an oligonucleotide on a target-binding ligand (e.g., an antibody or other target-binding ligand), and nucleic acid probe molecules can be hybridized to and similarly CNVK cross-linked to those repeated sequences to provide signal amplification (e.g., in a manner in which the repeated sequences and probes do not dissociate from the target in subsequent steps). The same approaches can be applied where repeated sequences are generated, e.g., via hybridization chain reaction (HCR; See, e.g., Hybridization chain reaction: a versatile molecular tool for biosensing, bioimaging, and biomedicine. Chem. Soc. Rev. 46: 4281-4298 (2017).

In a further modification, the concatemer approach as described herein above and demonstrated in the Examples can be adapted to use click chemistry, instead of CNVK crosslinking, to covalently bind, e.g., a repeat-containing nucleic acid to a target-binding ligand and/or to covalently bind nucleic acid probe molecules to repeats on a target-binding ligand to amplify signal; see, e.g., ClampFISH (Tavakoli, S. et al. Chapter Twenty: Click-chemistry-based amplification and detection of endogenous RNA and DNA molecules in situ using clampFISH probes. 641: 459-476 (2020)).

### Kits

In various embodiments, a kit for performing one or more of the methods described herein is provided. Such kit can include, for example, a first oligonucleotide primer, or a set of first oligonucleotide primers as described herein, and a single-stranded extender template, or a set of single-stranded extender templates suitable for performing a concatemer-generating method and/or an ordered extension or nucleic acid molecule/sequence assembly as described herein. Kits can include packaging materials for the various components and, e.g., instructions for use.

Kits can include, e.g., orthogonal sets of first oligonucleotide primers and single-stranded extender templates permitting use in multiplex. Such orthogonal sets can be optimized to avoid primer/extender cross-talk, primer-dimer formation and off-target hybridization in multiplex labeling and/or detection reactions. Kits can also include one or more target-binding ligand molecules and/or reagents for conjugating a first oligonucleotide to a target-binding ligand. Kits can also include a polymerase, e.g., a thermostable polymerase as described herein or as known in the art, as well as nucleotides and reaction/buffer components suitable for primer extension using the given polymerase enzyme. Kits can also further include one or more labeled nucleic acid probe molecules and/or reagents for labeling a nucleic acid probe molecule. The probe molecules can be complementary to, e.g., a concatemer repeat element, or to one or more elements in an ordered extension product generated as described herein. Labels can include any label described herein or known in the art, including, but not limited to fluorescent labels, metal ion labels, etc.

It should be understood that this disclosure is not limited to the particular methodology, protocols, and reagents, etc., provided herein and as such may vary. The terminology used herein is for the purpose of describing particular embodiments only, and is not intended to limit the scope of the present disclosure, which is defined solely by the claims. The invention is further illustrated by the following example, which should not be construed as further limiting.

The technology may be as described in any one of the following numbered Embodiments:

Embodiment 1: A method of labeling a target-binding ligand, the method comprising: (a) providing a conjugate of the target-binding ligand and a first oligonucleotide primer; (b) in a reaction mixture, contacting the conjugated first oligonucleotide primer with a first single-stranded extender template and a nucleic acid polymerase enzyme under conditions permitting hybridization and extension of the first oligonucleotide primer using the first single-stranded extender template; (c) heating the reaction mixture of step (b) to separate the first single-stranded extender template from extended first oligonucleotide primer produced in step (b); (d) repeating steps (b) and (c) at least once, thereby generating a concatemer comprising repeats of oligonucleotide primer sequence, conjugated to the target-binding ligand.

Embodiment 2: The method of Embodiment 1, wherein the first single-stranded extender template comprises a concatemer of at least two head-to-tail copies of a sequence complementary to the first oligonucleotide primer, and a 3′ chain terminator, such that the extender template is not extended by the polymerase.

Embodiment 3: The method of Embodiment 1 or Embodiment 2, further comprising, after at least one repeat of steps (b)-(d), the steps of: (i) adding a second oligonucleotide primer and a second single-stranded extender template oligonucleotide, wherein the second oligonucleotide primer comprises, in 5′ to 3′ order, sequence complementary to the first oligonucleotide primer, and sequence complementary to the second single-stranded extender template, wherein the second single-stranded extender template comprises a 3′ chain terminator; (ii) heating the reaction mixture to separate nucleic acid strands and cooling to permit hybridization of the second oligonucleotide primer to concatemer generated in steps (b)-(d) and hybridization of the second single-stranded extender template to the second oligonucleotide primer; (iii) extending the second oligonucleotide primer using the second single-stranded extended template oligonucleotide as template; and (iv) repeating steps (ii) and (iii) at least once, thereby generating a concatemer comprising repeats of the second oligonucleotide primer.

Embodiment 4: The method of Embodiment 1 or Embodiment 2, further comprising, after step (d): (i) adding, under conditions permitting hybridization, a nucleic acid comprising: (1) a second concatemer comprised of repeats of a second oligonucleotide sequence; and (2), a sequence complementary to the first oligonucleotide primer, such that a plurality of molecules of the nucleic acid comprising the second concatemer hybridizes via the sequence complementary to the first oligonucleotide primer to the concatemer formed in step (d).

Embodiment 5: The method of any one of Embodiments 1-4, wherein the method is performed in situ on a cell or tissue sample comprising or being assayed for target ligand.

Embodiment 6: The method of Embodiment 5, wherein the cell or tissue sample is fixed.

Embodiment 7: The method of Embodiment 5 or Embodiment 6, wherein the tissue sample is paraffin embedded.

Embodiment 8: The method of any one of Embodiments 1-7, further comprising contacting a concatemer generated in prior steps with a labeled nucleic acid probe.

Embodiment 9: The method of Embodiment 8, wherein the nucleic acid probe comprises sequence complementary to a concatemer repeat.

Embodiment 10: The method of Embodiment 8 or Embodiment 9, wherein the nucleic acid probe comprises sequence complementary to the first oligonucleotide primer.

Embodiment 11: The method of any one of Embodiments 3, or 5-7, further comprising contacting a concatemer comprised of repeats of the second oligonucleotide primer with a labeled nucleic acid probe, wherein the nucleic acid probe comprises sequence complementary to the second oligonucleotide primer.

Embodiment 12: The method of any one of Embodiments 4, or 5-7, further comprising contacting a concatemer comprised of repeats of the second oligonucleotide sequence with a labeled nucleic acid probe, wherein the nucleic acid probe comprises sequence complementary to the second oligonucleotide sequence.

Embodiment 13: The method of any one of Embodiments 8-12, wherein a plurality of labeled nucleic acid probe molecules hybridizes to a plurality of concatemeric repeats.

Embodiment 14: The method of any one of Embodiments 1-8, wherein the target-binding ligand comprises an antibody or antigen-binding fragment thereof.

Embodiment 15: The method of any one of Embodiments 1-14, wherein a plurality of different target-binding ligands are labeled in multiplex via different orthogonal oligonucleotide primer/single-stranded extender template pairs.

Embodiment 16: A method of detecting a target molecule in situ in a preparation of cells or tissue, the method comprising: (a) contacting the preparation of cells or tissue with a target-binding ligand conjugated to a first oligonucleotide primer, under conditions permitting specific binding of the target-binding ligand to the target molecule; (b) adding to the preparation of cells or tissue a reaction mixture comprising a first single-stranded extender template and a nucleic acid polymerase enzyme under conditions permitting hybridization and extension of the first oligonucleotide primer using the first single-stranded extender template; (c) heating the reaction mixture of step (b) to separate the first single-stranded extender template from extended first oligonucleotide primer produced in step (b); (d) repeating steps (b) and (c) at least once, thereby generating a concatemer comprising repeats of the first oligonucleotide primer sequence, conjugated to the target-binding ligand; (e) contacting the concatemer generated in step (d) with a labeled nucleic acid probe; and (f) detecting labeled nucleic acid probe, wherein detection of labeled nucleic acid probe indicates the presence and location of the target molecule in the preparation of cells or tissue.

Embodiment 17: The method of Embodiment 16, wherein the first single-stranded extender template comprises a concatemer of at least two head-to-tail copies of a sequence complementary to the first oligonucleotide primer, and a 3′ chain terminator, such that the extender template is not extended by the polymerase.

Embodiment 18: The method of Embodiment 16 or Embodiment 17, wherein the target-binding ligand comprises an antibody or antigen-binding fragment thereof.

Embodiment 19: The method of any one of Embodiments 16-18, wherein the labeled nucleic acid probe comprises sequence complementary to the first oligonucleotide primer.

Embodiment 20: The method of any one of Embodiments 16-19, wherein the labeled nucleic acid probe comprises sequence complementary to a concatemer repeat.

Embodiment 21: The method of any one of Embodiments 16-20, wherein a plurality of labeled nucleic acid probe molecules hybridizes to a plurality of concatemer repeats conjugated to the target-binding ligand.

Embodiment 22: The method of any one of claims 16-18, further comprising the steps, after step (d), and before step (e), of: (i) adding a second oligonucleotide primer and a second single-stranded extender template oligonucleotide, wherein the second oligonucleotide primer comprises, in 5′ to 3′ order, sequence complementary to the first oligonucleotide primer, and sequence complementary to the second single-stranded extender template, wherein the second single-stranded extender template comprises a 3′ chain terminator; (ii) heating the reaction mixture to separate nucleic acid strands and cooling to permit hybridization of the second oligonucleotide primer to concatemer generated in step (d) and hybridization of the second single-stranded extender template to the second oligonucleotide primer; (iii) extending the second oligonucleotide primer using the second single-stranded extended template oligonucleotide as template; and (iv) repeating steps (ii) and (iii) at least once, thereby generating a concatemer comprising repeats of the second oligonucleotide primer.

Embodiment 23: The method of Embodiment 22, wherein the labeled nucleic acid probe comprises sequence complementary to the second oligonucleotide primer.

Embodiment 24: The method of Embodiment 22 or 23, wherein a plurality of labeled nucleic acid probe molecules hybridizes to a plurality of concatemer repeats conjugated to the target-binding ligand.

Embodiment 25: The method of any one of Embodiments 16-18, further comprising, after step (d) and before step (e): (i) adding, under conditions permitting hybridization, a nucleic acid comprising: (1) a second concatemer comprised of repeats of a second oligonucleotide sequence; and (2), a sequence complementary to the first oligonucleotide primer, such that a plurality of molecules of the nucleic acid comprising the second concatemer hybridizes via the sequence complementary to the first oligonucleotide primer to the concatemer comprising repeats of the first oligonucleotide primer sequence formed in step (d).

Embodiment 26: The method of Embodiment 25, wherein the labeled nucleic acid probe comprises sequence complementary to the second oligonucleotide sequence.

Embodiment 27: The method of Embodiment 25 or 26, wherein a plurality of labeled nucleic acid probe molecules hybridizes to a plurality of second concatemer repeats.

Embodiment 28: The method of any one of Embodiments 16-27, wherein the preparation of cells or tissue is fixed.

Embodiment 29: The method of Embodiment 28, wherein the preparation of tissue is paraffin embedded.

Embodiment 30: A method of labeling a set of target-binding ligands, the method comprising: (a) providing a set of orthogonal first oligonucleotide primer and first single-stranded extender template oligonucleotide pairs, wherein the first oligonucleotide primer of each pair has a different sequence from other first oligonucleotide primers in the set, and is conjugated to a different member of a set of target-binding ligands; (b) in a reaction mixture, contacting the conjugated first oligonucleotide primers with the first single-stranded extender templates of the set of orthogonal pairs and a nucleic acid polymerase under conditions permitting hybridization and extension of the first oligonucleotide primers using the first single-stranded extender templates of the set of orthogonal pairs; (c) heating the reaction mixture of step (b) to separate the first single-stranded extender templates from extended first oligonucleotide primers produced in step (b); (d) repeating steps (b) and (c) at least once, thereby generating, on each member of the set of target ligands, a concatemer comprising repeats of oligonucleotide primer sequence, conjugated to the target-binding ligand.

Embodiment 31: The method of Embodiment 30, wherein the first single-stranded extender template in each orthogonal first oligonucleotide primer/first single-stranded extender pair comprises a concatemer of at least two head-to-tail copies of a sequence complementary to the first oligonucleotide primer, and a 3′ chain terminator, such that the extender template is not extended by the polymerase.

Embodiment 32: The method of Embodiment 30 or Embodiment 31, further comprising, after step (d), the steps of: (i) adding a second set of orthogonal second oligonucleotide primer, second single-stranded extender template oligonucleotide pairs, wherein the second oligonucleotide primer in each pair comprises, in 5′ to 3′ order, sequence complementary to a member of the set of first oligonucleotide primers, and sequence complementary to the second single-stranded extender template of the second set of orthogonal oligonucleotide pairs, wherein each second single-stranded extender template comprises a 3′ chain terminator; (ii) heating the reaction mixture to separate nucleic acid strands and cooling to permit hybridization of the second oligonucleotide primers to concatemers generated in steps (b)-(d) and hybridization of the second single-stranded extender templates to the second oligonucleotide primers; (iii) extending the second oligonucleotide primers using the second single-stranded extended template oligonucleotides as template; and (iv) repeating steps (ii) and (iii) at least once, thereby generating, on respective members of the set of target-binding ligands, a concatemer comprising repeats of a respective second oligonucleotide primer.

Embodiment 33: The method of Embodiment 30 or 31, further comprising contacting concatemers generated in prior steps with a plurality of labeled nucleic acid probes, wherein each different probe is labeled with a distinguishable detectable label moiety, and wherein members of the plurality of labeled nucleic acid probes comprise sequence complementary to respective members of the set of first oligonucleotide primers.

Embodiment 34: The method of Embodiment 32, further comprising contacting concatemers generated in step (iv) with a plurality of labeled nucleic acid probes, wherein each different probe is labeled with a distinguishable detectable label moiety, and wherein members of the plurality of labeled nucleic acid probes comprise sequence complementary to respective members of the set of second oligonucleotide primers.

Embodiment 35: The method of Embodiment 33 or 34, wherein, for respective members of the set of target-binding ligands, a plurality of labeled nucleic acid probe molecules hybridizes to a plurality of concatemeric repeats.

Embodiment 36: The method of any one of Embodiments 30-35, wherein the method is performed in situ on a cell or tissue sample comprising or being assayed for target ligand.

Embodiment 37: The method of Embodiment 36, wherein the preparation of cells or tissue is fixed.

Embodiment 38: The method of Embodiment 37, wherein the preparation of tissue is paraffin embedded.

Embodiment 39: The method of any one of Embodiments 30-38, wherein the target-binding ligand comprises an antibody or antigen-binding fragment thereof.

Embodiment 40: A method of detecting a set of target molecules in situ in a preparation of cells or tissue, the method comprising: (a) providing a set of target-binding ligand molecules, wherein members of the set of target-binding ligand molecules are conjugated to respective first oligonucleotide primer members of a set of orthogonal first oligonucleotide primer and first single-stranded extender template oligonucleotide pairs, wherein the first oligonucleotide primer of each pair has a different sequence from other first oligonucleotide primers in the set, and is conjugated to a different member of the set of target-binding ligand molecules; (b) contacting the preparation of cells or tissue with the set of target-binding ligand molecules under conditions permitting specific binding of the target-binding ligand molecules to target molecules present in the preparation of cells or tissue; (c) adding to the preparation of cells or tissue a reaction mixture comprising first single-stranded extender templates of the set of orthogonal first oligonucleotide primer and first single-stranded extender template oligonucleotide pairs, and a nucleic acid polymerase, under conditions permitting hybridization and extension of the first oligonucleotide primers using the respective first single-stranded extender templates; (d) heating the reaction mixture of step (c) to separate the first single-stranded extender templates from extended first oligonucleotide primers produced in step (c); (e) repeating steps (c) and (d) at least once, thereby generating, on respective members of the set of target-binding ligand molecules, a concatemer comprising repeats of the respective first oligonucleotide primer sequence, conjugated to the target-binding ligand molecule; (f) contacting the concatemers generated in step (e) with a plurality of distinguishably-labeled nucleic acid probes; and (g) detecting labeled nucleic acid probes, wherein detection of labeled nucleic acid probes indicates the presence and location of the target molecules in the preparation of cells or tissue.

Embodiment 41: The method of Embodiment 40, wherein the first single-stranded extender template comprises a concatemer of at least two head-to-tail copies of a sequence complementary to the first oligonucleotide primer, and a 3′ chain terminator, such that the extender template is not extended by the polymerase.

Embodiment 42: The method of Embodiment 40 or Embodiment 41, further comprising, after step (e), and before step (f), the steps of: (i) adding a second set of orthogonal second oligonucleotide primer, second single-stranded extender template oligonucleotide pairs, wherein the second oligonucleotide primer in each pair comprises, in 5′ to 3′ order, sequence complementary to a member of the set of first oligonucleotide primers, and sequence complementary to the second single-stranded extender template of the second set of orthogonal oligonucleotide pairs, wherein each second single-stranded extender template comprises a 3′ chain terminator; (ii) heating the reaction mixture to separate nucleic acid strands and cooling to permit hybridization of the second oligonucleotide primers to concatemers generated in steps (c)-(e) and hybridization of the second single-stranded extender templates to the second oligonucleotide primers; (iii) extending the second oligonucleotide primers using the second single-stranded extended template oligonucleotides as template; and (iv) repeating steps (ii) and (iii) at least once, thereby generating, on respective members of the set of target-binding ligands, a concatemer comprising repeats of a respective second oligonucleotide primer.

Embodiment 43: The method of any one of Embodiments 40-42, wherein the plurality of distinguishably-labeled nucleic acid probes comprises sequences complementary to the respective first oligonucleotide primer members in the set of orthogonal first oligonucleotide primer and first single-stranded extender template oligonucleotide pairs.

Embodiment 44: The method of Embodiment 42, wherein the plurality of distinguishably-labeled nucleic acid probes comprises sequences complementary to respective second oligonucleotide primer members of the second set of orthogonal second oligonucleotide primer, second single-stranded extender template oligonucleotide pairs.

Embodiment 45: The method of any one of Embodiments 40-44, wherein the target-binding ligand comprises an antibody or antigen-binding fragment thereof.

Embodiment 46: The method of any one of Embodiments 40-45, wherein a plurality of labeled nucleic acid probe molecules hybridizes to a plurality of concatemer repeats conjugated to the target-binding ligand.

Embodiment 47: A kit comprising reagents for performing the methods of any one of Embodiments 1-46.

Embodiment 48: A target-binding ligand comprising a concatemer produced by the method of any one of Embodiments 1-15.

Embodiment 49: The target-binding ligand of Embodiment 48, wherein the target-binding ligand comprises an antibody or antigen-binding fragment thereof.

Embodiment 50: A method of generating a nucleic acid strand comprising concatemeric repeats of a given sequence, the method comprising: a) providing in a reaction mixture, a first oligonucleotide primer and a first extender template oligonucleotide comprising a concatemer of at least two head-to-tail copies of sequence complementary to the first oligonucleotide primer, wherein the first extender template comprises a chain terminator at its 3′ end; b) incubating the reaction mixture under conditions permitting hybridization of the first oligonucleotide primer to the first extender template oligonucleotide; c) extending the hybridized first primer with a nucleic acid polymerase enzyme, thereby generating an extended nucleic acid strand complementary to the first extender template; d) heating the reaction mixture to separate the extended nucleic acid strand complementary to the first extender template from the first extender template; e) cooling the heated products to permit hybridization of the first extender template oligonucleotide to the extended nucleic acid strand complementary to the first extender template generated in step (c); f) extending the extended nucleic acid strand complementary to the first extender template generated in step (c) with the polymerase, thereby generating an extended nucleic acid strand comprising concatemeric repeats of the sequence comprised by the first oligonucleotide primer.

Embodiment 51: The method of Embodiment 50, further comprising repeating steps (d) to (f) at least once.

Embodiment 52: The method of Embodiment 51, comprising repeating steps (d) to (f) at least n times, wherein each iteration of steps (d) to (f) increases the concatemeric nucleic acid length by one concatemeric repeat.

Embodiment 53: The method of any one of Embodiments 50-52, wherein step (a) comprises providing a set of orthogonal first oligonucleotide primer and first extender template oligonucleotide pairs, such that steps (b)-(f) generate a multiplex set of concatemers, each comprising concatemeric repeats of the respective orthogonal oligonucleotide primer sequences.

Embodiment 54: The method of any one of Embodiments 51-53, further comprising, after one or more repetitions of steps (d) to (f): i) adding a second oligonucleotide primer and a second extender template oligonucleotide to the reaction, wherein the second oligonucleotide primer comprises a 5′ proximal sequence complementary to the first oligonucleotide primer sequence and a 3′ proximal sequence complementary to one of at least two repeat portions of the second extender template oligonucleotide, wherein the second extender template oligonucleotide comprises at least two head-to-tail repeats of sequence complementary to the second oligonucleotide primer and a chain terminator at its 3′ end; ii) hybridizing the second oligonucleotide primer to one or more concatemeric repeats on a concatemer formed after one or more repetitions of steps (d) to (f); iii) hybridizing the second extender template oligonucleotide to the second oligonucleotide primer; iv) extending the second oligonucleotide primer with the polymerase enzyme, thereby generating an extended nucleic acid strand complementary to the second extender template; v) heating the reaction mixture to separate the extended nucleic acid strand complementary to the second extender template from the second extender template; vi) cooling the heated products to permit hybridization of the second extender template oligonucleotide to the extended nucleic acid strand complementary to the second extender template generated in step (iv); vii) extending the extended nucleic acid strand complementary to the second extender template generated in step (iv) with the polymerase, thereby generating an extended nucleic acid strand comprising concatemeric repeats of the sequence comprised by the second oligonucleotide primer.

Embodiment 55: The method of Embodiment 54, further comprising repeating steps (v) to (vii) at least once.

Embodiment 56: The method of Embodiment 54 or 55, wherein step (i) comprises providing a set of orthogonal second oligonucleotide primer and second extender template oligonucleotide pairs, such that steps (ii)-(vii) generate a multiplex set of concatemers, each comprising concatemeric repeats of the respective orthogonal oligonucleotide primer sequences.

Embodiment 57: The method of any one of Embodiments 50-56, wherein the first oligonucleotide primer is conjugated to a target-binding ligand via its 5′ end.

Embodiment 58: The method of Embodiment 57, wherein the target-binding ligand is a polypeptide.

Embodiment 59: The method of Embodiment 58, wherein the polypeptide comprises an antibody or antigen-binding fragment thereof.

Embodiment 60: The method of any one of Embodiments 57-59, further comprising contacting the target-binding ligand with a cell or tissue preparation comprising or being assayed for the presence of the target.

Embodiment 61: The method of Embodiment 60, wherein the contacting is performed before concatemer-generating steps (a)-(f) or (i)-(vii).

Embodiment 62. The method of any one of Embodiments 50-61, further comprising contacting an extended nucleic acid strand comprising concatemeric repeats generated according to steps (a)-(f) and/or (i)-(vii) with a nucleic acid probe comprising the complement of a concatemeric repeat sequence, and a detectable label.

Embodiment 63: The method of any one of Embodiments 50-53, wherein the method generates linear concatemers comprising the first oligonucleotide primer sequence.

Embodiment 64: The method of Embodiment 54, wherein the method generates branched concatemers comprising concatemers of the first oligonucleotide primer sequence, complexed with concatemers of the second oligonucleotide primer sequence.

Embodiment 65: The method of any one of Embodiments 50-64, wherein the method is performed in contact with a cell or tissue preparation.

Embodiment 66: The method of Embodiment 65, wherein the cell or tissue preparation is fixed.

Embodiment 67: A method of labeling a target-binding ligand, the method comprising: performing the method of any one of Embodiments 50-65, wherein the first oligonucleotide primer is conjugated to the target-binding ligand.

Embodiment 68: The method of Embodiment 67, wherein the target-binding ligand comprises an antibody or antigen-binding fragment thereof.

Embodiment 69: The method of Embodiment 67 or 68, wherein the first oligonucleotide primer is conjugated to the target binding ligand in a manner that permits extension of the primer from its 3′ end.

Embodiment 70: The method of any one of Embodiments 67-69, wherein the method is performed in contact with a cell or tissue preparation.

Embodiment 71: The method of Embodiment 65, wherein the cell or tissue preparation is fixed.

Embodiment 72: The method of any one of Embodiments 67-71, further comprising contacting the concatemer generated in Embodiment 50 or 51 with a labeled nucleic acid probe, and detecting labeled nucleic acid probe associated with concatemer, wherein detection of labeled nucleic acid probe indicates the presence and location of the target molecule.

Embodiment 73: The method of any one of Embodiments 1-72, wherein the nucleic acid polymerase enzyme is a thermostable nucleic acid polymerase enzyme.

Embodiment 74: A method of generating a nucleic acid molecule, the method comprising: (a) providing a first oligonucleotide primer; (b) in a reaction mixture, contacting the first oligonucleotide primer with a first single-stranded extender template and a nucleic acid polymerase enzyme under conditions permitting hybridization and extension of the first oligonucleotide primer using the first single-stranded extender template, wherein the first single-stranded extender template comprises, in a 5′ to 3′ direction, a first extension template element, a sequence complementary to the first oligonucleotide primer and a 3′ chain terminator, such that the extender template is not extended by the polymerase; (c) heating the reaction mixture of step (b) to separate the first single-stranded extender template from extended first oligonucleotide primer produced in step (b); (d) contacting the extended first oligonucleotide primer with a second single-stranded extender template under conditions permitting hybridization and extension of the first extended oligonucleotide primer generated in step (b) using the second single-stranded extender template, wherein the second single-stranded extender template comprises, in a 5′ to 3′ direction, a second extension template element, a copy of the first extension template element, and a 3′ chain terminator; wherein steps (a)-(d) generate a nucleic acid molecule comprising, in 5′ to 3′ order, the first oligonucleotide primer sequence, the complement of the first extension template element, and the complement of the second extension template element.

Embodiment 75: The method of Embodiment 74, wherein the polymerase enzyme is a thermostable polymerase enzyme.

Embodiment 76: The method of Embodiment 74 or Embodiment 75, further including repeating steps (b) and (c) at least once before step (d), wherein each repeat of steps (b) and (c) extends the generated nucleic acid molecule by one copy of the complement of the first extension template element.

Embodiment 77: The method of any one of Embodiments 74-76, further including, at least one iteration of (e) heating the reaction mixture of step (d) to separate the second single-stranded extender template from extended first oligonucleotide primer produced in step (d); and (f) repeating step (d), wherein each iteration of steps (e) and (f) extends the generated nucleic acid molecule by one copy of the complement of the second extension element.

Embodiment 78. The method of any one of Embodiments 74-77, further comprising heating the reaction mixture after step (d) and contacting the nucleic acid molecule generated in steps (a) to (d) with at least one additional single-stranded extender template under conditions permitting hybridization and extension of the generated nucleic acid molecule, wherein the additional single-stranded extender template comprises, in a 5′ to 3′ direction, an additional extension template element, a copy of the extension template element from the previous round of extension, and a 3′ chain terminator, wherein the generated nucleic acid molecule is extended to include the complement of the additional extension template element.

Embodiment 79: The method of any one of Embodiments 74-78, wherein the first oligonucleotide is conjugated to a target-binding ligand.

Embodiment 80: The method of any one of Embodiments 74-79 which is performed in contact with a cell or tissue sample.

Embodiment 81: The method of any one of Embodiment 74-80, further comprising contacting the generated nucleic acid molecule with a labeled nucleic acid probe comprising sequence of one or more of the extension template elements.

Embodiment 82: A kit for performing one or more of the methods as described herein, the kit comprising: a) a first oligonucleotide primer or a set of first oligonucleotide primers; b) a single-stranded extender template or a set of single-stranded extender templates; and c) an order extension or nucleic acid molecule.

Embodiment 83: The kit of Embodiment 82, wherein the first oligonucleotide primer or the set of first oligonucleotide primers and single stranded extender template or the set of single stranded extender templates are orthogonal sets.

Embodiment 84: The kit of Embodiment 83, wherein the orthogonal sets are optimized to avoid primer/extender cross-talk, primer-dimer formation, and off-target hybridization.

Embodiment 85: The kit of Embodiment 82, wherein the single-stranded extender template or the set of single-stranded extender templates are suitable for performing a concatemer-generated method.

Embodiment 86: The kit of Embodiment 82, further comprising packaging materials for the various components and instructions for use.

Embodiment 87: The kit of Embodiment 82, further comprising one or more target-binding ligand molecules or reagents for conjugating a first oligonucleotide to a target-binding ligand.

Embodiment 88: The kit of Embodiment 82, further comprising a thermostable polymerase, nucleotides, reaction buffer components, and reagents for labeling a nucleic acid probe molecule.

Embodiment 89: The kit of Embodiment 88, further comprising a probe molecule complementary to a concatemeric repeat element.

Embodiment 90: The kit of Embodiment 88, wherein the probe molecule can be complementary to one or more elements in an ordered extension product generated.

Specific elements of any of the disclosed embodiments can be combined or substituted for elements in other embodiments. Furthermore, while advantages associated with certain embodiments of the disclosure have been described in the context of these embodiments, other embodiments may also exhibit such advantages, and not all embodiments need necessarily exhibit such advantages to fall within the scope of the disclosure.

### EXAMPLES

The following examples illustrate some embodiments and aspects of the invention. It will be apparent to those skilled in the relevant art that various modifications, additions, substitutions, and the like can be performed without altering the spirit or scope of the invention, and such modifications and variations are encompassed within the scope of the invention as defined in the claims which follow. The following examples do not in any way limit the invention.

### Example 1: Method for Highly Multiplexed, Thermal Controllable DNA Extension and Its Applications

TCE (thermal controllable DNA extension) technology (FIG. 1A-1C) achieves highly multiplexed, uniform, and controllable DNA amplification through high and low temperature cycling and extension mediated by non-extendable single-stranded extenders (ssEXTers) and DNA polymerases.

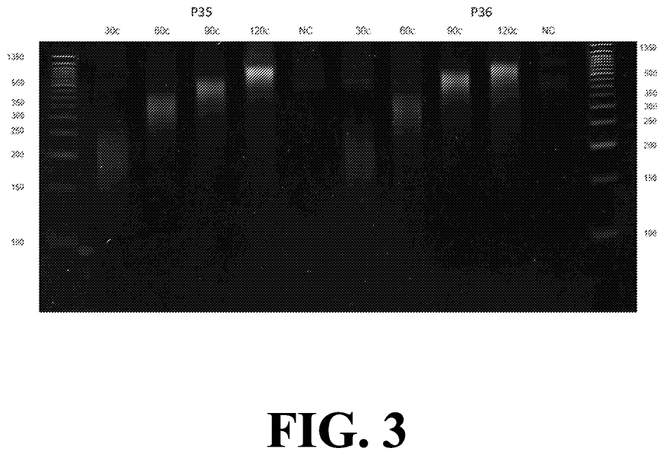

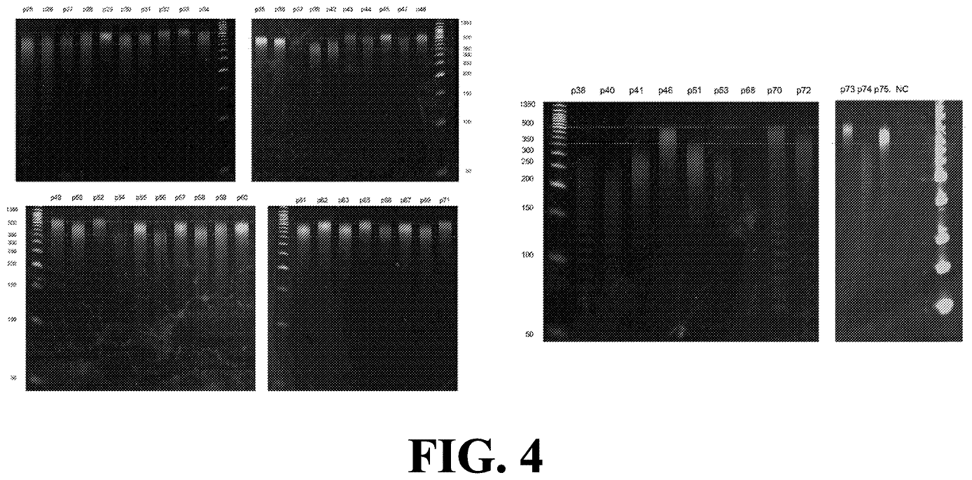

TCE can be used to generate concatemers with repeated sequences (FIG. 1A). For example, if one wishes to generate concatemers with multiple a repeats, one can mix primer a with ssEXTers containing two repeats of a* (reverse complement of a), DNA polymerases, buffer, and dNTPs. The reaction starts at a low temperature so that primer a will bind to the the a*a* ssEXTer. DNA polymerase will extend the primer a to form an aa sequence bound by the ssEXTer. The temperature can be raised so that the aa sequence will be melted from the ssEXTer. Once the temperature is lowered, the aa sequence can bind to the ssEXTer again and get extended by the DNA polymerase to form a aaa sequence. The ssEXTers has a stopper on the 3′ end so they are not extendable. The temperature cycling can be performed multiple times to control the length of extension on the primers. Different cycles of amplification were tested for two 9 nt primers (FIG. 3). The results showed that the amplification is tightly controlled by the number of thermal cycles. Since the extension is an on-and-off reaction controlled by the thermal cycling, the amplification should be high uniform for different sequences. 50 different 9 nt sequences were tested for their amplification uniformity (FIG. 4). Little sequence dependence was observed for the extension efficiency.

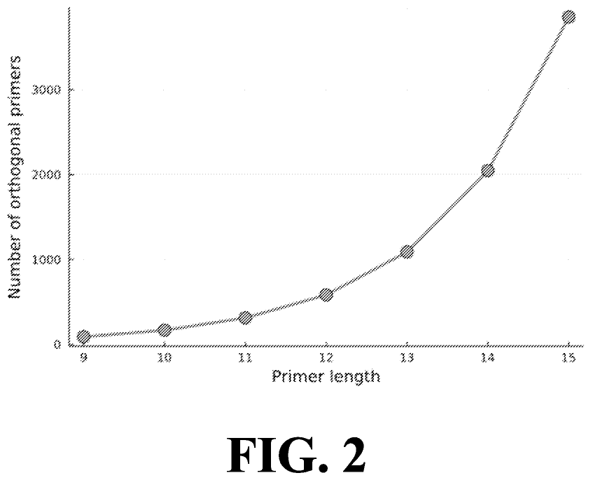

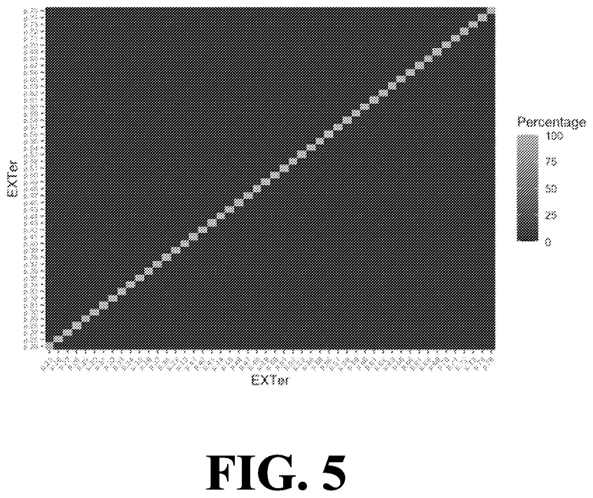

If multiple orthogonal sequences are designed a to n, TCE can be used to achieve multiplexed concatemer generation (FIG. 1B). A low temperature can be set which all the single repeat a to n will bind to their designated ssEXTers. A high temperature can be set which all the double repeats aa to nn will dissociate from their designated ssEXTers. If the sequences are a to n and their ssEXTers a*a* to n*n* in the same reaction, thermal cycling can be performed between these two temperatures to achieve multiplexed amplification. Since the sequences are orthogonal to each other, there will be little crosstalk extension. The total number of multiplexity correspond to the length of the single repeat sequences (FIG. 2). With 15 nt single repeat length, amplification can be achieved of 3855 different sequences. To verify the hypothesis, 50 orthogonal sequences were designed with 9 nt in length. The 50 plex was performed extension in a test tube and performed next generation sequencing to sequence the generated concatemers (FIG. 5). The results showed 45 ssEXTers have >99.5% specificity and 48 ssEXTers have >99% specificity.

TCE can also be used for primer exchange reactions (FIG. 1C) to generate concatemers comprising or consisting of different sequences. For example, to generate concatemers containing orthogonal sequences a1 to an, multiple ssEXTers can be designed: a1* a2*, a2* a3*, . . . , and an-1* an* and extend primer a1 with these ssEXTers by TCE. After multiple cycles of extension, concatemers should be able to be obtained containing sequence a1 to an.

TCE chemistry has multiple application scenarios including but not limited to imaging (see scenario 1), mass cytometry (see scenario 2), flow cytometry (see scenario 3), and immunoblotting (see scenario 4).

### Example 2: Scenario 1: Highly Multiplexing Signal Amplified Multi-Omics Imaging Via In-Situ Thermal-Cyclic DNA Extension

ISE-imaging (in-situ extension imaging) takes advantage of small size and high orthogonality of DNA molecules to achieve high signal to noise and high multiplex multi-omics imaging. It utilizes polymerase mediated iterative in situ DNA extension controlled by thermocycling to achieve highly multiplexed amplification.

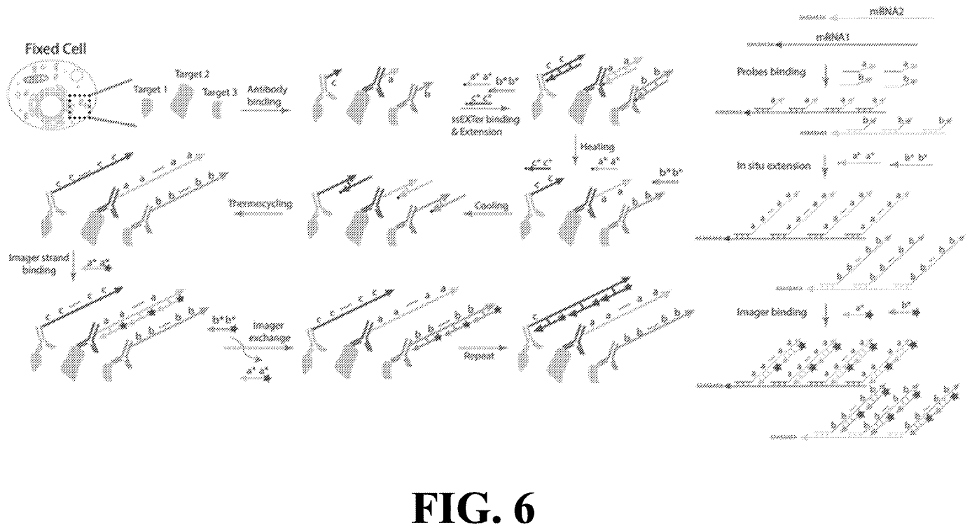

Specifically, the samples of interest are first stained by antibodies conjugated with orthogonal short initiator oligos and/or are hybridized DNA probes with orthogonal short initiator sequences to RNAs (FIG. 6). Multiple 3′ sealed single-stranded extenders (ssEXTers) containing two repeats of the initiator sequences will then be added to the samples. Each of the ssEXTers will bind and extend the specific initiators by DNA polymerization at low temperature. The samples will then be heated to high temperature to melt the ssEXTers (all the ssEXTers have melting temperature lower than 56° C. in the reaction buffer). Multiple rounds of temperature cycling will be performed to allow for ssEXTers reannealing and extending repeatedly, leading to the generation of long concatemers in situ. Orthogonal fluorophore-labeled imagers that hybridize to the concatemers can then be added for signal amplification. Subsequently, rapid imager exchange rounds can be performed to allow for high multiplexity.

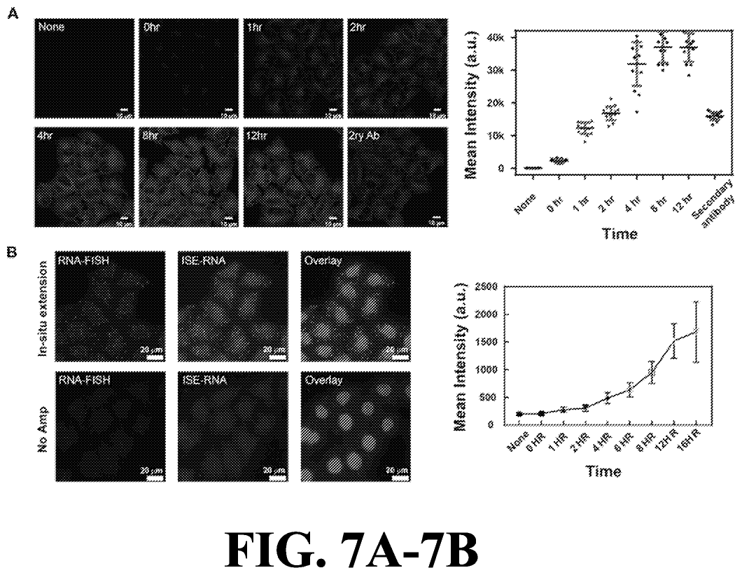

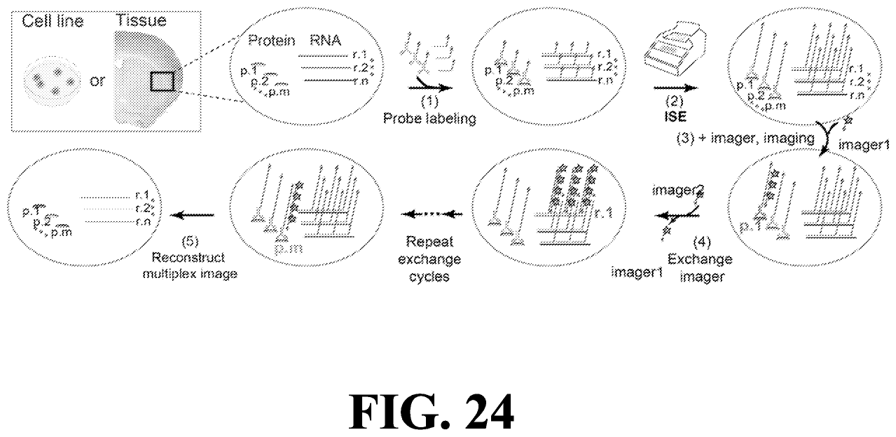

The signal amplification is highly tunable by changing the number of thermo-cycling rounds. Take β-tubulin in immortal cell line (Hela) as an example of the protein target (FIG. 7A, FIG. 24). The cell will first be fixed with paraformaldehyde. Then initiator conjugated primary antibodies will be stained to the sample. The sample will be transferred to thermo-cycler. DNA initiator on β-tubulin antibodies will be linearly extended to DNA concatemers by thermocycling reaction. Fluorophore-labeled DNA imager will be supplied and hybridize to the concatemers. Amplified signal of the target molecules will then be acquired with fluorescence microscope. AXE achieved more than 14-fold of amplification for 4 hrs of thermocycling (~130 cycles) and plateaued at 8 hrs of thermocycling (~260 cycles) for 16.5-fold amplification (FIG. 7A, FIG. 24). Similarly, the RNA target (APC gene) can be amplified in Hela cell (FIG. 7B, FIG. 24). ~8 fold amplification is achieved with 16 hrs of thermocycling.

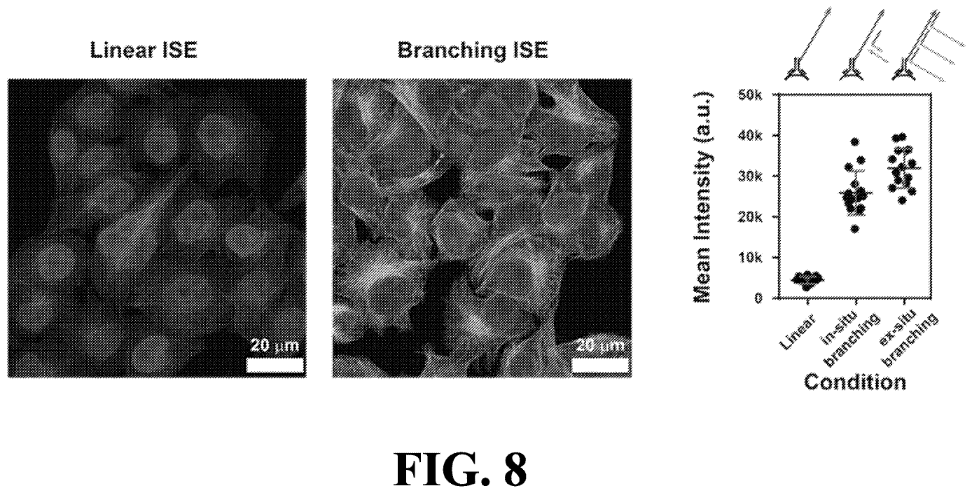

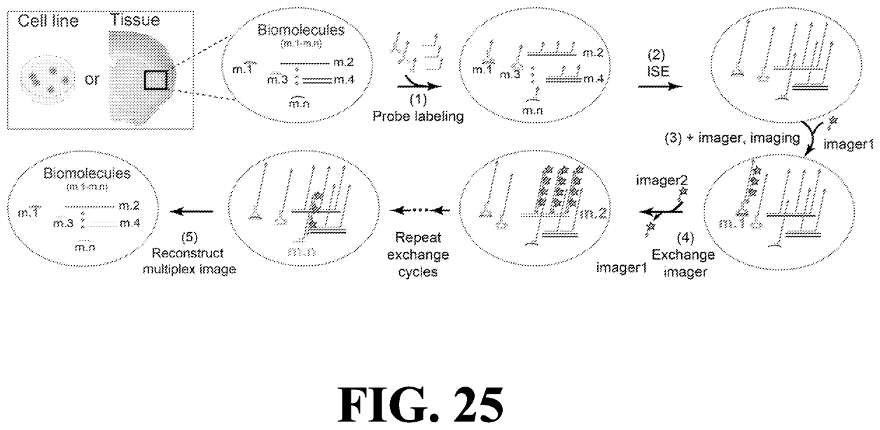

The signal amplification from ISE imaging can be further enhanced by branching the DNA concatemers. After the initial linear extension of concatemers, secondary initiator containing probes will be added to hybridize with primary linear concatemers. This will create more imager binding sites and enable additional amplification. Alternatively, the secondary long concatemer can also be generated separately in an ex-situ reaction and added to the sample to make the total amplification to >100-fold (FIG. 8, FIG. 25).

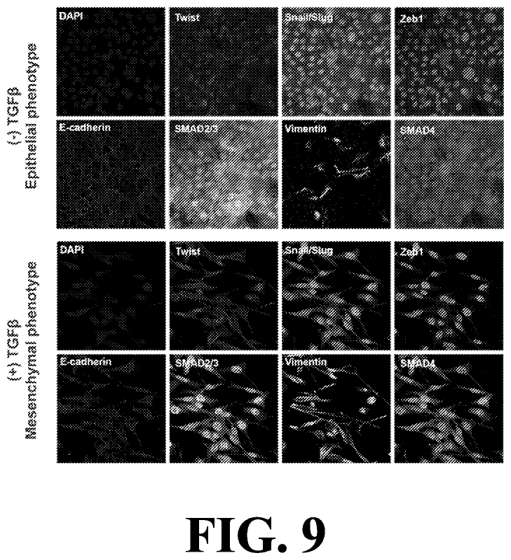

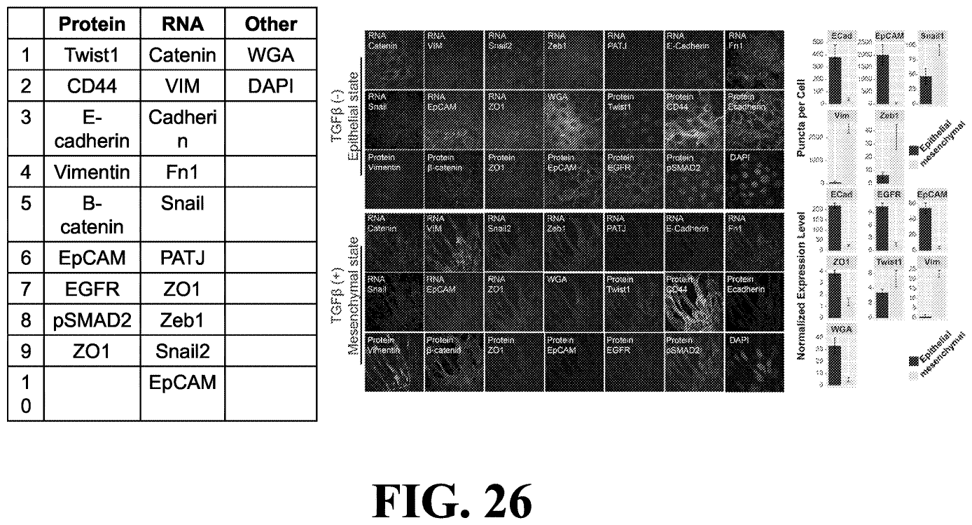

Multiplexing ISE imaging can be achieved by co-staining proteins in specimen. (FIG. 9, FIG. 26).

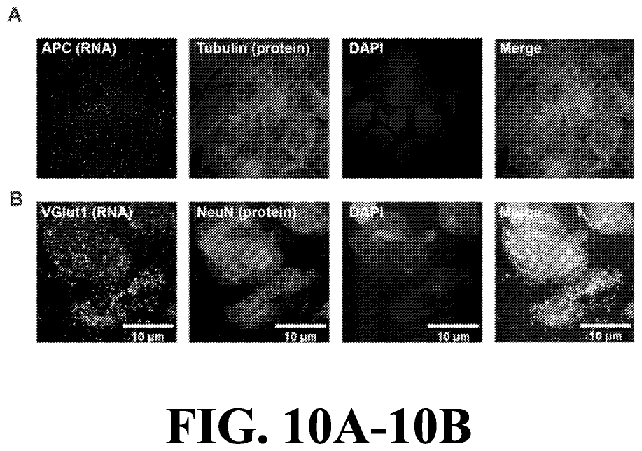

For example, ISE imaging successfully displays the expression level changes of marker proteins during the Epithelial-Mesenchymal Transition (EMT) process of Py2T model cell (FIG. 9). In addition, co-imaging of RNA and protein can be accomplished by ISE multiplex imaging. Both RNA probes hybridization and antibody staining are carried out, followed by subsequential ISE extension in the tissue (FIG. 10).

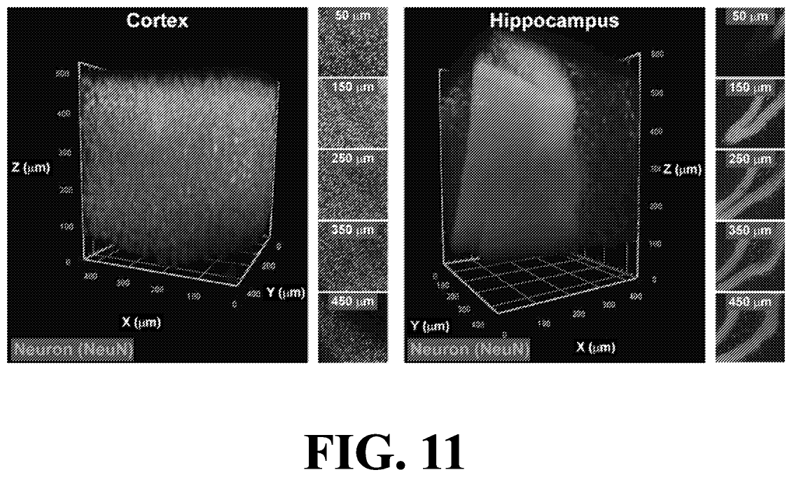

The ISE protocol utilizes short conjugation oligos and short ssEXTs for amplification, which in principle leads to high penetration of probes in thick tissue. Therefore, ISE imaging enables the cell phenotyping within thick samples. Combining with the conventional tissue clearing method (SHIELD), ISE imaging achieves labeling and signal amplification of neuronal protein marker in cortical and hippocampal regions of 0.5 mm mouse brain (FIG. 11).

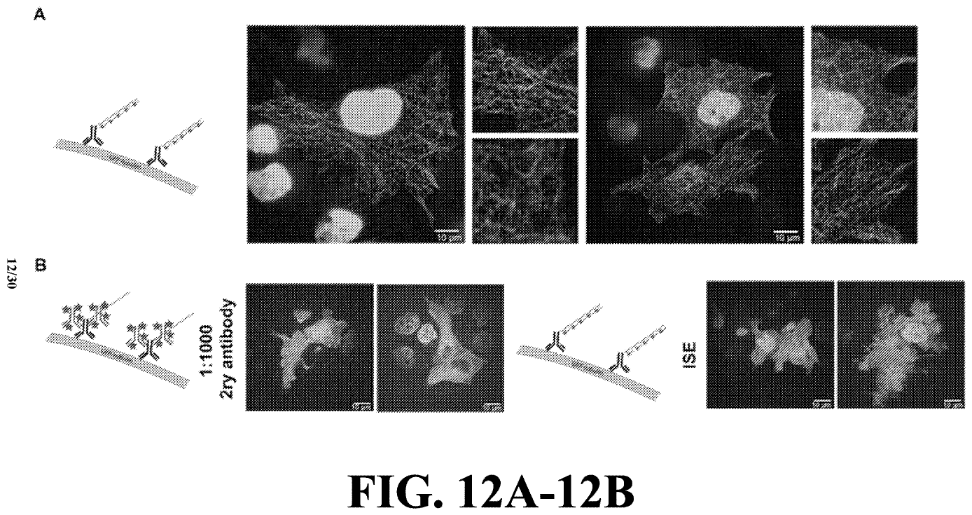

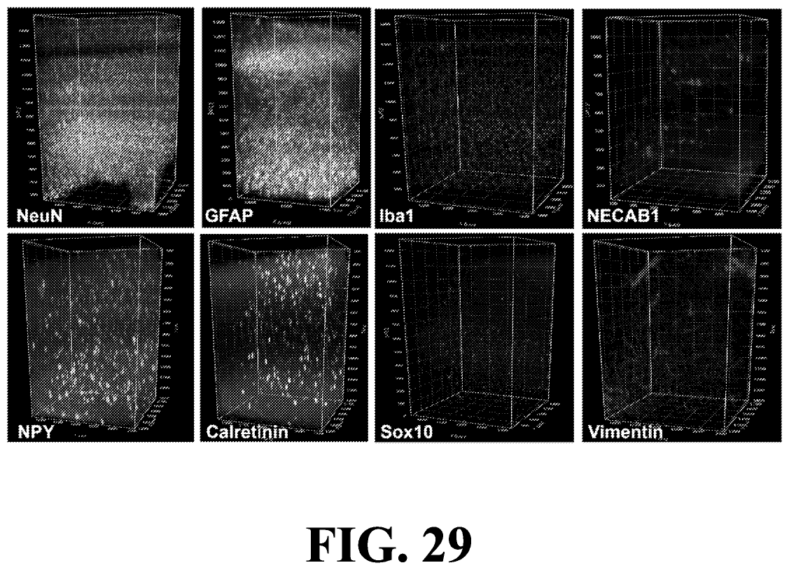

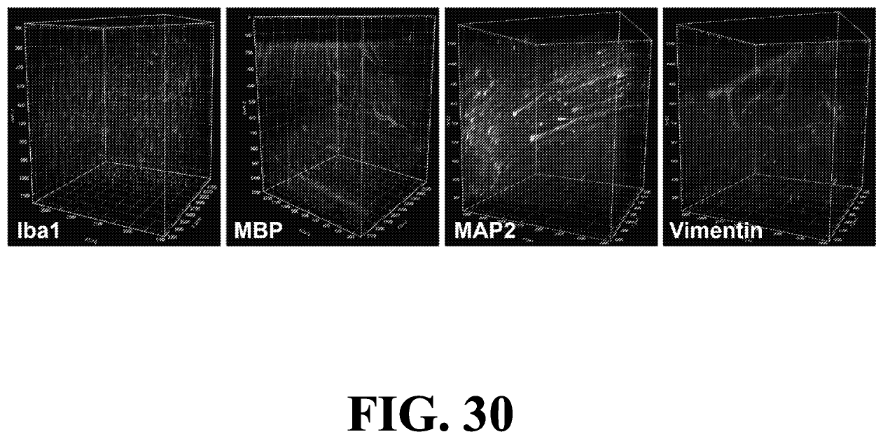

ISE amplifies signal with high signal-to-noise. Many of the previous amplification methods rely on the adding of ex-situ synthesized preamplifiers to targets of interest. Any nonspecific binding of the preamplifiers may cause unspecific signal amplification leading to a compromised signal-to-noise ratio. In the ISE protocol, no preamplifiers are added and signals will only be amplified from the initiators in situ. Moreover, for antibody conjugation, only a short 11 nt initiator is used, therefore minimizing the non-specific binding of the antibodies. To demonstrate the minimal non-specific signal from ISE protocol, Hela cells are transfected with Tubulin-GFP fusion gene. Then, the anti-GFP primary antibodies is diluted to 100 pg/mL and signal-amplified by ISE protocol. The majority of the signal puncta in the cells are observed to colocalize with high Tubulin-GFP expression region (FIG. 12A). In contrast, signal amplification by fluorophore-tagged secondary antibody leads to high non-specific background signal in the region lacking Tubulin-GFP expression (FIG. 12B). Additional imaging using ISE is shown in FIG. 29, demonstrating that ISE is compatible with various types of clearing protocols for thick tissue imaging in mice. Human can also be used (FIG. 30) in ISE protein imaging. Samples are photo-bleached by LED to reduce auto-fluorescence.

### Example 3: Scenario 2: Engineering Dynamic DNA Nano-Devices to Amply Signal in Mass Cytometry Analysis

Introduction. Mass cytometry, a recent established approach based on inductively coupled plasma time-of-flight mass spectrometry and a single-cell sample introduction system, allows simultaneous quantification of >50 proteins or protein modifications at single-cell resolution, enabling the profiling of complex cellular behaviors in highly heterogeneous samples. In mass cytometry, metal isotope-tagged antibodies are used to label proteins or protein modifications in cells. During the sample acquisition, each stained single cell is vaporized, atomized, and ionized. The metals in the formed ion cloud are quantitatively analyzed by the mass spectrometer to yield a high-dimensional single-cell proteomic readout. Previous researches have introduced mass cytometry as a versatile approach to assess the signaling network states of over 30 phosphorylation sites in millions of single cells. Relationships between all pairs of measured phosphorylation sites can be computed to infer network responses to a stimulus or to trace the network reshaping through a phenotypical transition. In combination with high-throughput screening assays, these types of experiments have revealed novel signaling mechanisms involved in cancer progression and drug resistance.

However, single-cell signaling network analysis has not been transformed to study patient-derived tumor samples in clinical cancer research. Similar to many other type of single cell approaches, mass cytometry faces a sensitivity limitation that ~300 metal tagged antibody targeting the same epitope has to present in one cell to generate a detectable signal. For single-cell signaling network profiling in cancer samples, the sensitivity limitation has an even higher impact as the basal levels of phosphorylation sites (i.e., without additional stimulation) typically do not reach the detection limit for mass cytometry, especially in cells of smaller volume, such as infiltrated T cells in a tumor sample. In addition, phosphorylated residues are difficult to be fully preserved in tissue samples. Factors such as temperature fluctuation and fixation protocols may further reduce the phosphorylation levels before samples can be analyzed. In the imaging mode of mass cytometry analysis (IMC), the imaging resolution has to be compromised in compensation of low sensitivity that the spatial cues determining a signaling outcome is difficult to be identified.

DNA nanodevice has been recently invented that undergoes repeated in situ concatenation in thermocycling conditions. Combining this device with a newly developed photo-crosslinking strategy based on 3-cyanovinylcarbazole phosphoramidite (CNvK) modification, the method has been successfully implemented to amplify the mass cytometry signal to address its sensitivity bottleneck. This has allowed comprehending cell state and predicting cell fate in biological or clinical samples.

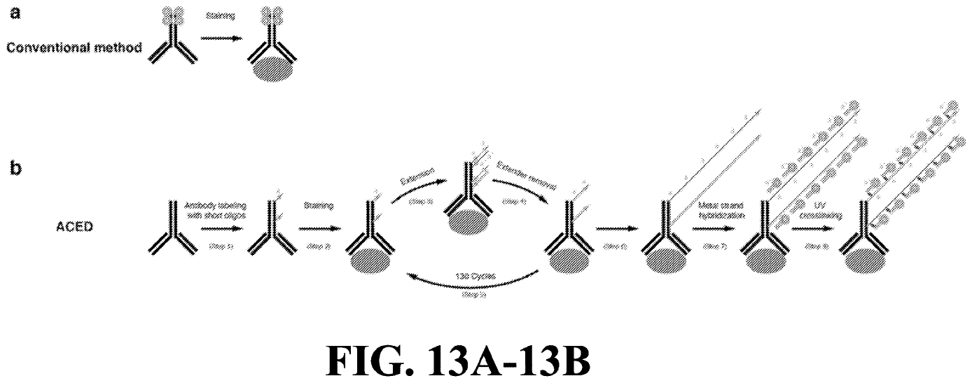

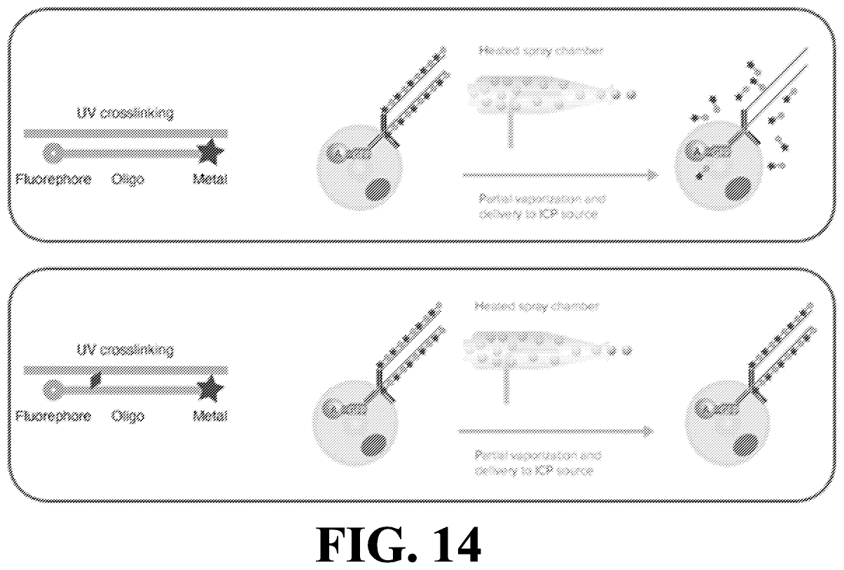

Method. In the conventional mass cytometry method, metal isotopes are first chelated into a maleimide-modified diethylenetriamine pentaacetate (DTPA) polymer that is subsequently conjugated to the reactive cysteine residues located on the hinge region of a partially reduced antibody1. With this approach, only a few modification sites presenting on each antibody can be conjugated, carrying limited number of metal ions (FIG. 13A). To increase the sensitivity of mass cytometric analysis, the main innovation of the novel approach, termed amplification by cyclic extension of DNA oligo (ACED), is to engineer DNA nano-devices that create unlimited repeats of metal probe hybridization sites in situ (FIG. 13B). In ACED, antibodies targeting the protein of interest were conjugated with short DNA oligo initiators (TT-a, 11-mer) that maintains antibody binding affinity, maximizes antibody diffusion capability intracellularly, and reduces nonspecific bindings that are often seen in methods applying long oligo (>40-mer) conjugates14,15 (FIG. 13B, step 1). Conjugated antibodies are mixed in a staining solution and applied on cell suspensions for cell surface or intracellular marker staining (step 2). Next, an extender with two complementary repeats of the initiator sequence (a′-T-a′, 19-mer) is introduced to the stained cells. At low temperature, the extender and initiator hybridize to allow BST polymerase-medicated strand extension (forming TT-a-A-a, step 3). By increasing the reaction temperature, extenders are removed to expose the single-stranded extended probe (step 4). The thermal cycles are then repeated in a desired number of rounds to successively elongate the probe conjugated to the antibody (step 5) that creates hundreds of a-A repeats on each antibody modification site (step 6). DTPA polymers with chelated Ln3+ metal ions are conjugated to detection strands with the sequence of a′-T-a′ through maleimide-thiol reaction that can subsequently hybridize to the extended DNA probes on an antibody (step 7), each occupying one of metal probe binding site (a-A-a). A short-time (1 second) ultraviolet (UV) light exposure activates the 3-cyanovinylcarbazole phosphoramidite (CNVK) photo-cross-linker on the metal probe strand that creates covalent binding between the hybridized DNA molecules and allows DTPA polymers to be attached to the antibody (step 8) (FIG. 14).

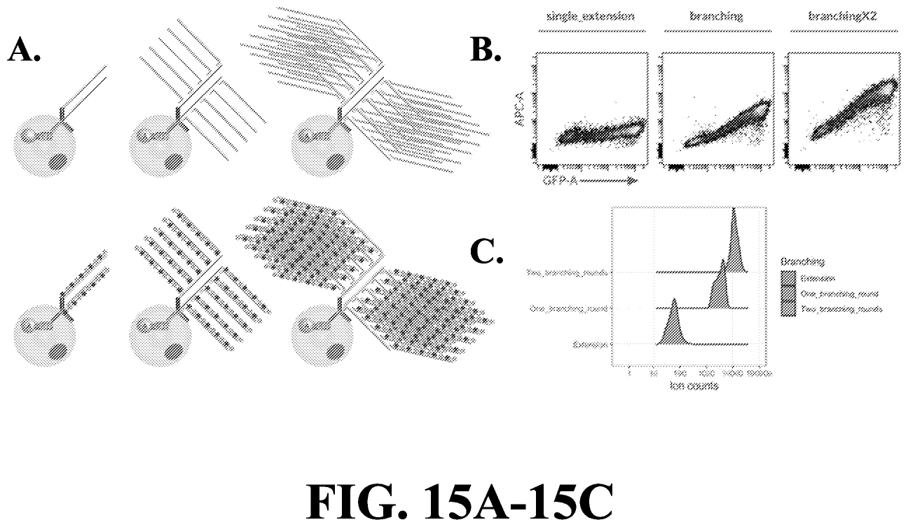

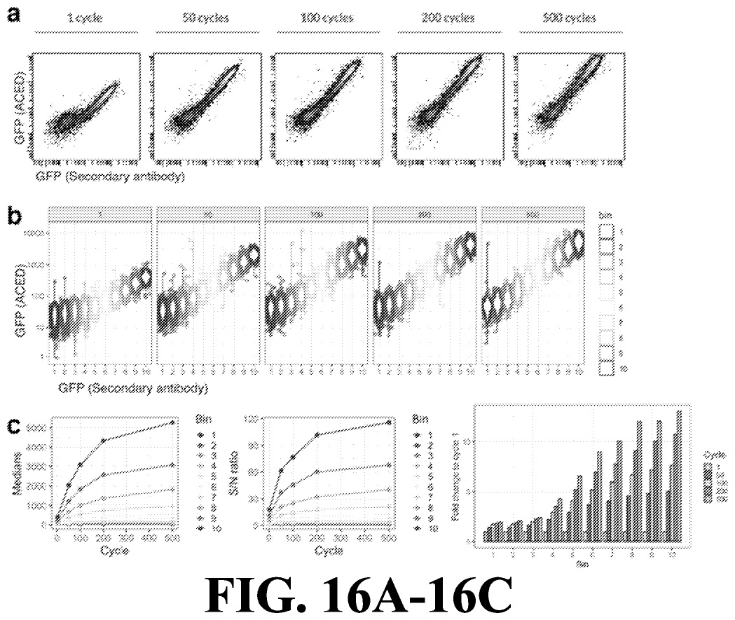

To validate the specificity and to quantify the amplification power of ACED on mass cytometric analysis, this system was applied to the human embryonic kidney HEK293T cells that transiently overexpress green fluorescent protein GFP with a high expression gradient. As expected, ACED-amplified signal of GFP antibody largely correlated with the secondary antibody signal targeting the primary GFP antibody, confirming the specificity of the established DNA device in intracellular antibody signal amplification (FIG. 15A). Through a time-series analysis over 500 thermo cycles, the ACED approach consistently showed amplified mass cytometry signal over the thermocycling time course (FIG. 15A). Quantification using the binned data according to GFP expression level (FIG. 15B), a 13-fold amplification strength and a six-fold signal-to-noise ratio enhancement were observed in samples with 500 rounds amplification, compared to the unamplified sample (FIG. 15C). To detect and quantify ultra-low abundance proteins, linear ACED amplification might be inefficient and time-consuming. For these proteins, a branching amplification strategy was developed that, through iterative rounds of ACED reaction, the amplification power can be exponentially increased. Quantifying the branching results from mass cytometry analysis, a 17-fold amplification for each branching round could be observed (FIG. 16).

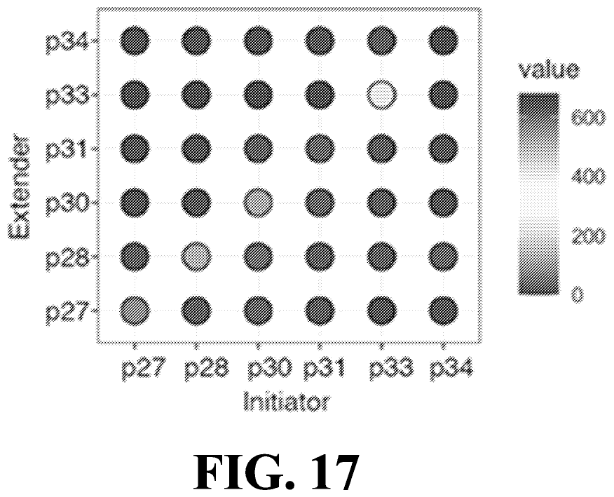

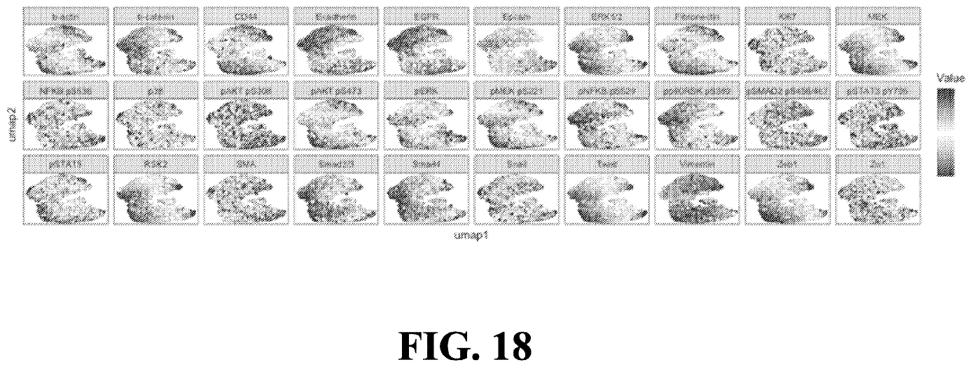

Ultimately, unlimited signal amplification can be achieved with multiple branching reactions. To multiplex the ACED method, high orthogonality between DNA probes targeting different antibodies is required. 50 orthogonal ACED barcode sequences were designed that covers the full capacity of mass cytometry. In a proof-of-concept test, six initiator probes were cross reacted with six extender probes to thoroughly assess their orthogonality (FIG. 17). Only the matching pairs of initiator and extender sequence generated detectable signal in the follow-up mass cytometry analysis (FIG. 17). The 30-plex signal amplification was then performed on a mouse Py2T cell line that underwent epithelial-to-mesenchymal transition (EMT) during a 7-day time course. With ACED, key signature molecules including E-cadherin, vimentin, Smad2/3, Smad4, and most importantly, the transcriptional factors Snail/Slug, Zeb1, could be detected, differentiated and quantified during the progression of the EMT process (FIG. 18). A dimensional reduction analysis using uniform manifold approximation and projection (UMAP) projected the EMT progression trajectory through the time course that validated ACED as a robust technological platform to precisely amplify mass cytometry signal, particularly for low abundance proteins (FIG. 18).

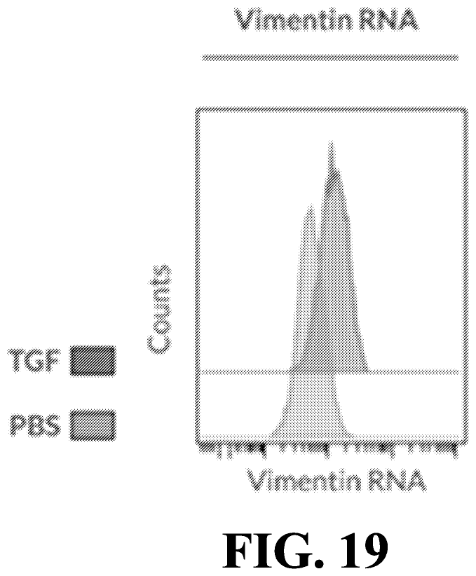

Beside protein detection, in the most recently analysis, it was demonstrated that the method can be applied to differentiate the RNA abundances at single-cell resolution. An average of 2.5-fold increase was detected of vimentin mRNA levels in Py2T cells treated with TGF-β, compared to the untreated (PBS) cells (FIG. 19).

### Example 4: Scenario 3: TCE Combined With Flow Cytometry and Flow Sorting

Flow cytometry and flow sorting techniques involve suspending a cell sample in the fluid of the flow cytometer instrument in order to detect and measure the physical and chemical characteristics of the sample. Despite recent developments in flow cytometry and flow sorting, the method still suffered from low sensitivity and low multiplexity for detection and sorting.

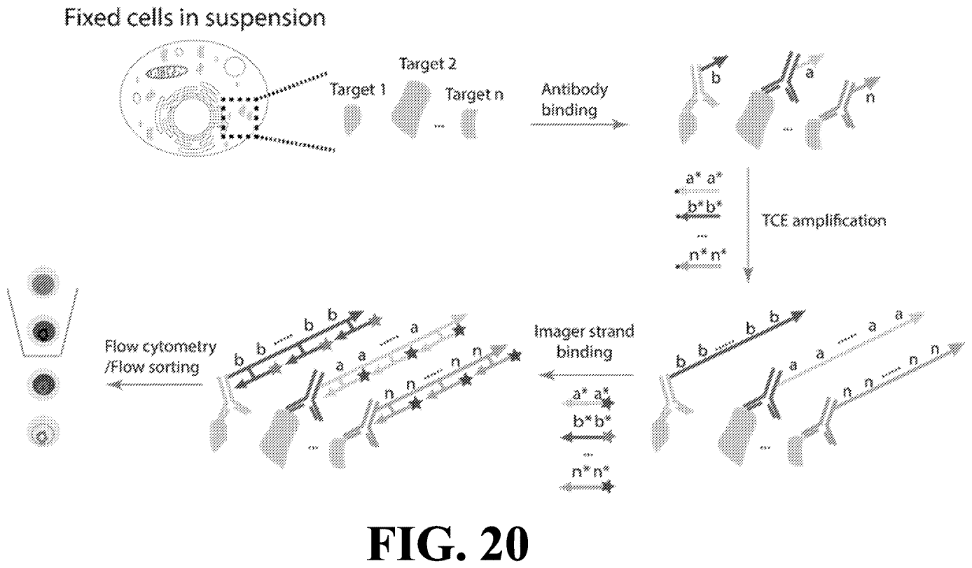

TCE was applied to amplify the signal and improve multiplexity for flow related techniques (FIG. 20). Specifically, the single suspended cells were fixed using fixatives. Antibodies targeting proteins of interests were then added to the sample. The antibodies were conjugated with different orthogonal sequences. TCE amplification was performed to extend the conjugated DNA in situ. Imagers with different fluorophores that can bind to the different concatemers was then added to the samples. Flow cytometry was used to measure the intensity of different fluorophores, revealing their target abundance.

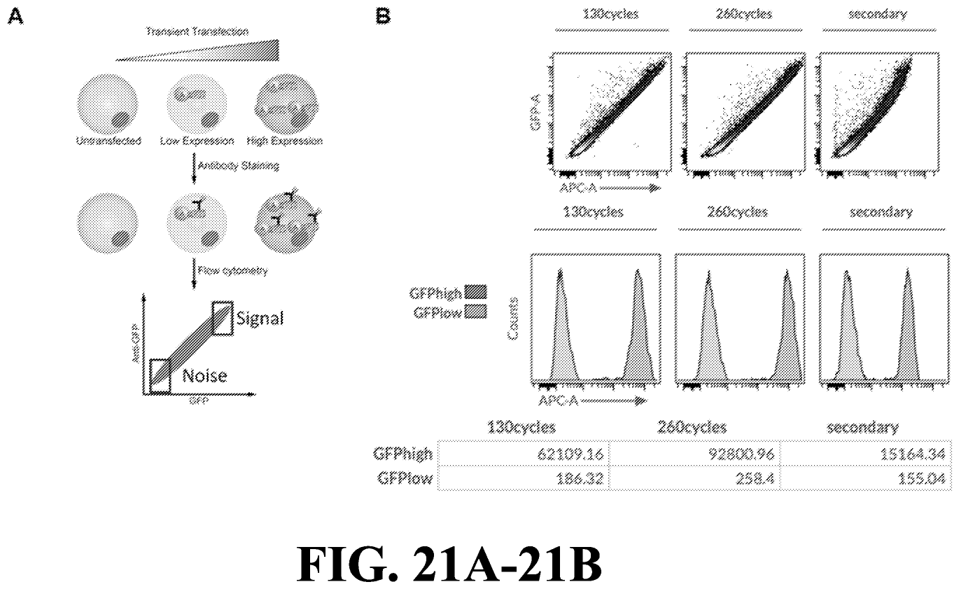

As a proof of concept, HEK293T cells was transfected with GFP mRNA and performed immunostaining with anti-GFP antibodies (FIG. 21A). Signals were then either amplified with 130 cycles or 260 cycles of TCE or amplified with secondary antibodies. Flow cytometry measurement was performed of the single cell suspension samples (FIG. 21B). The absolute signal intensity was compared and also the signal intensity correlation with the GFP fluorescent signal. The results indicate that TCE flow has higher signal amplification and signal to noise ratio compared with the secondary antibody which is considered as the gold standard.

### Example 5: Scenario 4: TCE Combined With Immunoblotting

The immunoblotting techniques use gel electrophoresis to separate proteins from a sample, which are then transferred to a membrane. Antibodies specifically directed against the target protein bind to the membrane and are detected with a chemical or radioactive tag. It is used for detecting specific proteins in samples. One of the key constraint of immunoblotting is the lack of multiplexity as the host species of secondary antibody is limited.

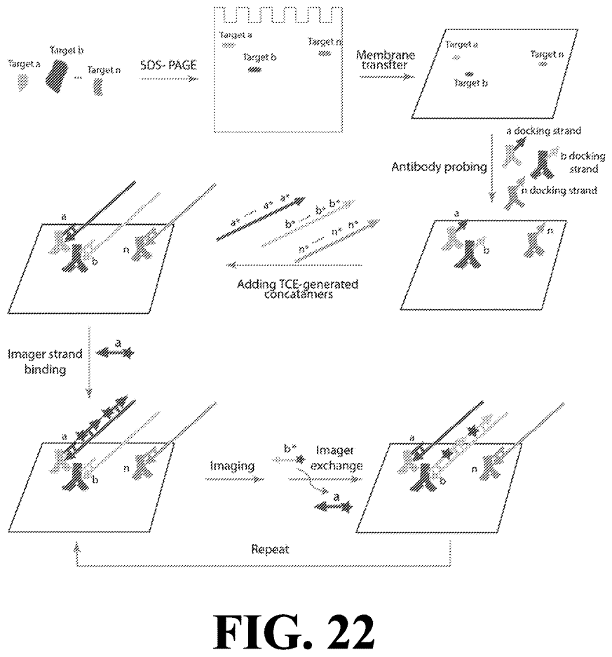

To overcome this limitation, TCE was applied to immunoblotting to allow for much higher multiplexity (FIG. 22). Specifically, orthogonal docking strands were conjugated onto the primary antibodies. After the targets were transferred to membrane post gel electrophoresis, we added the antibodies to the membrane which allows the antibodies to bind to their targets. TCE was used to generate long concatemers in test tube which can bind to the different docking strand respectively. The concatemers were then added to the membrane. After concatemer binding, fluorophore conjugated imagers were added targeting different concatemers to the membrane which allowed for visualize the target by fluorescence imaging. Imager exchange was performed to quickly visualize other targets on the same membrane.

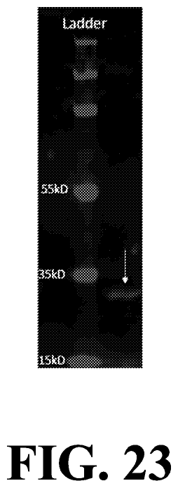

As a proof-of-concept demonstration, GFP protein immunoblotting was performed with ex situ generated concatemers and fluorophore conjugated imagers (FIG. 23). The GFP target band was visualized on the membrane with no nonspecific band.

All patents and other publications identified are expressly incorporated herein by reference for the purpose of describing and disclosing, for example, the methodologies described in such publications that might be used in connection with the present invention. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents is based on the information available to the applicants and does not constitute any admission as to the correctness of the dates or contents of these documents.

What is claimed is:

1. A method of labeling a target-binding ligand, the method comprising:
(a) providing a conjugate of the target-binding ligand and a first oligonucleotide primer;
(b) in a reaction mixture, contacting the conjugated first oligonucleotide primer with a first single-stranded extender template and a nucleic acid polymerase enzyme under conditions permitting hybridization and extension of the first oligonucleotide primer using the first single-stranded extender template;
(c) heating the reaction mixture of step (b) to separate the first single-stranded extender template from extended first oligonucleotide primer produced in step (b);
(d) repeating steps (b) and (c) at least once, thereby generating a concatemer comprising repeats of oligonucleotide primer sequence, conjugated to the target-binding ligand.

2. The method of claim 1, wherein the first single-stranded extender template comprises a concatemer of at least two head-to-tail copies of a sequence complementary to the first oligonucleotide primer, and a 3′ chain terminator, such that the extender template is not extended by the polymerase.

3. The method of claim 1 or claim 2, further comprising, after at least one repeat of steps (b)-(d), the steps of:
(i) adding a second oligonucleotide primer and a second single-stranded extender template oligonucleotide, wherein the second oligonucleotide primer comprises, in 5′ to 3′ order, sequence complementary to the first oligonucleotide primer, and sequence complementary to the second single-stranded extender template, wherein the second single-stranded extender template comprises a 3′ chain terminator;
(ii) heating the reaction mixture to separate nucleic acid strands and cooling to permit hybridization of the second oligonucleotide primer to concatemer generated in steps (b)-(d) and hybridization of the second single-stranded extender template to the second oligonucleotide primer;
(iii) extending the second oligonucleotide primer using the second single-stranded extended template oligonucleotide as template; and
(iv) repeating steps (ii) and (iii) at least once, thereby generating a concatemer comprising repeats of the second oligonucleotide primer.

4. The method of claim 1 or claim 2, further comprising, after step (d):
(i) adding, under conditions permitting hybridization, a nucleic acid comprising: (1) a second concatemer comprised of repeats of a second oligonucleotide sequence; and (2), a sequence complementary to the first oligonucleotide primer, such that a plurality of molecules of the nucleic acid comprising the second concatemer hybridizes via the sequence complementary to the first oligonucleotide primer to the concatemer formed in step (d).

5. The method of any one of claims 1-4, wherein the method is performed in situ on a cell or tissue sample comprising or being assayed for target ligand.

6. The method of claim 5, wherein the cell or tissue sample is fixed.

7. The method of claim 5 or claim 6, wherein the tissue sample is paraffin embedded.

8. The method of any one of claims 1-7, further comprising contacting a concatemer generated in prior steps with a labeled nucleic acid probe.

9. The method of claim 8, wherein the nucleic acid probe comprises sequence complementary to a concatemer repeat.

10. The method of claim 8 or claim 9, wherein the nucleic acid probe comprises sequence complementary to the first oligonucleotide primer.

11. The method of any one of claims 3, or 5-7, further comprising contacting a concatemer comprised of repeats of the second oligonucleotide primer with a labeled nucleic acid probe, wherein the nucleic acid probe comprises sequence complementary to the second oligonucleotide primer.

12. The method of any one of claims 4, or 5-7, further comprising contacting a concatemer comprised of repeats of the second oligonucleotide sequence with a labeled nucleic acid probe, wherein the nucleic acid probe comprises sequence complementary to the second oligonucleotide sequence.

13. The method of any one of claims 8-12, wherein a plurality of labeled nucleic acid probe molecules hybridizes to a plurality of concatemeric repeats.

14. The method of any one of claims 1-8, wherein the target-binding ligand comprises an antibody or antigen-binding fragment thereof.

15. The method of any one of claims 1-14, wherein a plurality of different target-binding ligands are labeled in multiplex via different orthogonal oligonucleotide primer/single-stranded extender template pairs.

16. A method of detecting a target molecule in situ in a preparation of cells or tissue, the method comprising:
(a) contacting the preparation of cells or tissue with a target-binding ligand conjugated to a first oligonucleotide primer, under conditions permitting specific binding of the target-binding ligand to the target molecule;
(b) adding to the preparation of cells or tissue a reaction mixture comprising a first single-stranded extender template and a nucleic acid polymerase enzyme under conditions permitting hybridization and extension of the first oligonucleotide primer using the first single-stranded extender template;
(c) heating the reaction mixture of step (b) to separate the first single-stranded extender template from extended first oligonucleotide primer produced in step (b);
(d) repeating steps (b) and (c) at least once, thereby generating a concatemer comprising repeats of the first oligonucleotide primer sequence, conjugated to the target-binding ligand;
(e) contacting the concatemer generated in step (d) with a labeled nucleic acid probe; and
(f) detecting labeled nucleic acid probe, wherein detection of labeled nucleic acid probe indicates the presence and location of the target molecule in the preparation of cells or tissue.

17. The method of claim 16, wherein the first single-stranded extender template comprises a concatemer of at least two head-to-tail copies of a sequence complementary to the first oligonucleotide primer, and a 3′ chain terminator, such that the extender template is not extended by the polymerase.

18. The method of claim 16 or claim 17, wherein the target-binding ligand comprises an antibody or antigen-binding fragment thereof.

19. The method of any one of claims 16-18, wherein the labeled nucleic acid probe comprises sequence complementary to the first oligonucleotide primer.

20. The method of any one of claims 16-19, wherein the labeled nucleic acid probe comprises sequence complementary to a concatemer repeat.

21. The method of any one of claims 16-20, wherein a plurality of labeled nucleic acid probe molecules hybridizes to a plurality of concatemer repeats conjugated to the target-binding ligand.

22. The method of any one of claims 16-18, further comprising the steps, after step (d), and before step (e), of:
(i) adding a second oligonucleotide primer and a second single-stranded extender template oligonucleotide, wherein the second oligonucleotide primer comprises, in 5′ to 3′ order, sequence complementary to the first oligonucleotide primer, and sequence complementary to the second single-stranded extender template, wherein the second single-stranded extender template comprises a 3′ chain terminator;
(ii) heating the reaction mixture to separate nucleic acid strands and cooling to permit hybridization of the second oligonucleotide primer to concatemer generated in step (d) and hybridization of the second single-stranded extender template to the second oligonucleotide primer;
(iii) extending the second oligonucleotide primer using the second single-stranded extended template oligonucleotide as template; and
(iv) repeating steps (ii) and (iii) at least once, thereby generating a concatemer comprising repeats of the second oligonucleotide primer.

23. The method of claim 22, wherein the labeled nucleic acid probe comprises sequence complementary to the second oligonucleotide primer.

24. The method of claim 22 or 23, wherein a plurality of labeled nucleic acid probe molecules hybridizes to a plurality of concatemer repeats conjugated to the target-binding ligand.

25. The method of any one of claims 16-18, further comprising, after step (d) and before step (e):
(i) adding, under conditions permitting hybridization, a nucleic acid comprising: (1) a second concatemer comprised of repeats of a second oligonucleotide sequence; and (2), a sequence complementary to the first oligonucleotide primer, such that a plurality of molecules of the nucleic acid comprising the second concatemer hybridizes via the sequence complementary to the first oligonucleotide primer to the concatemer comprising repeats of the first oligonucleotide primer sequence formed in step (d).

26. The method of claim 25, wherein the labeled nucleic acid probe comprises sequence complementary to the second oligonucleotide sequence.

27. The method of claim 25 or 26, wherein a plurality of labeled nucleic acid probe molecules hybridizes to a plurality of second concatemer repeats.

28. The method of any one of claims 16-27, wherein the preparation of cells or tissue is fixed.

29. The method of claim 28, wherein the preparation of tissue is paraffin embedded.

30. A method of labeling a set of target-binding ligands, the method comprising:
(a) providing a set of orthogonal first oligonucleotide primer and first single-stranded extender template oligonucleotide pairs, wherein the first oligonucleotide primer of each pair has a different sequence from other first oligonucleotide primers in the set, and is conjugated to a different member of a set of target-binding ligands;
(b) in a reaction mixture, contacting the conjugated first oligonucleotide primers with the first single-stranded extender templates of the set of orthogonal pairs and a nucleic acid polymerase under conditions permitting hybridization and extension of the first oligonucleotide primers using the first single-stranded extender templates of the set of orthogonal pairs;
(c) heating the reaction mixture of step (b) to separate the first single-stranded extender templates from extended first oligonucleotide primers produced in step (b);
(d) repeating steps (b) and (c) at least once, thereby generating, on each member of the set of target ligands, a concatemer comprising repeats of oligonucleotide primer sequence, conjugated to the target-binding ligand.

31. The method of claim 30, wherein the first single-stranded extender template in each orthogonal first oligonucleotide primer/first single-stranded extender pair comprises a concatemer of at least two head-to-tail copies of a sequence complementary to the first oligonucleotide primer, and a 3′ chain terminator, such that the extender template is not extended by the polymerase.

32. The method of claim 30 or claim 31, further comprising, after step (d), the steps of:
(i) adding a second set of orthogonal second oligonucleotide primer, second single-stranded extender template oligonucleotide pairs, wherein the second oligonucleotide primer in each pair comprises, in 5′ to 3′ order, sequence complementary to a member of the set of first oligonucleotide primers, and sequence complementary to the second single-stranded extender template of the second set of orthogonal oligonucleotide pairs, wherein each second single-stranded extender template comprises a 3′ chain terminator;
(ii) heating the reaction mixture to separate nucleic acid strands and cooling to permit hybridization of the second oligonucleotide primers to concatemers generated in steps (b)-(d) and hybridization of the second single-stranded extender templates to the second oligonucleotide primers;
(iii) extending the second oligonucleotide primers using the second single-stranded extended template oligonucleotides as template; and
(iv) repeating steps (ii) and (iii) at least once, thereby generating, on respective members of the set of target-binding ligands, a concatemer comprising repeats of a respective second oligonucleotide primer.

33. The method of claim 30 or 31, further comprising contacting concatemers generated in prior steps with a plurality of labeled nucleic acid probes, wherein each different probe is labeled with a distinguishable detectable label moiety, and wherein members of the plurality of labeled nucleic acid probes comprise sequence complementary to respective members of the set of first oligonucleotide primers.

34. The method of claim 32, further comprising contacting concatemers generated in step (iv) with a plurality of labeled nucleic acid probes, wherein each different probe is labeled with a distinguishable detectable label moiety, and wherein members of the plurality of labeled nucleic acid probes comprise sequence complementary to respective members of the set of second oligonucleotide primers.

35. The method of claim 33 or 34, wherein, for respective members of the set of target-binding ligands, a plurality of labeled nucleic acid probe molecules hybridizes to a plurality of concatemeric repeats.

36. The method of any one of claims 30-35, wherein the method is performed in situ on a cell or tissue sample comprising or being assayed for target ligand.

37. The method of claim 36, wherein the preparation of cells or tissue is fixed.

38. The method of claim 37, wherein the preparation of tissue is paraffin embedded.

39. The method of any one of claims 30-38, wherein the target-binding ligand comprises an antibody or antigen-binding fragment thereof.

40. A method of detecting a set of target molecules in situ in a preparation of cells or tissue, the method comprising:
(a) providing a set of target-binding ligand molecules, wherein members of the set of target-binding ligand molecules are conjugated to respective first oligonucleotide primer members of a set of orthogonal first oligonucleotide primer and first single-stranded extender template oligonucleotide pairs, wherein the first oligonucleotide primer of each pair has a different sequence from other first oligonucleotide primers in the set, and is conjugated to a different member of the set of target-binding ligand molecules;
(b) contacting the preparation of cells or tissue with the set of target-binding ligand molecules under conditions permitting specific binding of the target-binding ligand molecules to target molecules present in the preparation of cells or tissue;
(c) adding to the preparation of cells or tissue a reaction mixture comprising first single-stranded extender templates of the set of orthogonal first oligonucleotide primer and first single-stranded extender template oligonucleotide pairs, and a nucleic acid polymerase, under conditions permitting hybridization and extension of the first oligonucleotide primers using the respective first single-stranded extender templates;
(d) heating the reaction mixture of step (c) to separate the first single-stranded extender templates from extended first oligonucleotide primers produced in step (c);
(e) repeating steps (c) and (d) at least once, thereby generating, on respective members of the set of target-binding ligand molecules, a concatemer comprising repeats of the respective first oligonucleotide primer sequence, conjugated to the target-binding ligand molecule;
(f) contacting the concatemers generated in step (e) with a plurality of distinguishably-labeled nucleic acid probes; and
(g) detecting labeled nucleic acid probes, wherein detection of labeled nucleic acid probes indicates the presence and location of the target molecules in the preparation of cells or tissue.

41. The method of claim 40, wherein the first single-stranded extender template comprises a concatemer of at least two head-to-tail copies of a sequence complementary to the first oligonucleotide primer, and a 3′ chain terminator, such that the extender template is not extended by the polymerase.

42. The method of claim 40 or claim 41, further comprising, after step (e), and before step (f), the steps of:
(i) adding a second set of orthogonal second oligonucleotide primer, second single-stranded extender template oligonucleotide pairs, wherein the second oligonucleotide primer in each pair comprises, in 5′ to 3′ order, sequence complementary to a member of the set of first oligonucleotide primers, and sequence complementary to the second single-stranded extender template of the second set of orthogonal oligonucleotide pairs, wherein each second single-stranded extender template comprises a 3′ chain terminator;
(ii) heating the reaction mixture to separate nucleic acid strands and cooling to permit hybridization of the second oligonucleotide primers to concatemers generated in steps (c)-(e) and hybridization of the second single-stranded extender templates to the second oligonucleotide primers;
(iii) extending the second oligonucleotide primers using the second single-stranded extended template oligonucleotides as template; and
(iv) repeating steps (ii) and (iii) at least once, thereby generating, on respective members of the set of target-binding ligands, a concatemer comprising repeats of a respective second oligonucleotide primer.

43. The method of any one of claims 40-42, wherein the plurality of distinguishably-labeled nucleic acid probes comprises sequences complementary to the respective first oligonucleotide primer members in the set of orthogonal first oligonucleotide primer and first single-stranded extender template oligonucleotide pairs.

44. The method of claim 42, wherein the plurality of distinguishably-labeled nucleic acid probes comprises sequences complementary to respective second oligonucleotide primer members of the second set of orthogonal second oligonucleotide primer, second single-stranded extender template oligonucleotide pairs.

45. The method of any one of claims 40-44, wherein the target-binding ligand comprises an antibody or antigen-binding fragment thereof.

46. The method of any one of claims 40-45, wherein a plurality of labeled nucleic acid probe molecules hybridizes to a plurality of concatemer repeats conjugated to the target-binding ligand.

47. A kit comprising reagents for performing the methods of any one of claims 1-46.

48. A target-binding ligand comprising a concatemer produced by the method of any one of claims 1-15.

49. The target-binding ligand of claim 48, wherein the target-binding ligand comprises an antibody or antigen-binding fragment thereof.

50. A method of generating a nucleic acid strand comprising concatemeric repeats of a given sequence, the method comprising:
a) providing in a reaction mixture, a first oligonucleotide primer and a first extender template oligonucleotide comprising a concatemer of at least two head-to-tail copies of sequence complementary to the first oligonucleotide primer, wherein the first extender template comprises a chain terminator at its 3′ end;
b) incubating the reaction mixture under conditions permitting hybridization of the first oligonucleotide primer to the first extender template oligonucleotide;
c) extending the hybridized first primer with a nucleic acid polymerase enzyme, thereby generating an extended nucleic acid strand complementary to the first extender template;
d) heating the reaction mixture to separate the extended nucleic acid strand complementary to the first extender template from the first extender template;
e) cooling the heated products to permit hybridization of the first extender template oligonucleotide to the extended nucleic acid strand complementary to the first extender template generated in step (c);
f) extending the extended nucleic acid strand complementary to the first extender template generated in step (c) with the polymerase, thereby generating an extended nucleic acid strand comprising concatemeric repeats of the sequence comprised by the first oligonucleotide primer.

51. The method of claim 50, further comprising repeating steps (d) to (f) at least once.

52. The method of claim 51, comprising repeating steps (d) to (f) at least n times, wherein each iteration of steps (d) to (f) increases the concatemeric nucleic acid length by one concatemeric repeat.

53. The method of any one of claims 50-52, wherein step (a) comprises providing a set of orthogonal first oligonucleotide primer and first extender template oligonucleotide pairs, such that steps (b)-(f) generate a multiplex set of concatemers, each comprising concatemeric repeats of the respective orthogonal oligonucleotide primer sequences.

54. The method of any one of claims 51-53, further comprising, after one or more repetitions of steps (d) to (f):
i) adding a second oligonucleotide primer and a second extender template oligonucleotide to the reaction, wherein the second oligonucleotide primer comprises a 5′ proximal sequence complementary to the first oligonucleotide primer sequence and a 3′ proximal sequence complementary to one of at least two repeat portions of the second extender template oligonucleotide, wherein the second extender template oligonucleotide comprises at least two head-to-tail repeats of sequence complementary to the second oligonucleotide primer and a chain terminator at its 3′ end;
ii) hybridizing the second oligonucleotide primer to one or more concatemeric repeats on a concatemer formed after one or more repetitions of steps (d) to (f);
iii) hybridizing the second extender template oligonucleotide to the second oligonucleotide primer;
iv) extending the second oligonucleotide primer with the polymerase enzyme, thereby generating an extended nucleic acid strand complementary to the second extender template;
v) heating the reaction mixture to separate the extended nucleic acid strand complementary to the second extender template from the second extender template;
vi) cooling the heated products to permit hybridization of the second extender template oligonucleotide to the extended nucleic acid strand complementary to the second extender template generated in step (iv);
vii) extending the extended nucleic acid strand complementary to the second extender template generated in step (iv) with the polymerase, thereby generating an extended nucleic acid strand comprising concatemeric repeats of the sequence comprised by the second oligonucleotide primer.

55. The method of claim 54, further comprising repeating steps (v) to (vii) at least once.

56. The method of claim 54 or 55, wherein step (i) comprises providing a set of orthogonal second oligonucleotide primer and second extender template oligonucleotide pairs, such that steps (ii)-(vii) generate a multiplex set of concatemers, each comprising concatemeric repeats of the respective orthogonal oligonucleotide primer sequences.

57. The method of any one of claims 50-56, wherein the first oligonucleotide primer is conjugated to a target-binding ligand via its 5′ end.

58. The method of claim 57, wherein the target-binding ligand is a polypeptide.

59. The method of claim 58, wherein the polypeptide comprises an antibody or antigen-binding fragment thereof.

60. The method of any one of claims 57-59, further comprising contacting the target-binding ligand with a cell or tissue preparation comprising or being assayed for the presence of the target.

61. The method of claim 60, wherein the contacting is performed before concatemer-generating steps (a)-(f) or (i)-(vii).

62. The method of any one of claims 50-61, further comprising contacting an extended nucleic acid strand comprising concatemeric repeats generated according to steps (a)-(f) and/or (i)-(vii) with a nucleic acid probe comprising the complement of a concatemeric repeat sequence, and a detectable label.

63. The method of any one of claims 50-53, wherein the method generates linear concatemers comprising the first oligonucleotide primer sequence.

64. The method of claim 54, wherein the method generates branched concatemers comprising concatemers of the first oligonucleotide primer sequence, complexed with concatemers of the second oligonucleotide primer sequence.

65. The method of any one of claims 50-64, wherein the method is performed in contact with a cell or tissue preparation.

66. The method of claim 65, wherein the cell or tissue preparation is fixed.

67. A method of labeling a target-binding ligand, the method comprising:
performing the method of any one of claims 50-65, wherein the first oligonucleotide primer is conjugated to the target-binding ligand.

68. The method of claim 67, wherein the target-binding ligand comprises an antibody or antigen-binding fragment thereof.

69. The method of claim 67 or 68, wherein the first oligonucleotide primer is conjugated to the target binding ligand in a manner that permits extension of the primer from its 3′ end.

70. The method of any one of claims 67-69, wherein the method is performed in contact with a cell or tissue preparation.

71. The method of claim 65, wherein the cell or tissue preparation is fixed.

72. The method of any one of claims 67-71, further comprising contacting the concatemer generated in claim 50 or 51 with a labeled nucleic acid probe, and detecting labeled nucleic acid probe associated with concatemer, wherein detection of labeled nucleic acid probe indicates the presence and location of the target molecule.

73. The method of any one of claims 1-72, wherein the nucleic acid polymerase enzyme is a thermostable nucleic acid polymerase enzyme.

74. A method of generating a nucleic acid molecule, the method comprising:
(a) providing a first oligonucleotide primer;
(b) in a reaction mixture, contacting the first oligonucleotide primer with a first single-stranded extender template and a nucleic acid polymerase enzyme under conditions permitting hybridization and extension of the first oligonucleotide primer using the first single-stranded extender template, wherein the first single-stranded extender template comprises, in a 5′ to 3′ direction, a first extension template element, a sequence complementary to the first oligonucleotide primer and a 3′ chain terminator, such that the extender template is not extended by the polymerase;
(c) heating the reaction mixture of step (b) to separate the first single-stranded extender template from extended first oligonucleotide primer produced in step (b);
(d) contacting the extended first oligonucleotide primer with a second single-stranded extender template under conditions permitting hybridization and extension of the first extended oligonucleotide primer generated in step (b) using the second single-stranded extender template, wherein the second single-stranded extender template comprises, in a 5′ to 3′ direction, a second extension template element, a copy of the first extension template element, and a 3′ chain terminator;
wherein steps (a)-(d) generate a nucleic acid molecule comprising, in 5′ to 3′ order, the first oligonucleotide primer sequence, the complement of the first extension template element, and the complement of the second extension template element.

75. The method of claim 74, wherein the polymerase enzyme is a thermostable polymerase enzyme.

76. The method of claim 74 or claim 75, further including repeating steps (b) and (c) at least once before step (d), wherein each repeat of steps (b) and (c) extends the generated nucleic acid molecule by one copy of the complement of the first extension template element.

77. The method of any one of claims 74-76, further including, at least one iteration of
(e) heating the reaction mixture of step (d) to separate the second single-stranded extender template from extended first oligonucleotide primer produced in step (d); and
(f) repeating step (d),
wherein each iteration of steps (e) and (f) extends the generated nucleic acid molecule by one copy of the complement of the second extension element.

78. The method of any one of claims 74-77, further comprising heating the reaction mixture after step (d) and contacting the nucleic acid molecule generated in steps (a) to (d) with at least one additional single-stranded extender template under conditions permitting hybridization and extension of the generated nucleic acid molecule, wherein the additional single-stranded extender template comprises, in a 5′ to 3′ direction, an additional extension template element, a copy of the extension template element from the previous round of extension, and a 3′ chain terminator, wherein the generated nucleic acid molecule is extended to include the complement of the additional extension template element.

79. The method of any one of claims 74-78, wherein the first oligonucleotide is conjugated to a target-binding ligand.

80. The method of any one of claims 74-79 which is performed in contact with a cell or tissue sample.

81. The method of any one of claims 74-80, further comprising contacting the generated nucleic acid molecule with a labeled nucleic acid probe comprising sequence of one or more of the extension template elements.

82. A kit for performing one or more of the methods as described herein, the kit comprising:
a) a first oligonucleotide primer or a set of first oligonucleotide primers;
b) a single-stranded extender template or a set of single-stranded extender templates; and
c) an order extension or nucleic acid molecule.

83. The kit of claim 82, wherein the first oligonucleotide primer or the set of first oligonucleotide primers and single stranded extender template or the set of single stranded extender templates are orthogonal sets.

84. The kit of claim 83, wherein the orthogonal sets are optimized to avoid primer/extender cross-talk, primer-dimer formation, and off-target hybridization.

85. The kit of claim 82, wherein the single-stranded extender template or the set of single-stranded extender templates are suitable for performing a concatemer-generated method.

86. The kit of claim 82, further comprising packaging materials for the various components and instructions for use.

87. The kit of claim 82, further comprising one or more target-binding ligand molecules or reagents for conjugating a first oligonucleotide to a target-binding ligand.

88. The kit of claim 82, further comprising a thermostable polymerase, nucleotides, reaction buffer components, and reagents for labeling a nucleic acid probe molecule.

89. The kit of claim 88, further comprising a probe molecule complementary to a concatemeric repeat element.

90. The kit of claim 88, wherein the probe molecule can be complementary to one or more elements in an ordered extension product generated.
